(19) United States
(12) Patent Application Publication
(10) Pub. No.: US 2022/0160750 A1
(43) Pub. Date: May 26, 2022

(54) SINGLE-NUCLEI CHARACTERIZATION OF AMYOTROPHIC LATERAL SCLEROSIS FRONTAL CORTEX

(71) Applicant: President and Fellows of Harvard College, Cambridge, MA (US)

(72) Inventors: Francesco Limone, Cambridge, MA (US); Kevin C. Eggan, Cambridge, MA (US)

(21) Appl. No.: 17/535,070

(22) Filed: Nov. 24, 2021

Related U.S. Application Data
(60) Provisional application No. 63/117,992, filed on Nov. 24, 2020.
(60) Provisional application No. 63/117,997, filed on Nov. 24, 2020.

(51) Int. Cl. A61K 31/713; A61P 25/28

(52) U.S. Cl. CPC A61K 31/713; A61P 25/28

(57) ABSTRACT

Disclosed herein are methods and compositions for treating amyotrophic lateral sclerosis.

### RELATED APPLICATIONS

This application claims the benefit of U.S. Provisional Application Ser. No. 63/117,992 filed on Nov. 24, 2020, and U.S. Provisional Application Ser. No. 63/117,997 filed on Nov. 24, 2020, the entire teachings of which are incorporated herein by reference.

### BACKGROUND OF THE INVENTION

Amyotrophic lateral sclerosis is a rapidly progressive, fatal neuromuscular disease with survival typically limited to 2-5 years from the onset of diagnosis (Taylor J P et al. Nature 2016). Although genetics studies of familial ALS have tremendously increased the understanding of this disease, the vast majority of ALS cases are sporadic, occurring without a family history of disease and most often without a known genetic cause (Wainger B J, Lagier-Tourenne C., Cell Stem Cell 2018; Brown NEJM 2017). Variants in several genes associated with ALS can also contribute to a related neurological disease called Frontotemporal Dementia (FTD), supporting the notion that ALS and FTD represent different clinical manifestations of a shared underlying disease process. In addition to the loss of neurons in the frontal cortex and spinal cord, ALS is characterized by reactive changes in astrocytes and microglia. Notably, non-cell autonomous effects of glial cells, including microglia and oligodendroglia, are key mediators of disease progression in many ALS models.

Bulk RNA-sequencing and unbiased analysis of ALS post-mortem brains has provided several disease associated pathways and tissue-resolution transcriptional signatures that demonstrated clear differences between sporadic ALS and cases of ALS associated with hexanucleotide repeat expansions in C9ORF72 (Prudencio et al. Nature Neuroscience; D'Erchia et al). For instance, C9ORF72-ALS had robust upregulation of transcripts encoding protein chaperones that were validated in a cohort of over 50 C9ORF72-ALS/FTD cases, whereas sporadic ALS had downregulation of transcripts associated with mitochondrial function. However, which cellular subtypes were contributing to these transcriptional changes was not determined and whether there are more subtle changes in cellular states, particularly in less common cell types that were below the limits of detection, remains unresolved. Methods to study cellular heterogeneity at a single-cell level, including Drop-seq, have rapidly advanced and have recently been adapted to profile single-nuclei extracted from frozen tissue samples. Their application to mouse and human post-mortem brain tissue are beginning to emerge, especially for Alzheimer's disease (AD). For instance, single-cell RNA-seq has identified a novel activation response in microglia termed disease-associated microglia (DAM) associated with amyloid plaques. Microglia isolated from a SOD1-G93A mouse model of familial ALS exhibited similar changes (Keren-Shaul et al. Cell 2017). However, a comprehensive view of the complex changes across neuronal and non-neuronal cell types in sporadic ALS has not been performed.

### SUMMARY OF THE INVENTION

Amyotrophic Lateral Sclerosis (ALS) is a rapidly fatal neurodegenerative disorder associated with highly complex cellular and molecular pathological processes, most of which are still poorly understood, that converge to a common clinical phenotype and outcome. Many studies have revealed disease mechanisms underlying inherited forms of ALS, associated with distinct, highly penetrant genetic variants, and have proposed multiple cell types as a causative and/or contributing to neural degeneration. However, which specific cell type might be affected by any of these mechanisms remains unresolved. In the study described herein, single-nucleus transcriptomic analysis was performed of 79,169 nuclei isolated from the frontal cortex of 8 individuals with sporadic ALS (sALS) or age-matched unaffected controls. The study provided an increased resolution of the complex landscape in sALS and allowed for the transcriptional classification of specific disease-related molecular alterations in distinct cellular subpopulations. Notably, a robust activation of cellular stress pathways, previously described in the disease models, was specifically identified in excitatory deep-layer cortico-spinal neurons. Neuronal stress is connected to a shift in oligodendrocyte cells from a myelinating to a neuronally supportive transcriptional state. These changes are also accompanied by a novel reactive state of microglial cells. Overall, the findings of strong neuronal vulnerability and potential compensatory, pro-neuronal changes in oligodendrocytes and microglial advances the knowledge of specific cellular responses in sALS and may foster precision medicine-based treatments.

Disclosed herein are methods of treating a neurodegenerative disease or disorder comprising administering to a subject an agent that modulates neuronal regeneration.

In some embodiments, the agent modulates (e.g., increases) uptake of toxic proteins from intercellular environment. In some embodiments, the agent increases expression of SORL1, e.g., in microglia and/or neurons (e.g., motor neurons). In some embodiments, the neurodegenerative disease or disorder is selected from the group consisting of amyotrophic lateral sclerosis (ALS), frontotemporal dementia (FTD), Alzheimer's disease (AD), and multiple sclerosis (MS). In certain embodiments, the neurodegenerative disease or disorder is ALS.

Also disclosed herein are methods of treating a neurodegenerative disease or disorder comprising administering to a subject an agent that modulates proteasome inhibition toxicity of neurons (e.g., motor neurons).

In some embodiments, the agent increases proteasome activity by reducing the inhibitory activity of the PSMD12 subunit. In some embodiments, the agent protects neurons from proteasome inhibition (e.g., from transitioning to a TDP-43 pathology). In some embodiments, the agent promotes neuron survival. In some embodiments, the agent decreases expression of PSMD12, e.g., in neurons. In some embodiments, the neurodegenerative disease or disorder is selected from the group consisting of amyotrophic lateral sclerosis (ALS), frontotemporal dementia (FTD), Alzheimer's disease (AD), and multiple sclerosis (MS). In certain embodiments, the neurodegenerative disease or disorder is ALS.

Disclosed herein are pharmaceutical compositions comprising an agent and a pharmaceutically acceptable carrier, diluent, or excipient. In some aspects, the agent increases expression of SORL1 in microglia and/or neurons. In other aspects, the agent decreases expression of PSMD12 in neurons.

In some embodiments, the composition modulates the uptake of toxic proteins from an intercellular environment. In some embodiments, the composition protects neurons from proteasome inhibition. In some embodiments, the composition further comprises an agent for treating a neurodegenerative disease or disorder.

Also disclosed herein are methods of screening one or more test agents to identify candidate agents for treating a neurodegenerative disease or condition in a subject. In some embodiments, the methods comprise providing a neuronal cell having decreased expression of SORL1; contacting the cell with one or more test agents; determining if the contacted cell has an increased expression level of SORL1; and identifying the test agent as a candidate agent if the contacted cell has an increased expression level of SORL1. In other embodiments, the methods comprise providing a neuronal cell having increased expression of PSMD12; contacting the cell with one or more test agents; determining if the contacted cell has a decreased expression level of PSMD12; and identifying the test agent as a candidate agent if the contacted cell has a decreased expression level of PSMD12.

In some embodiments, the step of determining if the contacted cell has increased expression levels of SORL1 or decreased levels of PSMD12 comprises measuring SORL1 or PSMD12 protein levels in the contacted cell. The SORL1 and PSMD12 protein levels in the contacted cells may be measured using an ELISA assay.

In some embodiments, the neurodegenerative disease or disorder is selected from the group consisting of amyotrophic lateral sclerosis (ALS), frontotemporal dementia (FTD), Alzheimer's disease (AD), and multiple sclerosis (MS). In certain embodiments, the neurodegenerative disease or disorder is ALS.

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); The ELISA guidebook (Methods in molecular biology 149) by Crowther J. R. (2000); Immunology by Werner Luttmann, published by Elsevier, 2006. Definitions of common terms in molecular biology can also be found in Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Cun-ent Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (3 ed.), Cold Spring Harbor Laboratory Press, Cold Spring Harbor, N.Y., USA (2001) and Davis et al., Basic Methods in Molecular Biology, Elsevier Science Publishing, Inc., New York, USA (1995) which are both incorporated by reference herein in their entireties.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1C demonstrate snRNAseq cell-type characterization and distribution across individuals. FIG. 1A provides two-dimensional t-SNE projections of the whole cohort with expression of broad cell type markers. FIG. 1B shows differential expression of additional cell type specific markers with the percentage of cells expressing the given marker in each cluster. FIG. 1C provides the fraction of each cell type identified in the whole cohort split by diagnosis.

FIGS. 2A-2F demonstrate that excitatory neurons from the ALS cortex present increased expression of stress-related pathways. FIG. 2A shows t-SNE projection of excitatory neurons cluster (ALS n=15,227 nuclei, Control n=17,583 nuclei). FIG. 2B shows t-SNE projection of subclusters identified in excitatory neurons as representing different, biologically relevant neuronal layers. FIG. 2C provides a diagram comparing cortical layer specific DGE between ALS and a control. FIG. 2D provides a Dotplot representing the z-scores for DGEs identified as upregulated in each subgroup of excitatory neurons and showing a strong ALS-related signature in lower cortical layers where Cortico-Spinal Motor Neurons reside. FIG. 2E provides a comparison of genes upregulated in subgroups of excitatory neurons. FIG. 2F provides a Gene Ontology analysis of terms associated with genes upregulated in excitatory neurons of ALS patients with highlighted terms being involved in pathways related to cellular stresses found in ALS patients (analysis performed with gProfiler).

FIGS. 3A-3K demonstrate oligodendroglial cells decrease their myelinating machinery in favor of a neuro-supportive state. FIG. 3A shows t-SNE projection of broad markers of Oligodendrocyte Progenitor Cells (OPCs) and of mature oligodendrocytes. FIG. 3B shows t-SNE projection of oligodendrocyte cluster (ALS n=8,372 nuclei, Control n=11,168 nuclei). FIG. 3C shows t-SNE projection of subclusters identified within oligodendroglial cells. FIG. 3D shows distribution of oligodendroglial cells within clusters by diagnosis. FIG. 3E provides a Gene Ontology analysis of terms associated with genes characteristic of Control-enriched oliglia0 with highlighted terms being involved in myelination (analysis performed with gProfiler). FIG. 3F provides a Gene Ontology analysis of terms associated with genes characteristic of ALS-enriched oliglial with highlighted terms being involved in neuro-supportive functions (analysis performed with gProfiler). FIG. 3G provides Violin plots of representative genes involved in neurosupportive functions upregulated (left) in ALS and of genes involved in myelination downregulated (right) in ALS patients. FIG. 3H provides volcano plot of differentially expressed genes (DEGs) upregulated in oligodendroglia. Highlighted genes have been identified in Gene Ontology terms related to myelination (orange) and/or neuro-supportive functions (green). FIGS. 3I-3J provide Violin plots representing z-scores for selected, statistically significant GO terms. FIG. 3K provides a diagram illustrating proposed shift of oligodendrocytes states in ALS.

FIGS. 4A-4C demonstrate that microglia in ALS patients acquire a reactive state. FIG. 4A shows t-SNE projection of a microglia subcluster (ALS n=759 nuclei, Control n=693 nuclei). FIG. 4B provides a volcano plot of differentially expressed genes (DEGs) upregulated in microglia from ALS. Genes were identified in Gene Ontology terms for endocytosis and exocytosis. FIG. 4C provides a Gene Ontology analysis of terms associated with genes upregulated in ALS microglia with highlighted terms playing an important role in microglial biology and/or pathogenesis of the disease (analysis performed with gProfiler).

FIG. 5 provides a diagram comparing a control motor cortex and an ALS motor cortex.

FIGS. 6A-6G demonstrate subcellular susceptibility to ALS-FTD in the human cortex. FIG. 6A provides a schematic diagram of workflow for isolation of nuclei from cortices of ALS patients and age-matched controls followed by single-cell RNA sequencing and assessment of expression of gene modules associated to neurodegenerative diseases. FIGS. 6B-6D provide Violin plots and t-SNE projection for z-scores for expression of genes associated with the ALS-FTD (FIG. 6B), AD (FIG. 6C) and MS (FIG. 6D) in the different cell types identified in the cortex (bars denote median for each side of the Violin plot). FIGS. 6E-6G provide Violin plots and t-SNE projection for z-scores for expression of genes associated with the ALS-FTD (FIG. 6E), AD (FIG. 6F) and MS (FIG. 6G) in the different subtypes of excitatory neurons (bars denote median for each side of the Violin plot).

FIGS. 7A-7E demonstrate excitatory neurons from ALS cortex present increased expression of stress-related pathways. FIG. 7A provides a schematic of Differential Gene Expression Analysis strategy. FIG. 7B provide a Dotplot representing the scores for DEGenes upregulated in each subgroup of excitatory neurons (DGE0-1) and globally upregulated in all excitatory cells (DGEall). FIG. 7C show Gene Ontology analysis of terms for genes upregulated in CUX2-Exc0 group (DGE0), highlighted terms involved in synaptic biology (CC=Cellular Components). FIG. 7D shows Gene Ontology analysis for genes upregulated in THY1-Exc1 group (DGE1), highlighted terms are stress pathways involved in ALS pathology (CC=Cellular Components). FIG. 7E provides Violin plots representing z-score for selected, statistically significant GO terms from analysis shown in FIG. 7D, in each subgroup (left) and at the global level (right).

FIGS. 8A-8J demonstrate that in ALS, oligodendroglial cells decrease their myelinating machinery in favor of a neuro-supportive state. FIG. 8A shows t-SNE projection of markers of OPCs and oligodendrocytes. FIG. 8B shows t-SNE projection of oligodendroglial cluster (ALS n=8,372 nuclei, Control n=11,168 nuclei). FIG. 8C shows t-SNE projection of subclusters identified within oligodendroglia. FIG. 8D shows distribution of subclusters by diagnosis. FIG. 8E provides Gene Ontology analysis for genes characteristic of Control-enriched oliglia0, highlighted terms involved in myelination (CC=Cellular Components). FIG. 8F provides Gene Ontology analysis for genes characteristic of ALS-enriched oliglial, highlighted terms involved in neuro-supportive functions (CC=Cellular Components). FIG. 8G provides Violin plots of representative genes for neuro-supportive functions (left) and myelination (right). FIG. 8H provides a volcano plot of differentially expressed genes in oligodendroglia. Highlighted genes identified in GO terms related to myelination (orange) and neuro-supportive functions (green). FIG. 8I provides Violin plots representing z-score for selected GO terms and related t-SNE projection. FIG. 8J provides a diagram illustrating a proposed shift of oligodendrocytes states.

FIGS. 9A-9H demonstrate disease-associated microglia signature in ALS. FIG. 9A shows a t-SNE projection of microglia (ALS n=759 nuclei, Control n=693 nuclei). FIGS. 9B-9C provide volcano plots of genes upregulated in microglia from ALS. Genes identified in Gene Ontology terms for endocytosis and exocytosis highlighted in FIG. 9B, genes associated to neurodegenerative diseases highlighted in FIG. 9C (ALS, PD—Parkinson's disease, MS, AD). FIG. 9D provides Violin plots of representative genes upregulated in ALS patients associated with reactive microglia (geometric boxplots represent median and interquantile ranges). FIG. 9E provides a Dotplot representing expression of genes associated with ALS-FTD pathogenesis upregulated in microglia from patients. FIG. 9F provides a Gene Ontology analysis for genes upregulated in ALS microglia, highlighted terms involved in myeloid cells biology and/or pathogenesis of ALS. FIG. 9G provides Violin plots representing z-score for selected, statistically significant GO terms from FIG. 9F. FIG. 9H shows a comparison of genes upregulated in microglia from ALS patients with genes upregulated in microglia in other neurodegenerative diseases.

FIG. 10 provides a graphical abstract and working model. The study highlights cell type specific changes in prefrontal cortex of sporadic ALS patients. Specifically, upregulation of synaptic molecules was identified in excitatory neurons of upper cortical layers, interestingly correlating to hyperexcitability phenotypes seen in patients. Moreover, excitatory neurons of the deeper layers of the cortex, that project to the spinal cord and are most affected by the disease, show higher levels of cellular stresses than other neuronal types. Correspondently, oligodendrocytes transition from a highly myelinating state to a more neuronally engaged state, probably to counteract stressed phenotypes seen in excitatory neurons. At the same time, microglia show a reactive state with specific upregulation of endo-lysosomal pathways.

FIGS. 11A-11H demonstrate technical parameters of snRNAseq and cell-type distribution across individuals. FIG. 11A provides a schematic diagram of cohort of sample and workflow for isolation of nuclei from cortices of ALS patients and age-matched controls followed single-cell RNA sequencing with DropSeq method, library generation and Quality Controls for analysis with Seurat 3.0.2. FIG. 11B shows frozen tissue from one of the individuals sequenced. FIG. 11C shows quality control parameter post-filtering per individual (FC—Frontal Cortex): number of total nuclei detected (barcodes), average number of genes detected per nucleus (nFeatures), and average number of UMIs (Unique Molecular Identifiers) per nucleus (nCounts). FIG. 11D provides two-dimensional t-SNE projections of the whole cohort with expression of broad cell type markers FIG. 11E provides Violin plots of selected cell type specific markers showing normalized gene expression (nUMIs). FIG. 11F shows differential expression of additional cell type specific markers with percentage of cells expressing the given gene in each cluster. FIG. 11G provides two-dimensional t-SNE distribution of whole cohort with identified cell types annotations split by diagnosis (ALS patients n=5, age-matched Controls n=3, n=79,169 total nuclei). FIG. 11H shows fraction of each cell types identified in whole cohort split by diagnosis.

FIGS. 12A-12G demonstrate expression of ALS-FTD associated genes in different cellular subtypes and excitatory neurons subtypes. FIG. 12A provides a Dotplot representing expression of gene associated with the ALS-FTD spectrum in each cell type identified in the whole cortex split by diagnosis. FIG. 12B provides a t-SNE projection of excitatory neurons cluster (ALS n=15,227 nuclei, Control n=17,583 nuclei). FIG. 12C provides a t-SNE projection of subclusters identified in excitatory neurons represents different, biologically relevant neuronal layers (FindNeighbor(res=0.2)). FIG. 12D provides a Dotplot representing percentage of cells expressing broad markers for different cortical layers. FIG. 12E shows distribution of excitatory neurons within subclusters by individual. FIG. 12F provides Get Set Enrichment Analysis for the ALS-FTD associated genes in the lower THY1 excitatory neurons. FIG. 12G provides Get Set Enrichment Analysis for the ALS-FTD associated genes in the upper CUX1 cortical neurons.

FIGS. 13A-13E demonstrate neurons of lower cortical layers express higher levels of stress pathways. FIG. 13A shows a comparison of genes globally upregulated in ALS excitatory neurons (Exc all) with genes upregulated in specific layers: CUX2-exc0, THY1-exc1, FEZF2-exc5 (genes defined as expressed by >10% of cells, 2-FC higher than Control, adjusted p-value<0.05). FIG. 13B provides Gene Ontology analysis of terms associated with genes globally upregulated in excitatory neurons of ALS patients independently of groups (DGEall). FIGS. 13C-13D provide Violin plots representing z-scores for selected, statistically significant GO terms upregulated in lower (FIG. 13C) layers and upper layers (FIG. 13D), in each subgroup (left) and globally (right). FIG. 13E shows a representation of −log 10 (adjusted p-values) of selected GO terms from previous figures for CUX2-Exc0 and THY1-Exc1 groups and globally.

FIG. 14 demonstrates global protein-protein interaction network for genes upregulated in ALS excitatory neurons. Color-coding derived from MLC clustering (4) to identified closely related groups of proteins.

FIGS. 15A-15G demonstrate proteostatic stress in hPSC-derived neurons resembles changes in excitatory neurons from brain of ALS patients. FIG. 15A shows a diagram of neuronal differentiation from Pluripotent Stem Cells and treatment with proteasome inhibitors for bulk RNA-sequencing. FIG. 15B shows quantification of proteasome inhibition. FIG. 15C shows immunofluorescence of TDP-43 localisation after treatment. FIG. 15D provides a Principle Component Analysis plot showing strong effect of treatments compared to untreated controls. FIG. 15E provides a Venn Diagram depicting shared upregulated genes between treated hPSC-derived neurons and excitatory neurons from ALS patients. FIG. 15F shows protein-protein interaction network of shared genes from FIG. 15D. FIG. 15G shows Gene Ontology analysis for shared genes in FIG. 15E, highlighted terms involved in protein folding and neurodegenerative diseases (CC=Cellular Components).

FIGS. 16A-16D show the distribution of oligodendroglial subtypes. FIG. 16A provides a t-SNE projection and corresponding Violin plots of additional broad markers of Oligodendrocyte Progenitor Cells (OPCs) and of mature oligodendrocytes. FIG. 16B provides a t-SNE projection of oligodendroglial cells by individual. FIG. 16C provides a t-SNE projection of subclusters identified within oligodendroglia split by diagnosis (FindNeighbor(res=0.2)). FIG. 16D shows a distribution of oligodendroglia within subclusters by individual.

FIGS. 17A-17G demonstrate oligodendrocytes polarization between myelinating and neurotrophic. FIG. 17A provides a t-SNE projection of broad markers of actively myelinating oligodendrocytes. FIG. 17B provides a t-SNE projection of broad markers of neuro-supportive oligodendrocytes. FIGS. 17C-17D provide Violin plots representing relevant z-score for selected GO terms by cluster. FIG. 17E provides a Dotplot representing genes characteristic of maturation and development of OPCs into highly myelinating oligodendrocytes in each subcluster split by diagnosis. FIG. 17F shows a Gene Ontology analysis of terms associated with genes downregulated in ALS oligodendrocytes, highlighted terms involved in myelination (CC=Cellular Component). FIG. 17G shows a Gene Ontology analysis of terms associated with genes upregulated in ALS oligodendrocytes, highlighted terms involved in neuro-supportive functions (CC=Cellular Component.

FIGS. 18A-18H provide a comparison of ALS-driven changes with other studies identified similar signatures disrupted in the disease. FIG. 18A provides a t-SNE projection and Violin plot representing z-score for genes characteristic of highly myelinating, OPALIN+ oligodendrocytes in Jäkel et al. FIG. 18B provides a Violin plot showing OPALIN expression in the dataset. FIG. 18C provides a t-SNE projection and Violin plot representing z-score for genes of mature, not-actively myelinating oligodendrocytes in Jake′ et al. FIG. 18D provides a Violin plot showing DLG1 expression in the dataset. FIG. 18E shows a comparison of genes downregulated in oligodendroglia from ALS patients with genes characteristic of highly myelinating, OPALIN+ subtypes identified by this study (oliglia0) and by Jake′ et al (Jäkel6), highlighted genes are shared with GO terms shown in figures. FIGS. 18F-18G show a comparison of genes upregulated in oligodendroglia from ALS patients with genes characteristic of mature, lowly myelinating groups in this study (oliglial and 4) and by Jake′ et al (Jäkel1), highlighted genes are shared with GO terms shown in figures. FIG. 18H provides a Dotplot representing z-scores for the genetic signatures identified in the actively myelinating cells, the mature lowly myelinating cells and DEGs identified in this study.

FIGS. 19A-19J demonstrate shared features between ALS-driven changes and reactive subcluster of microglia. FIG. 19A provides a t-SNE projection of microglia by individual. FIG. 19B provides a t-SNE projection of subclusters identified within microglia (Micro0=Homeo=homeostatic, Micro1=DAMs=Disease-associated microglia, Micro2=Cycling cells)). FIG. 19C shows a distribution of microglia within clusters by diagnosis. FIG. 19D shows a distribution of microglia within subclusters by individual. FIG. 19E provides a Dotplot representing genes identified as characteristic of Homeostatic microglia and DAMs by subcluster. FIG. 19F provides a Dotplot representing genes identified as characteristic of Homeostatic microglia and DAMs by diagnosis. FIG. 19G provides a Volcano plot of statistically significant differentially expressed genes between Control and ALS microglia (top ten upregulated and top ten downregulated genes highlighted). FIG. 19H provides Violin plots of representative DEGs downregulated in ALS patients of genes associated with homeostatic microglia. FIG. 19I shows a Gene Ontology analysis of terms associated with genes characteristic of DAMs microglia, highlighted terms playing important role in microglial biology and/or pathogenesis of the disease. FIG. 19J provides t-SNE projections representing z-score for selected, statistically significant GO terms.

FIGS. 20A-20D demonstrate apoptotic neurons upregulate lysosomal genes in microglia. FIG. 20A provides a schematic of workflow and results from the Connectivity Map project for the genes upregulated in ALS microglia. Heatmap shows what cellular signature is most closely related to the query. FIG. 20B provides a diagram of microglia and neuronal differentiation from Pluripotent Stem Cells, induction of apoptosis neurons and feeding to iMGLs. FIG. 20C provides Brightfield images of untreated day 40 iMGLs and day 40 iMGLs fed apoptotic neurons for 24 hours. FIG. 20D shows RT-qPCR quantification of ALS-driven genes after feeding apoptotic neurons to iMGLs.

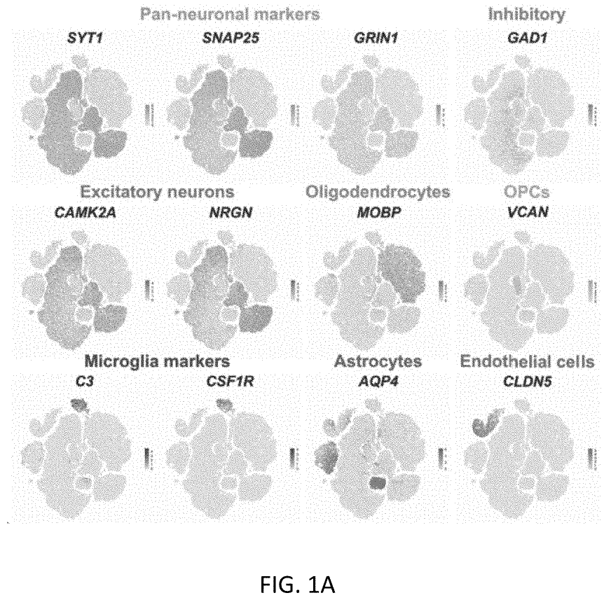

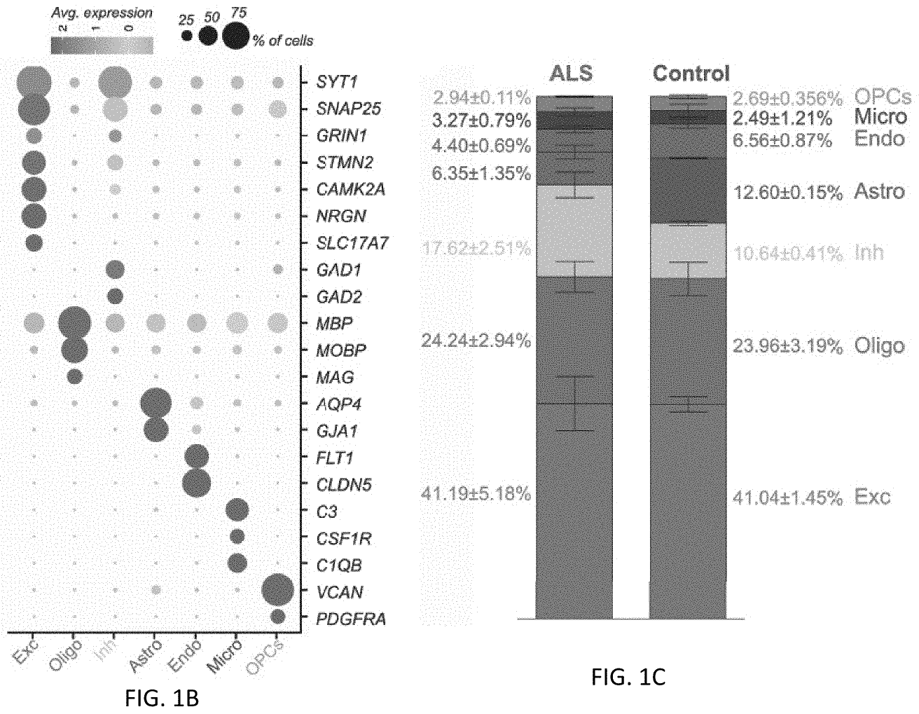

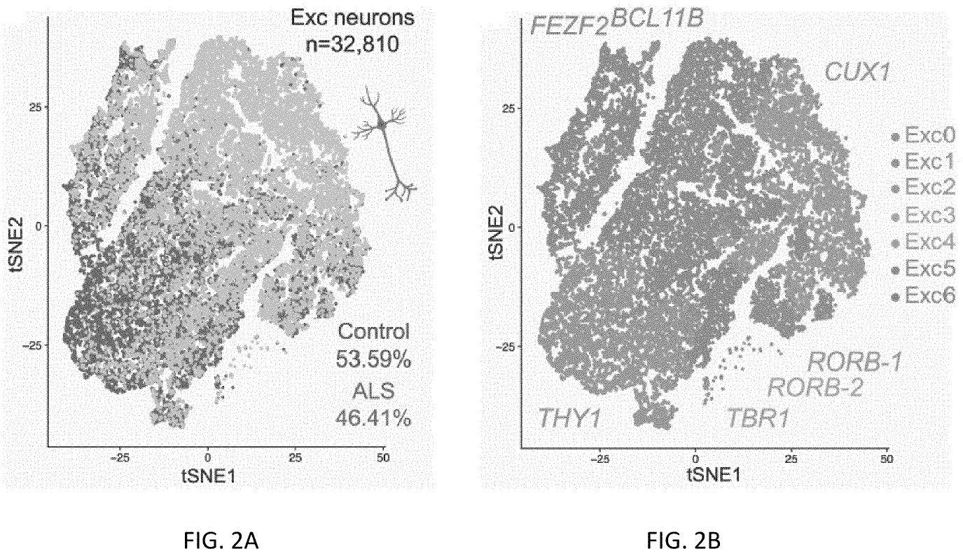

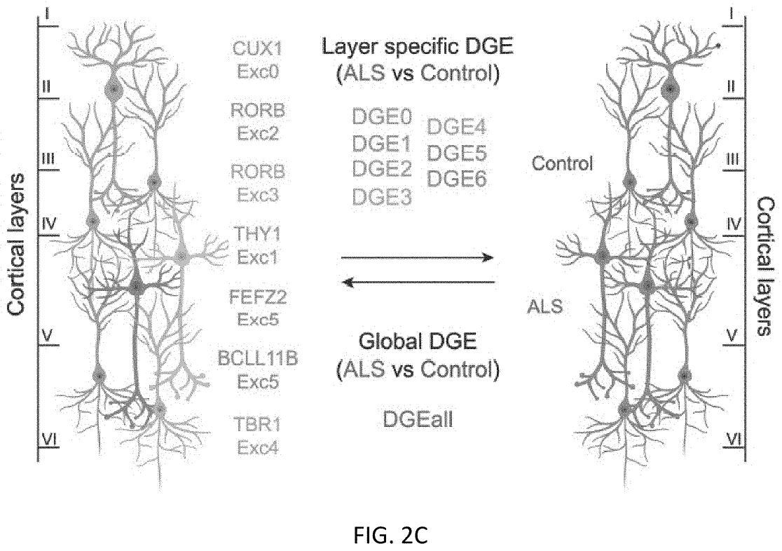

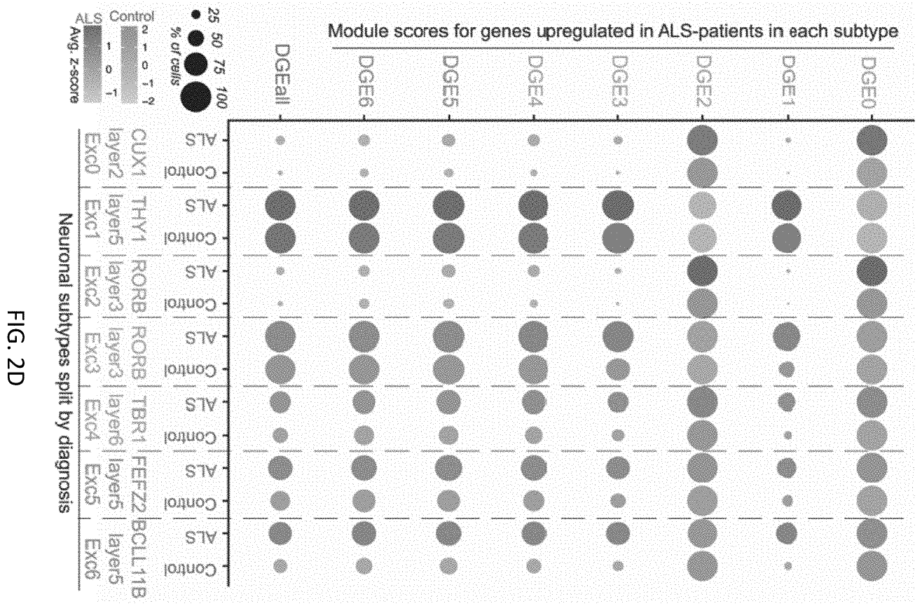

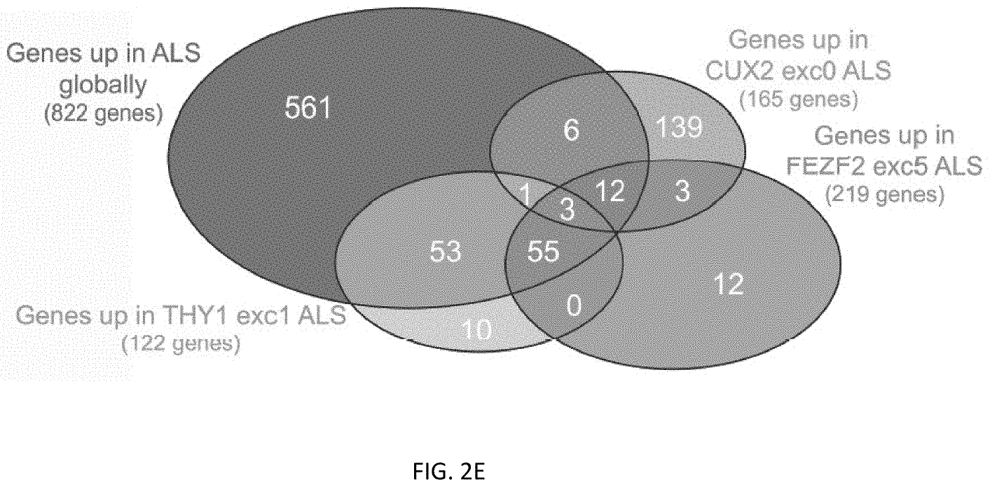

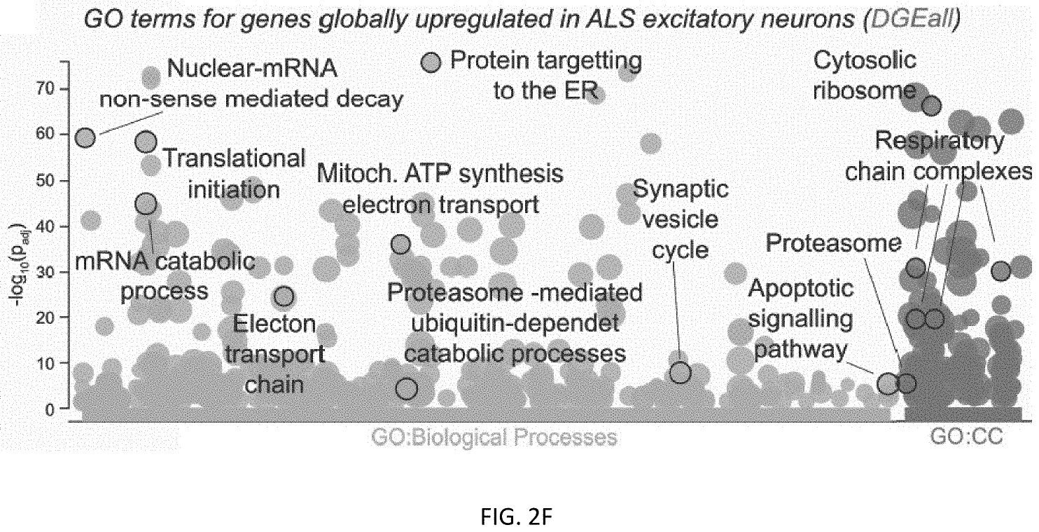

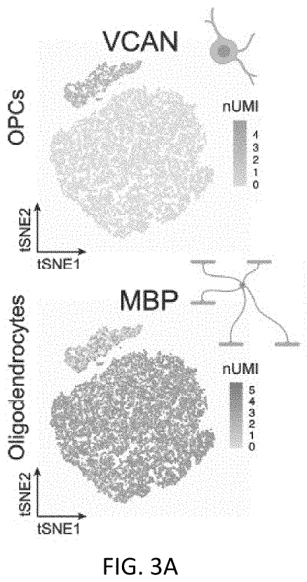

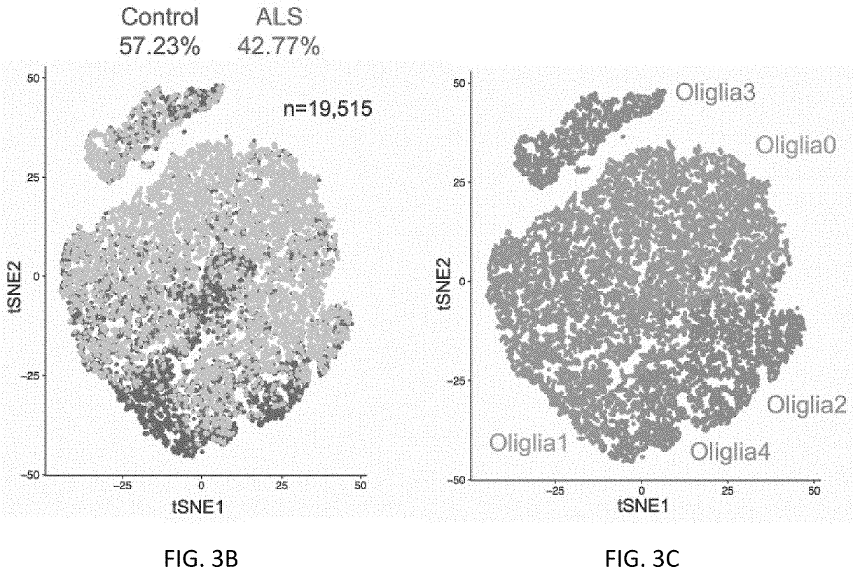

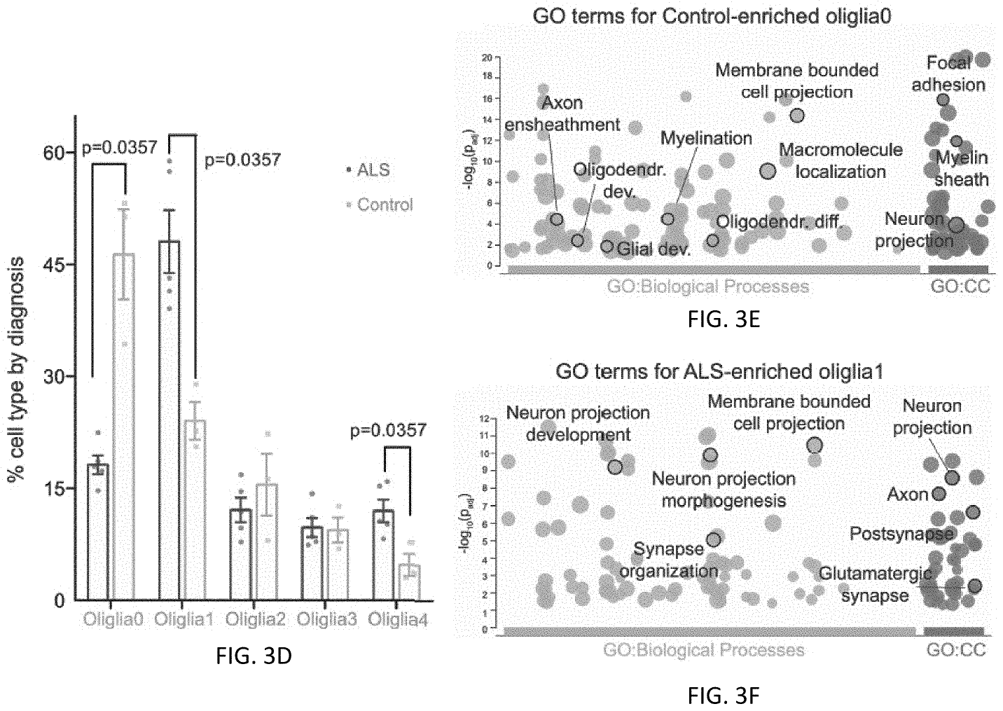

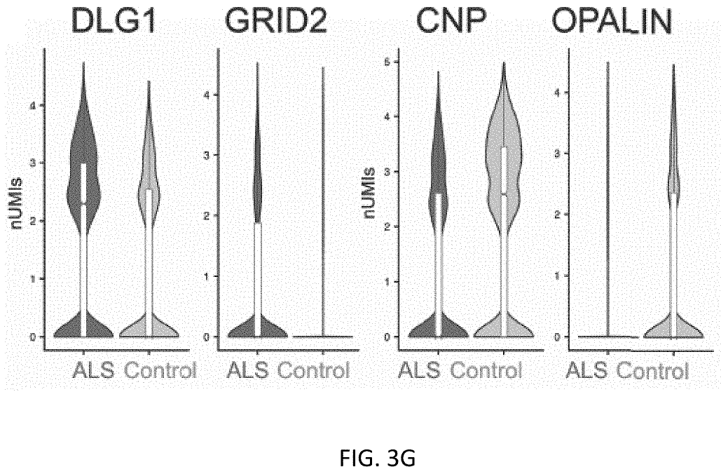

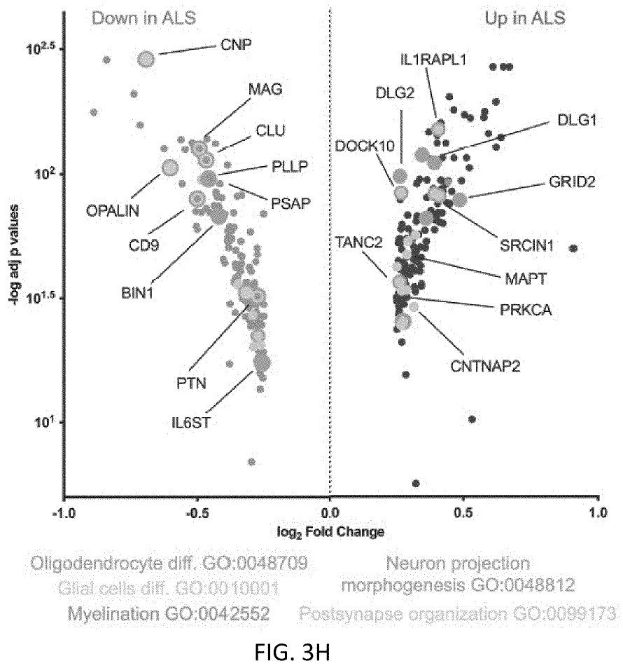

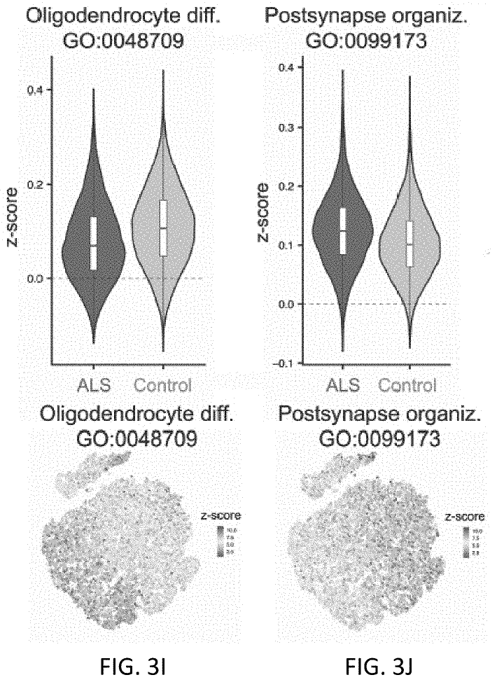

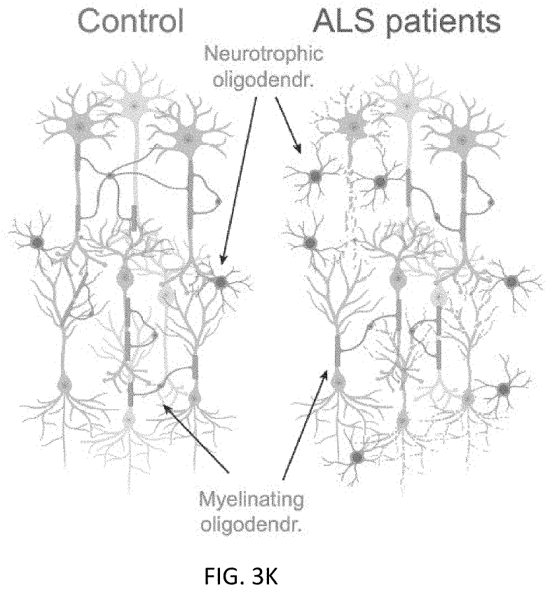

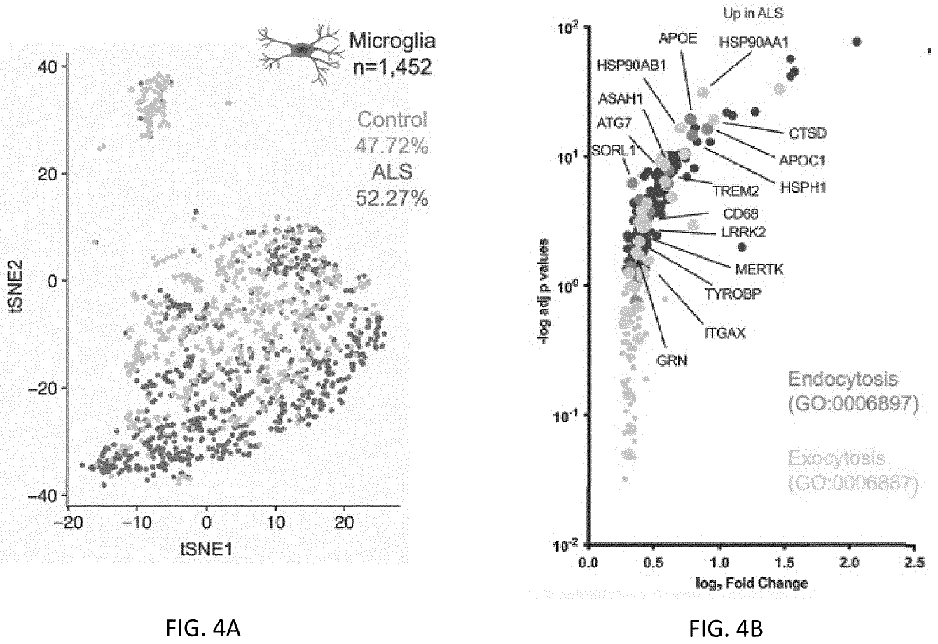

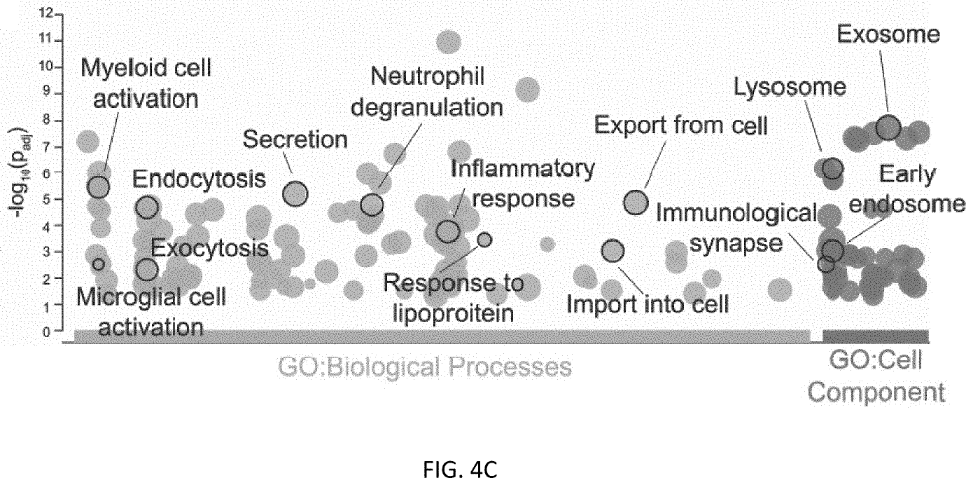

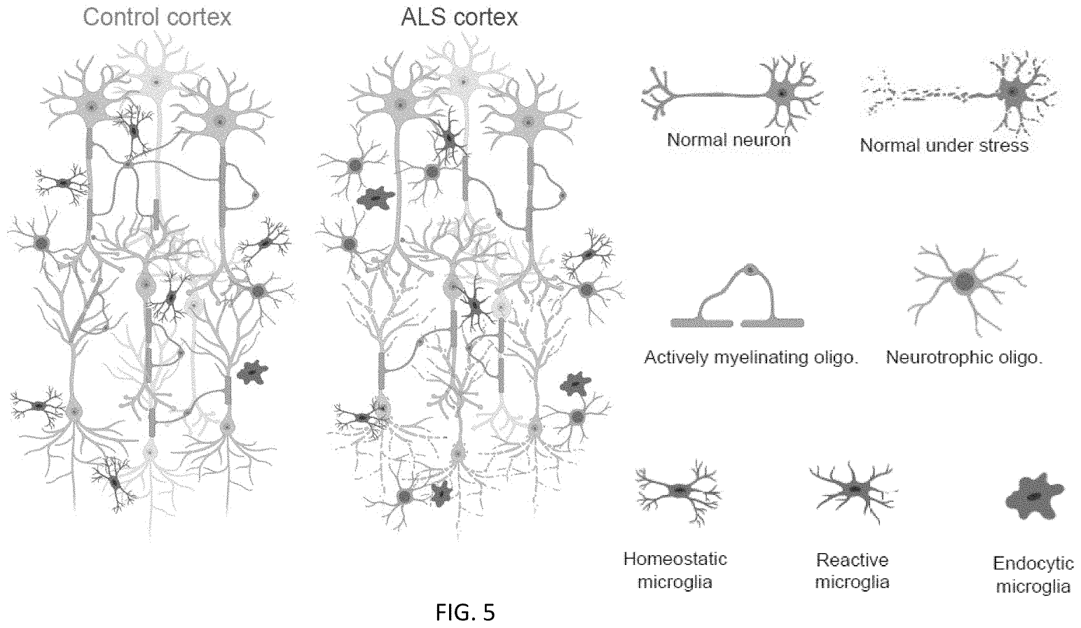

### DETAILED DESCRIPTION OF THE INVENTION

Amyotrophic Lateral Sclerosis (ALS) is a fatal neurodegenerative disorder characterized by a progressive loss of motor function. While it is known that the eponymous spinal sclerosis observed upon autopsy is the result of Cortico-Spinal Motor Neuron (CSMN) degeneration, it remains unclear why this neuronal subtype is selectively affected. To understand the unique molecular properties that sensitize deep-layer CSMNs to ALS, RNA sequencing of 79,169 single nuclei from the frontal cortex of patients and controls was performed. In unaffected individuals, it was found that expression of ALS risk genes was most significantly enriched only in THY1⁺ presumptive CSMNs and not in other cortical cell types. In patients, these genetic risk factors, as well as additional genes involved in protein homeostasis and stress responses, were significantly induced in THY1⁺ CSMNs and a wider collection of deep layer neurons, but not in neurons with more superficial identities. Examination of oligodendroglial and microglial nuclei also revealed patient-specific gene expression changes. It was shown that microglial alterations can in part be explained by interactions with degenerating neurons. Overall, the findings suggest the selective vulnerability of CSMNs is due to a “first over the line” mechanism by which their intrinsic molecular properties sensitize them to genetic and mechanistic contributors to degeneration.

Described herein are methods of treating a neurological disease or disorder in a subject in need thereof. In some embodiments, an agent is administered to a subject suffering from a neurological disease or disorder. In some embodiments, the neurological disease or disorder is a neurodegenerative disease or disorder, e.g., amyotrophic lateral sclerosis (ALS) and/or frontotemporal disorder (FTD).

“Neurodegenerative disorder” refers to a disease condition involving neural loss mediated or characterized at least partially by at least one of deterioration of neural stem cells and/or progenitor cells. Non-limiting examples of neurological diseases and/or disorders of the present disclosure include polyglutamine expansion disorders (e.g., HD, dentatorubropallidoluysian atrophy, Kennedy's disease (also referred to as spinobulbar muscular atrophy), and spinocerebellar ataxia (e.g., type 1, type 2, type 3 (also referred to as Machado-Joseph disease), type 6, type 7, and type 17)), other trinucleotide repeat expansion disorders (e.g., fragile X syndrome, fragile XE mental retardation, Friedreich's ataxia, myotonic dystrophy, spinocerebellar ataxia type 8, and spinocerebellar ataxia type 12), Alexander disease, Alper's disease, Alzheimer disease, amyotrophic lateral sclerosis (ALS), ataxia telangiectasia, Batten disease (also referred to as Spielmeyer-Vogt-Sjogren-Batten disease), Canavan disease, Cockayne syndrome, corticobasal degeneration, Creutzfeldt-Jakob disease, Guillain-Barré syndrome, ischemia stroke, Krabbe disease, kuru, Lewy body dementia, multiple sclerosis, multiple system atrophy, non-Huntingtonian type of Chorea, Parkinson's disease, Pelizaeus-Merzbacher disease, Pick's disease, primary lateral sclerosis, progressive supranuclear palsy, Refsum's disease, Sandhoff disease, Schilder's disease, spinal cord injury, spinal muscular atrophy (SMA), SteeleRichardson-Olszewski disease, schizophrenia, late onset psychosis, autism spectrum disorder, a movement disorder, and Tabes dorsalis. In some contexts neurodegenerative disorders encompass neurological injuries or damages to the CNS or PNS associated with physical injury (e.g., head trauma, mild to severe traumatic brain injury (TBI), diffuse axonal injury, cerebral contusion, acute brain swelling, and the like).

In some embodiments the neurodegenerative disorder is a disorder that is associated with mutant or reduced levels of TDP-43 in neuronal cells. In some embodiments, the neurological disease is amyotrophic lateral sclerosis (ALS). In some embodiments, the neurological disease is sporadic ALS. In some embodiments, the neurological disease is familial ALS. In some embodiments, the neurological disease comprises multiple sclerosis (MS) and/or Alzheimer's disease (AD).

A neurological disease or disorder described herein may be characterized by increased expression of genetic risk factors for ALS/FTD in cortico-spinal motor neurons (CSMNs) (e.g., THY1⁺ CSMNs). In some embodiments, the neurological disease or disorder is characterized by one or more of an oligodendroglia shift from a myelinating to a neuronally-engaged state, superficial neurons upregulating synaptic genes, and activation of a pro-inflammatory state by microglia in response to neuronal degeneration. In some embodiments, the neurological disease or disorder may be characterized by transcriptional perturbations in endo-lysosomal pathways. In some aspects, the neurological disease or disorder is characterized by upregulated expression of genes involved in neuro-supportive functions and/or downregulated expression of genes involved in myelination.

In some embodiments, the agent modulates neuronal regeneration. In some embodiments, the agent increases the uptake of toxic proteins from the intercellular environment. In some embodiments, the agent increases expression of Sorillin-like-1 protein (SORL1). In some embodiments, the agent increases expression of SORL1 in microglia. In some embodiments, the agent increases expression of SORL1 in neurons. In some embodiments, the agent increases expression of SORL1 in microglia and neurons. In some embodiments, the methods of treatment comprise increasing expression of SORL1, e.g., in microglia and/or neurons.

The terms “increased” or “increase” are used herein to generally mean an increase by a statically significant amount; for the avoidance of any doubt, the terms “increased”, or “increase” means an increase of at least 10% as compared to a reference level, for example an increase of at least about 20%, or at least about 30%, or at least about 40%, or at least about 50%, or at least about 60%, or at least about 70%, or at least about 80%, or at least about 90%, or up to and including a 100% increase or any increase between 10-100% as compared to a reference level, or at least about a 2-fold, or at least about a 3-fold, or at least about a 4-fold, or at least about a 5-fold, or at least about a 10-fold increase, or any increase between 2-fold and 10-fold or greater as compared to a reference level.

In some embodiments, the agent modulates (e.g., reduces) toxicity of the inhibited proteasome. In some embodiments, the agent increases proteasome activity by reducing the inhibitory activity of a proteasome subunit (e.g., PSMD12). In some embodiments, the agent protects neurons from proteasome inhibition. In some embodiments, the agent decreases expression of 26S Proteasome Non-ATPase Regulatory Subunit 12 (PSMD12). In some embodiments, the agent decreases expression of PSMD12 in neurons. In some embodiments, the methods of treatment comprise administering an effective amount of an agent that decreases expression of PSMD12, e.g., in neurons.

The terms “decrease,” “reduce,” “reduced,” “reduction,” “decrease,” and “inhibit” are all used herein generally to mean a decrease by a statistically significant amount relative to a reference. However, for avoidance of doubt, “reduce,” “reduction” or “decrease” or “inhibit” typically means a decrease by at least 10% as compared to a reference level and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the given entity or parameter as compared to the reference level, or any decrease between 10-99% as compared to the absence of a given treatment.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In some embodiments, the agent is selected from the group consisting of a nucleic acid, a small molecule, a polypeptide, and a peptide. In some embodiments the agent is an oligonucleotide, protein, or a small molecule. In some embodiments the agent comprises one or more oligonucleotides. In some aspects the oligonucleotide is a splice-switching oligonucleotide. In certain aspects the oligonucleotide is an antisense oligonucleotide (ASO). In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds. In some embodiments, the agent is a genomic modification system (e.g., a CRISPR/Cas, Zinc Finger Nuclease, or TALEN systems). CRISPR/Cas systems can employ a variety of Cas proteins (Haft et al. PLoS Comput Biol. 2005; 1(6)e60). In some embodiments, the CRISPR/Cas system is a CRISPR type I system. In some embodiments, the CRISPR/Cas system is a CRISPR type II system. In some embodiments, the CRISPR/Cas system is a CRISPR type V system.

“Small molecule” is defined as a molecule with a molecular weight that is less than 10 kD, typically less than 2 kD, and preferably less than 1 kD. Small molecules include, but are not limited to, inorganic molecules, organic molecules, organic molecules containing an inorganic component, molecules comprising a radioactive atom, synthetic molecules, peptide mimetics, and antibody mimetics. As a therapeutic, a small molecule may be more permeable to cells, less susceptible to degradation, and less apt to elicit an immune response than large molecules.

As used herein, the term “polypeptide” or “protein” is used to designate a series of amino acid residues connected to the other by peptide bonds between the alpha-amino and carboxy groups of adjacent residues. The term “polypeptide” refers to a polymer of protein amino acids, including modified amino acids (e.g., phosphorylated, glycated, glycosylated, etc.) and amino acid analogs, regardless of its size or function. The term “peptide” is often used in reference to small polypeptides, but usage of this term in the art overlaps with “protein” or “polypeptide.” Exemplary polypeptides include gene products, naturally occurring proteins, homologs, orthologs, paralogs, fragments and other equivalents, as well as both naturally and non-naturally occurring variants, fragments, and analogs of the foregoing.

The term “nucleic acid” refers to polynucleotides such as deoxyribonucleic acid (DNA) and ribonucleic acid (RNA). The terms “nucleic acid” and “polynucleotide” are used interchangeably herein and should be understood to include double-stranded polynucleotides, single-stranded (such as sense or antisense) polynucleotides, and partially double-stranded polynucleotides. A nucleic acid often comprises standard nucleotides typically found in naturally occurring DNA or RNA (which can include modifications such as methylated nucleobases), joined by phosphodiester bonds. In some embodiments a nucleic acid may comprise one or more non-standard nucleotides, which may be naturally occurring or non-naturally occurring (i.e., artificial; not found in nature) in various embodiments and/or may contain a modified sugar or modified backbone linkage. Nucleic acid modifications (e.g., base, sugar, and/or backbone modifications), non-standard nucleotides or nucleosides, etc., such as those known in the art as being useful in the context of RNA interference (RNAi), aptamer, CRISPR technology, polypeptide production, reprogramming, or antisense-based molecules for research or therapeutic purposes may be incorporated in various embodiments. Such modifications may, for example, increase stability (e.g., by reducing sensitivity to cleavage by nucleases), decrease clearance in vivo, increase cell uptake, or confer other properties that improve the translation, potency, efficacy, specificity, or otherwise render the nucleic acid more suitable for an intended use. Various non-limiting examples of nucleic acid modifications are described in, e.g., Deleavey G F, et al., Chemical modification of siRNA. Curr. Protoc. Nucleic Acid Chem. 2009; 39:16.3.1-16.3.22; Crooke, S T (ed.) Antisense drug technology: principles, strategies, and applications, Boca Raton: CRC Press, 2008; Kurreck, J. (ed.) Therapeutic oligonucleotides, RSC biomolecular sciences. Cambridge: Royal Society of Chemistry, 2008; U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929, 226; 5,977,296; 6,140,482; 6,455,308 and/or in PCT application publications WO 00/56746 and WO 01/14398. Different modifications may be used in the two strands of a double-stranded nucleic acid. A nucleic acid may be modified uniformly or on only a portion thereof and/or may contain multiple different modifications. Where the length of a nucleic acid or nucleic acid region is given in terms of a number of nucleotides (nt) it should be understood that the number refers to the number of nucleotides in a single-stranded nucleic acid or in each strand of a double-stranded nucleic acid unless otherwise indicated. An “oligonucleotide” is a relatively short nucleic acid, typically between about 5 and about 100 nt long.

In some embodiments, the subject is also administered a second agent to treat or prevent a neurological disease or disorder. In some embodiments, the first and second agent are co-formulated. In some embodiments, the first and second agent are administered simultaneously. In some embodiments, the first and second agent are administered within a time of each other to produce overlapping therapeutic effects in the patient. When the first and second agent are administered simultaneously or within a time of each other to produce overlapping therapeutic effects, the agents may be administered by the same or a different route of administration (e.g., oral versus infusion).

For administration to a subject, the agents disclosed herein can be provided in pharmaceutically acceptable compositions. These pharmaceutically acceptable compositions comprise a therapeutically-effective amount of one or more of the agents, formulated together with one or more pharmaceutically acceptable carriers (additives) and/or diluents. The pharmaceutical compositions of the present invention can be specially formulated for administration in solid or liquid form, including those adapted for the following: (1) oral administration, for example, drenches (aqueous or non-aqueous solutions or suspensions), gavages, lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intrathecal, intercranially, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, agents can be implanted into a patient or injected using a drug delivery system. (See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960, content of all of which is herein incorporated by reference.)

As used herein, the term “pharmaceutically acceptable” refers to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject agent from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

The phrase “therapeutically-effective amount” as used herein means that amount of an agent, material, or composition comprising an agent described herein which is effective for producing some desired therapeutic effect in at least a sub-population of cells in an animal at a reasonable benefit/risk ratio applicable to any medical treatment. For example, an amount of an agent administered to a subject that is sufficient to produce a statistically significant, measurable decrease in the expression of PSMD12. In another example, an amount of an agent administered to a subject that is sufficient to produce a statistically significant, measurable increase in the expression of SORL1.

The determination of a therapeutically effective amount of the agents and compositions disclosed herein is well within the capability of those skilled in the art. Generally, a therapeutically effective amount can vary with the subject's history, age, condition, sex, and the administration of other pharmaceutically active agents.

As used herein, the term “administer” refers to the placement of an agent or composition into a subject (e.g., a subject in need) by a method or route which results in at least partial localization of the agent or composition at a desired site such that desired effect is produced. Routes of administration suitable for the methods of the invention include both local and systemic routes of administration. Generally, local administration results in more of the administered agents being delivered to a specific location as compared to the entire body of the subject, whereas, systemic administration results in delivery of the agents to essentially the entire body of the subject.

The compositions and agents disclosed herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration. Exemplary modes of administration include, but are not limited to, injection, infusion, instillation, inhalation, or ingestion. “Injection” includes, without limitation, intravenous, intramuscular, intraarterial, intrathecal, intraventricular, intracranial, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments of the aspects described herein, the compositions are administered by intravenous infusion or injection.

As used herein, a “subject” means a human or animal (e.g., a mammal). Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human. The terms, “patient” and “subject” are used interchangeably herein. A subject can be male or female.

As used herein, “treat,” “treatment,” or “treating” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state of, for example, a neurodegenerative disorder, delay or slowing progression of a neurodegenerative disorder, and an increased lifespan as compared to that expected in the absence of treatment.

The disclosure further contemplates pharmaceutical compositions comprising an agent that treats a neurological disease or disorder. In some embodiments, the pharmaceutical composition comprises an oligonucleotide (e.g., an antisense oligonucleotide), protein, small molecule, antibody, siRNA, and/or gene therapy (e.g., CRISPR/Cas system). In some embodiments, the pharmaceutical composition comprises at least one agent and a pharmaceutically acceptable carrier, diluent, or excipient.

In some embodiments, the pharmaceutical composition comprises an agent that modulates neuronal regeneration. In some embodiments, the pharmaceutical composition comprises an agent that increases the uptake of toxic proteins from the intercellular environment. In some embodiments, the pharmaceutical composition comprises an agent that increases expression of SORL1, e.g., in microglia and/or neurons. In some embodiments, the pharmaceutical composition comprises an effective amount of an agent that increase SORL1 expression and an effective amount of a second agent. In some aspects, the second agent is an agent that treats or inhibits a neurodegenerative disorder.

In some embodiments, the pharmaceutical composition comprises an agent that modulates proteasome inhibition toxicity. In some embodiments, the pharmaceutical composition comprises an agent that protects neurons from proteasome inhibition. In some embodiments, the pharmaceutical composition comprises an agent that decreases expression of PSMD12, e.g., in neurons. In some embodiments, the pharmaceutical composition comprises an effective amount of an agent that decreases PSMD12 expression and an effective amount of a second agent. In some aspects, the second agent is an agent that treats or inhibits a neurodegenerative disorder.

The disclosure further contemplates methods of screening one or more test agents to identify candidate agents for treating or reducing the likelihood of a neurological disease or disorder, e.g., a neurodegenerative disorder. In some embodiments, the methods comprise providing a neuronal cell expressing SORL1 (e.g., having decreased expression of SORL1 compared to a control cell); contacting the cell with one or more test agents; determining if the contacted cell has an increased expression level of SORL1; and identifying the test agent as a candidate agent if the contacted cell has an increased expression level of SORL1. In other embodiments, the methods comprise providing a neuronal cell expressing PSMD12 (e.g., having increased expression of PSMD12 compared to a control cell); contacting the cell with one or more test agents; determining if the contacted cell has a decreased expression level of PSMD12; and identifying the test agent as a candidate agent if the contacted cell has a decreased expression level of PSMD12.

In some embodiments, the step of determining if the contacted cell has increased levels of SORL1 expression comprises measuring SORL1 protein levels in the contacted cell. In some embodiments, the step of determining if the contacted cell has decreased levels of PSMD12 expression comprises measuring PSMD12 protein levels in the contacted cell. In some embodiments, the SORL1 and/or PSMD12 protein levels are measured using an ELISA assay. In some embodiments, the neurodegenerative disease or condition is selected from the group consisting of amyotrophic lateral sclerosis (ALS), frontotemporal dementia (FTD), Alzheimer's disease (AD), and multiple sclerosis (MS). In certain aspects, the neurodegenerative disease or condition is ALS.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more active agents, additives, ingredients, optional agents, types of organism, disorders, subjects, or combinations thereof, can be excluded. Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”.

“Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

It is to be understood that the inventions disclosed herein are not limited in their application to the details set forth in the description or as exemplified. The invention encompasses other embodiments and is capable of being practiced or carried out in various ways. Also, it is to be understood that the phraseology and terminology employed herein is for the purpose of description and should not be regarded as limiting.

While certain compositions and methods of the present invention have been described with specificity in accordance with certain embodiments, the following examples serve only to illustrate the methods and compositions of the invention and are not intended to limit the same.

### EXEMPLIFICATION

### Example 1

Described herein is a study that provides an increased resolution of the complex cellular landscape in sporadic ALS and underlying molecular pathways that contribute to this disease by profiling 79,830 droplet-based single nucleus cortical transcriptomes in sporadic ALS. In one instance, a specific vulnerability was identified of excitatory Cortico-Spinal Motor Neurons to perturbation driven by the disease. These changes seem to be closely related to cellular stress pathways often associated with the disease but not always with specific cellular subtypes. In another instance, it was found that the excitatory neurons vulnerability may be connected to a switch in oligodendroglial cells from a highly myelinating to a more neuro-supportive phenotype. Moreover, shared features and distinct differences were delineated between microglial cellular states found in ALS and those present in other neurological conditions, including AD.

A comprehensive and robust profile was provided of the complex cellular changes present in ALS at an unprecedented cellular resolution. The increased sensitivity of Drop-seq-based single-nuclei sequencing implicated a vast swath of cellular pathways across several disease-relevant cell types in human brain tissue that were not previously detected through other transcriptomic approaches, including bulk RNA-seq. It was demonstrated that this technique can be used to define subtle transcriptional changes in neuronal, glial, and vascular cell types in an unbiased manner without requiring prior cell-based sorting methods to enrich for a particular cell type.

Strikingly, distinct transcriptional perturbations and cellular heterogeneity were found among ALS associated microglia. snRNA-seq analysis demonstrated microglia clusters analogous to the previously defined homeostatic, DAM, and cycling subtypes. Importantly, substantial disease specific microglia transcriptional changes were identified that were present in a subset of “homeostatic” microglia from patients. This suggests an intermediate cellular state characterized by the activation of inflammatory and/or phagocytic responses that may proceed the homeostatic-to-DAM microglial transition in ALS-FTD. Moreover, the upregulation of genes involved with microglial phagocytic and inflammatory responses that are also associated with familial forms of the disease (TREM2, OPTN, SQSTM1, GRN) may initiate and/or exacerbate this transition. Overall, the top differentially expressed transcripts in microglia had partial overlap with disease-associated microglia surrounding amyloid plaques in Alzheimer's disease as well as microglial clusters associated with demyelinating lesions in multiple sclerosis (MS), suggesting that drugs specifically modulating microglia to restore homeostatic phenotypes in these diseases may also provide a basis for a new therapeutic approach for ALS.

Emerging studies have shown that glial cells are important modifiers of disease progression in animal models of familial ALS. For instance, removing toxic SOD1 protein from the oligodendrocyte lineage improves survival in the SOD1 G93A ALS mouse model, suggesting that dysfunction of oligodendrocytes and OPCs may contribute to motor neuron degeneration. In this study, several oligodendrocyte cellular subtypes were defined and it was demonstrated that changes in processes involved in myelination, oligodendrocyte differentiation, synapse organization, and neurotrophic support may contribute to neural degeneration or be a coordinating conjunction to a healthy response to the disease. This shift between myelinating and neuronal support has been identified in snRNA-seq studies in Multiple Sclerosis (Jake′ et al). Interestingly though oligodendrocytes in MS show an increase in myelination abilities and a loss in neurosupportive properties, suggesting a new role for oligodendrocytes as modulators of neuronal excitability ALS. Moreover, snRNA-seq analysis was able to uncover significant perturbations in key myelin-related genes, including OPALIN, CNP, and MAG, across multiple oligodendrocyte cellular clusters. In snRNA-seq of Alzheimer's disease, myelination related perturbations were detected across multiple cell types. In contrast, oligodendroglia specific changes were found that were not present in other cell types. These observations are also consistent with the dysregulation of oligodendrocyte maturation as a mechanism for how alterations in these glial cells could affect neuronal function and may aid in the design of therapies aiming to bolster oligodendrocyte differentiation.

The advances in snRNA-seq technology enhanced the ability to detect subtle molecular changes in specific cell type. This allowed the identification of a specific, ALS-driven cellular stress signature in excitatory neurons, specifically of the lower layers of the cortex. Many of these pathways involved in RNA-translation, proteostasis and mitochondrial function have often been associated with ALS-FTD, but this study identified them as specific to this cellular subtype. These findings underline the central role that neuro-centric cellular stress might play in the initiation of the disease. However, taken together with the changes identified in oligodendrocytes and microglial cells, this study underlines the role of non-cell autonomous pathways that might be exacerbators of the disease if not an additional mechanism of disease initiation and strengthens the importance of the disruptive/supportive roles of the different components of the cerebral milieu.

In addition, the cellular distribution of ALS associated genes among different cell types was identified. For instance, SOD1 and UBQLN2 had the highest relative expression in excitatory neuron expression. In contrast to prior reports that suggested C9ORF72 may be more abundant in myeloid-derived microglia, the results demonstrate that C9ORF72 and TBK1, which are both involved in autophagy pathways, are more broadly expressed among neuronal and non-neuronal cells. Hence, this resource will aid in the selection of relevant cell types for the further molecular dissection of ALS-related pathways.

Single nuclei RNAseq was used to analyze expression of genes associated with sporadic ALS/FTD in various whole cortex cell types. A strong signal of cell stress was shown to be activated in excitatory neurons, specifically in disease-relevant subtypes (e.g., CSMNs). In addition, a decrease in myelinating machinery of oligodendroglial cells in favor of a more neurotrophic state was identified. Further, it was shown that the microglial upregulates a strong reactive state connected to the endolysosome system. This work provides targets for therapeutic intervention for the treatment of ALS, particularly sporadic ALS, and FTD.

### Example 2—Single-Nucleus Sequencing Reveals Enriched Expression of Genetic Risk Factors Sensitizes Motor Neurons to Degeneration in ALS

Amyotrophic Lateral Sclerosis (ALS) is characterized by the selective degeneration of both cortical-spinal and spinal motor neurons¹. Although specific genetic causes of ALS have been identified, most cases are sporadic and have no family history of disease²³. Bulk RNA-sequencing of post-mortem brain tissues has begun to identify gene expression alterations in both sporadic and familial forms of the disease⁴⁻⁷. One likely contributor of these alterations in gene expression is the aggregation and nuclear clearance of TAR DNA-binding protein-43 (TDP-43), which is found in the brain and spinal cord of over 95% of cases⁸. However, the tissue level gene-expression analysis that has been reported to date has left uncertainty concerning the way in which distinct subtypes of neurons, including cortico-spinal motor neurons (CSMNs), are altered in the disease. Furthermore, it is increasingly understood that non-neuronal, glial cells are important modulators of neuronal degeneration, but it remains unclear how transcripts in these cell types are modulated in ALS⁹⁻¹².

Methods to measure transcript abundance at a single-cell level have rapidly advanced and their application to nuclei from human post-mortem brain tissue has provided new insights into how individuals brain cell types are altered in Multiple Sclerosis (MS)^{13,14} and Alzheimer's disease (AD)^{15,16}. Here, findings from RNA sequencing of single nuclei isolated from sporadic ALS and control pre-frontal cortex are reported. Analyses of these data identify pathways altered by ALS in individual classes of cells and suggest a molecular explanation for the selective sensitivity of corticospinal motor neurons to degeneration.

### Profiling of ALS Frontal Cortex by Single-Nucleus RNA-Sequencing

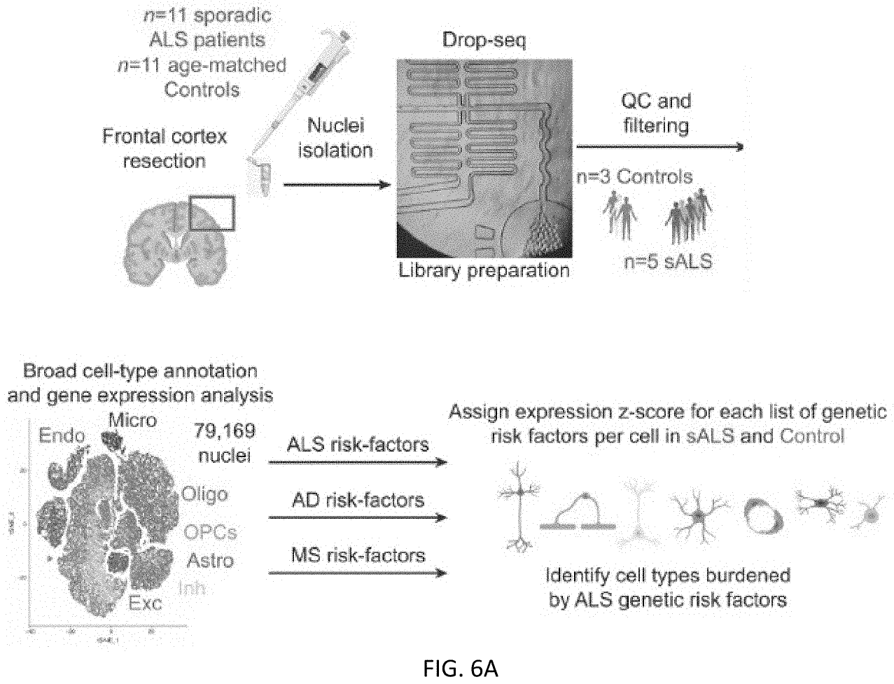

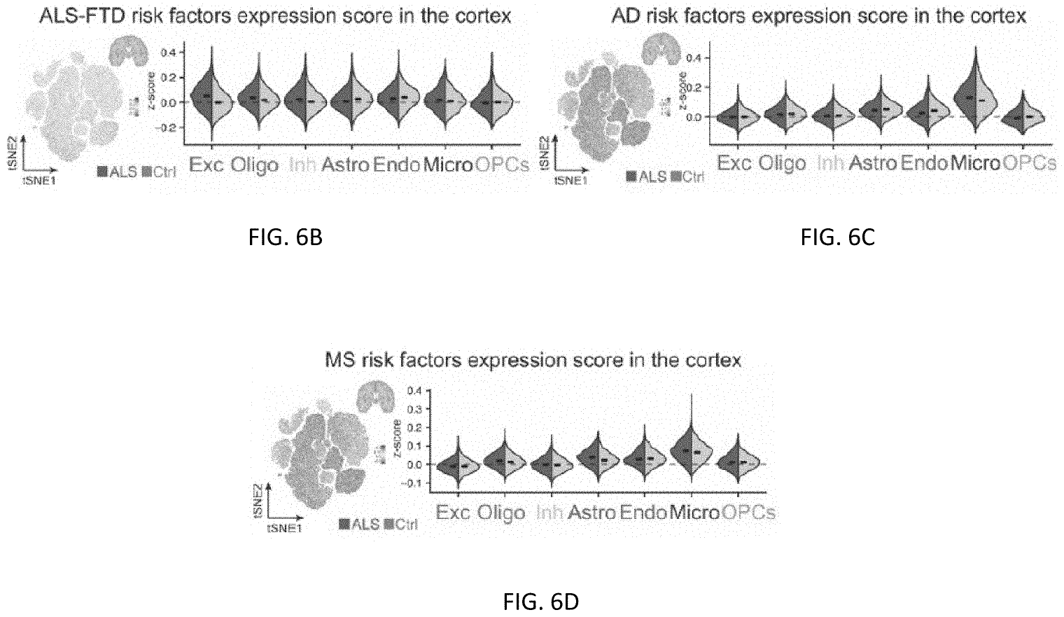

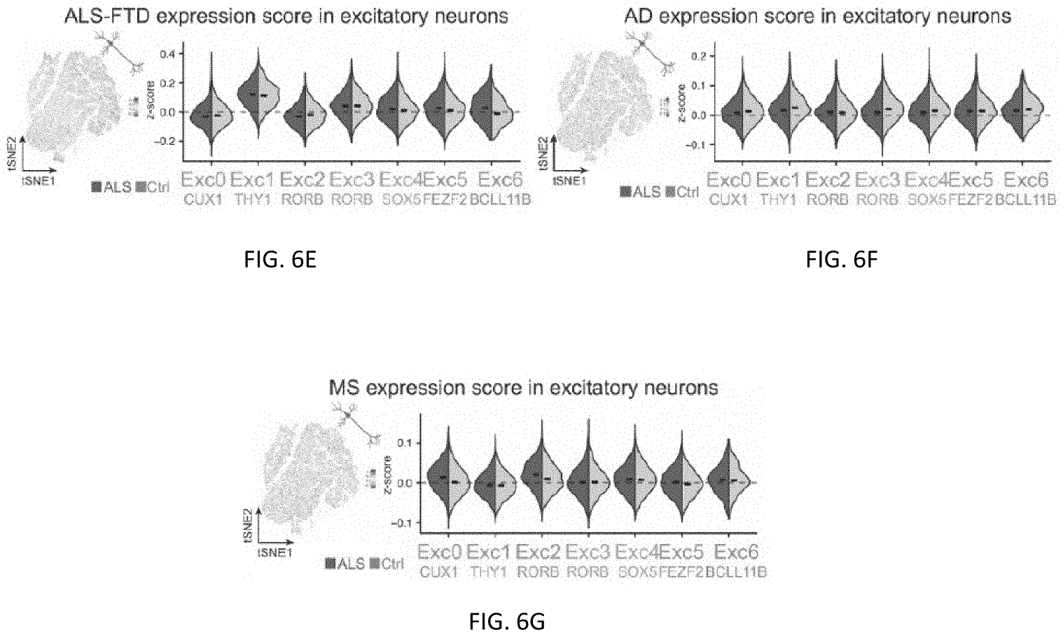

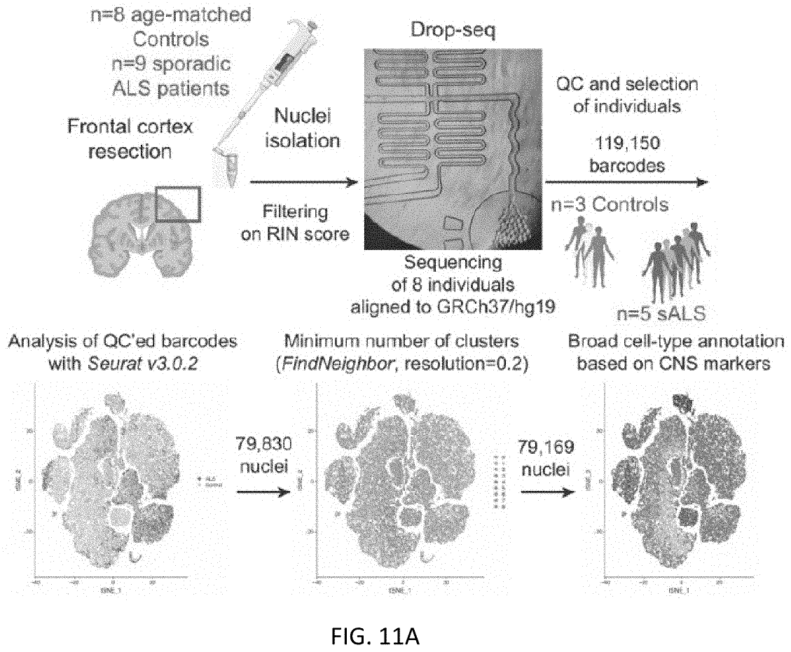

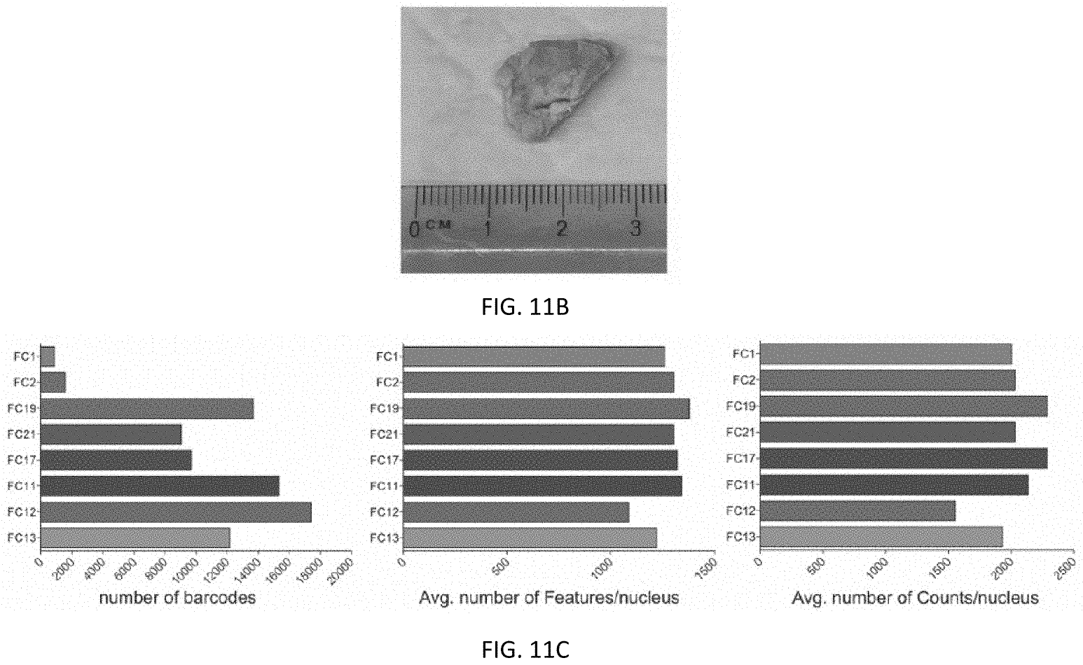

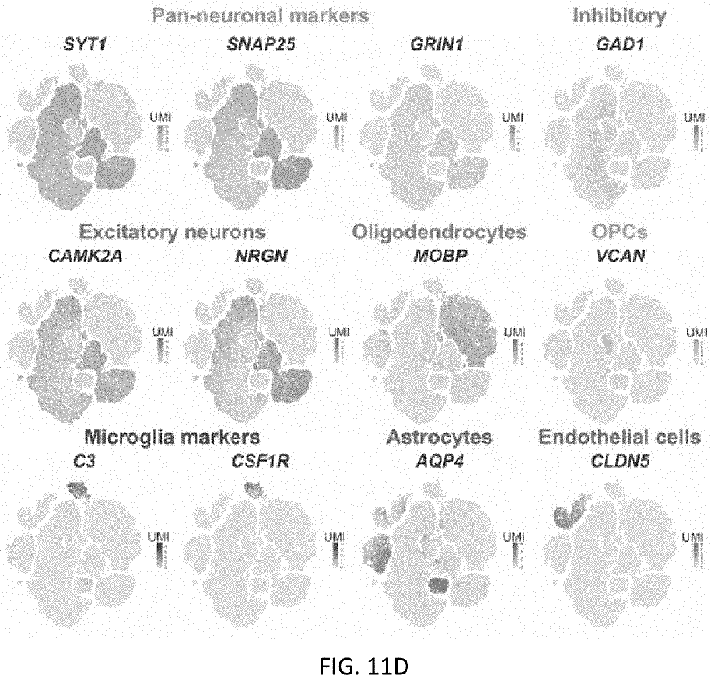

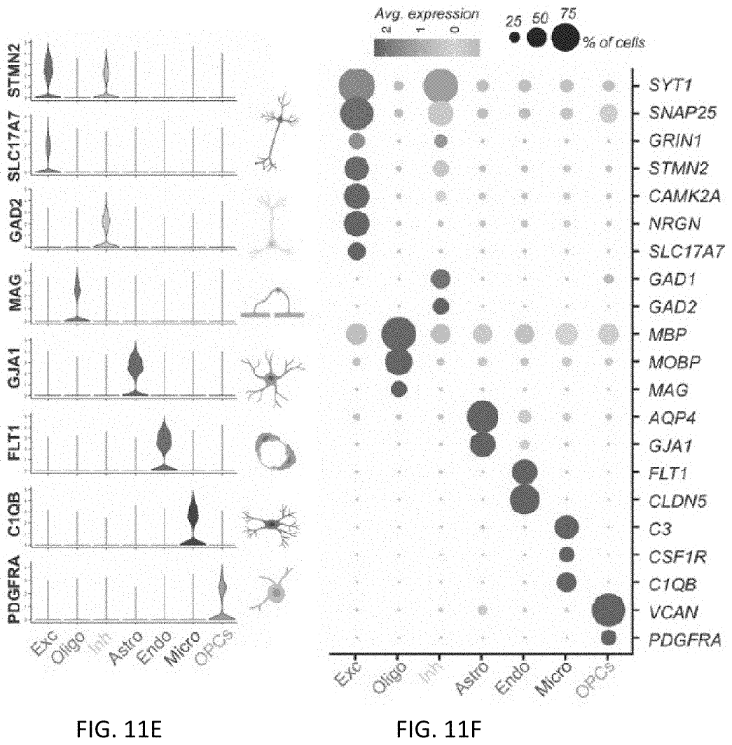

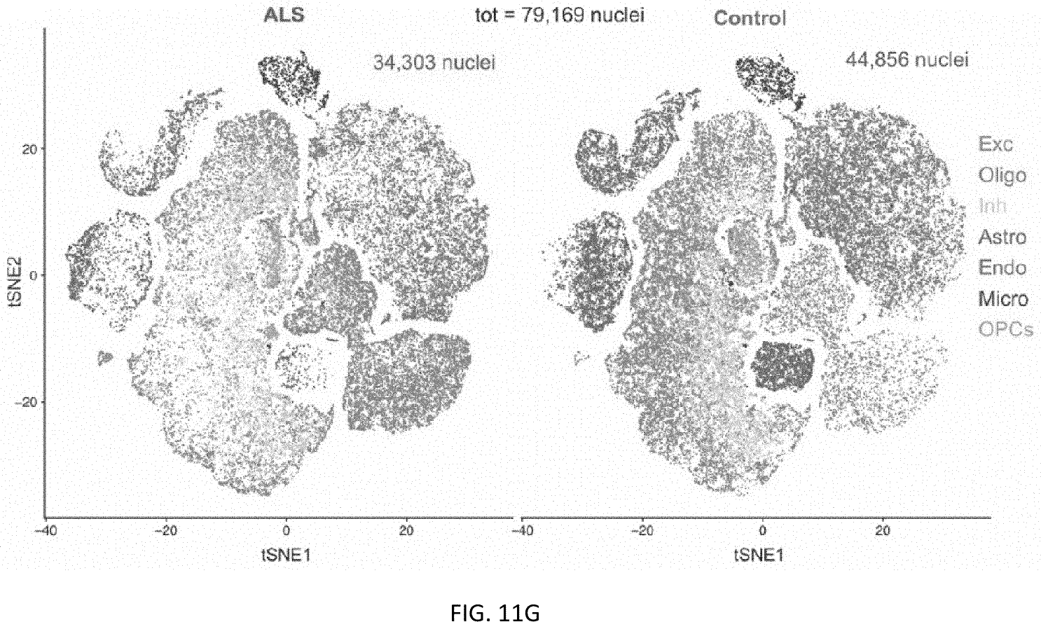

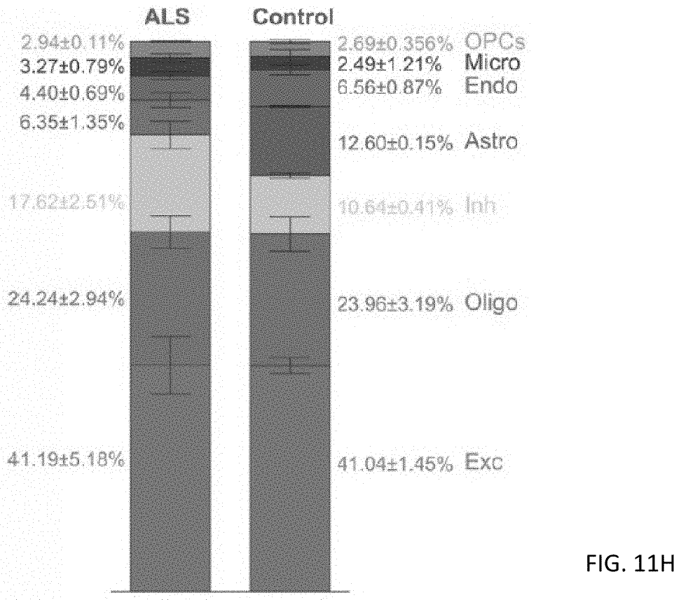

To better understand factors that might contribute to the specific degeneration of classes of deep layer excitatory neurons, including CSMNs, single nucleus RNA sequencing was used to profile frontal cortex grey matter from 9 sporadic (sALS) patients and 8 age-matched controls with no known neurological disease using Drop-seq¹⁷. After screening for RNA quality, barcoded libraries from 119,510 individual nuclei, from 8 individuals were analyzed (n=5 sALS, n=3 Control) (FIG. 6A). Further quality control yielded 79,169 nuclear libraries (barcodes) with a mean of 1269 genes and 2026 unique molecular identifiers (UMIs) (FIGS. 11A-11C). Seurat¹⁸, a single-cell analysis R package, was used to cluster and annotate nuclear libraries according to canonical markers of brain cell types: excitatory and inhibitory neurons, oligodendrocytes, oligodendrocyte progenitor cells (OPCs), microglia, astrocytes, and endothelial cells (FIGS. 11D-11F). The observed cell type distribution corresponded to previous studies¹⁹ and enabled robust categorization for downstream analysis. The cellular distribution was homogeneous between sexes and individuals, except for a modest decrease in the number of astrocytes in ALS samples (FIGS. 11G-11H).

### Elevated Expression of ALS-FTD Risk Genes in a Specific Class of CSMNs

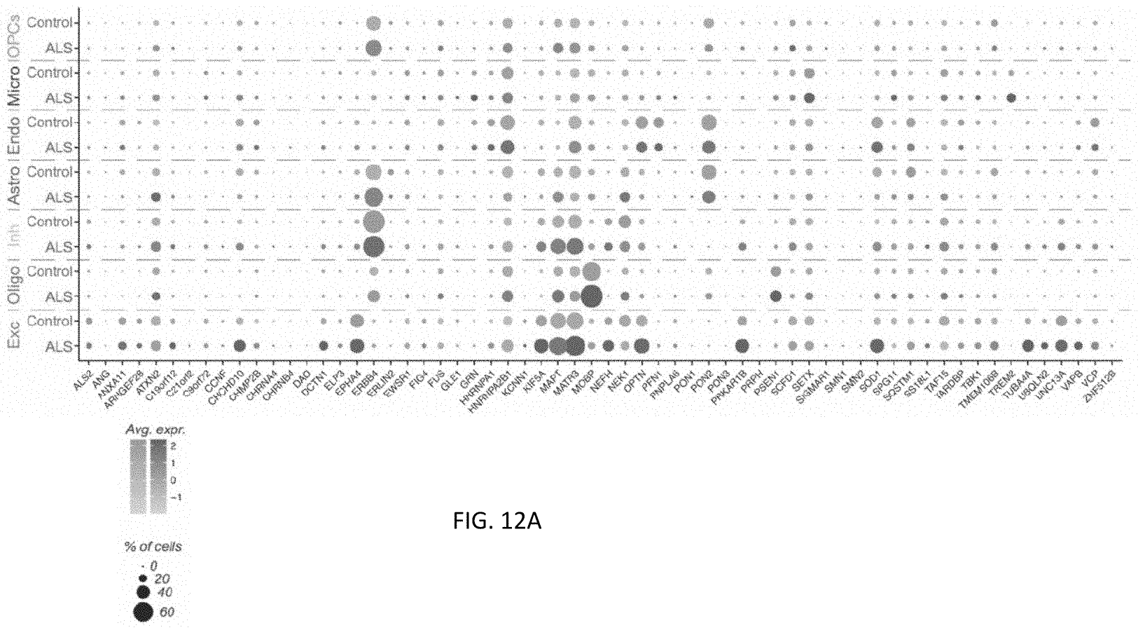

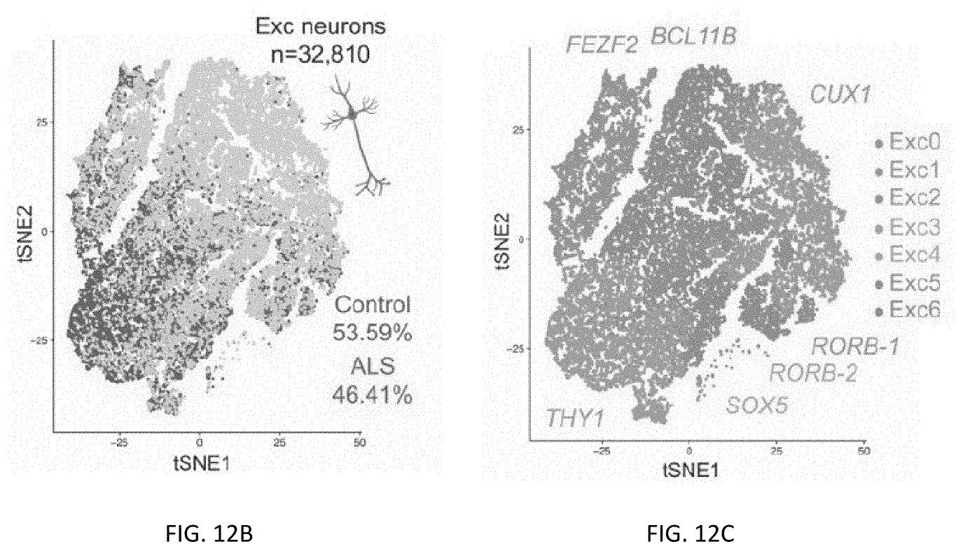

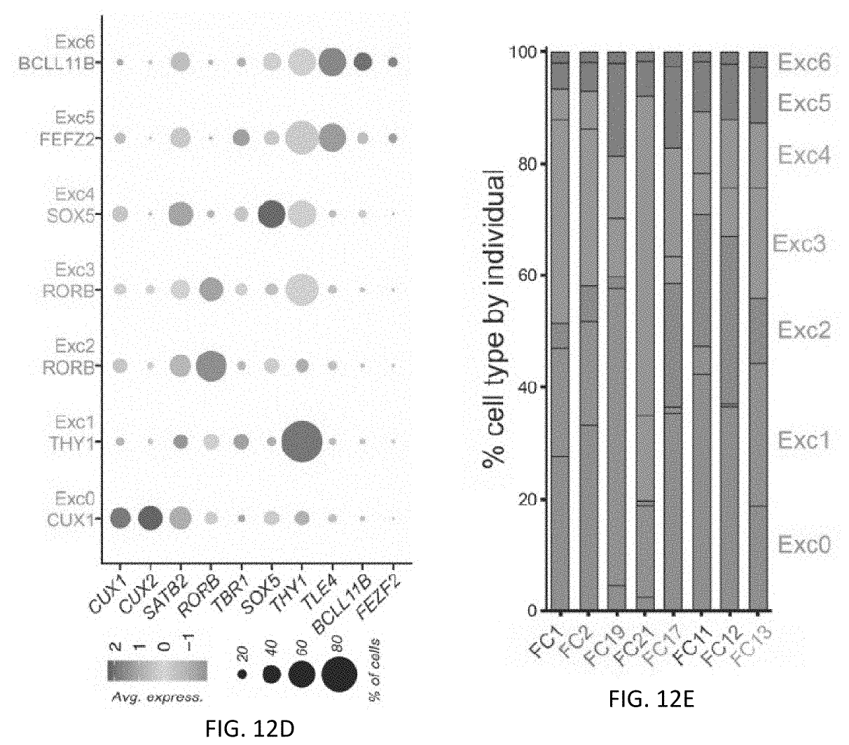

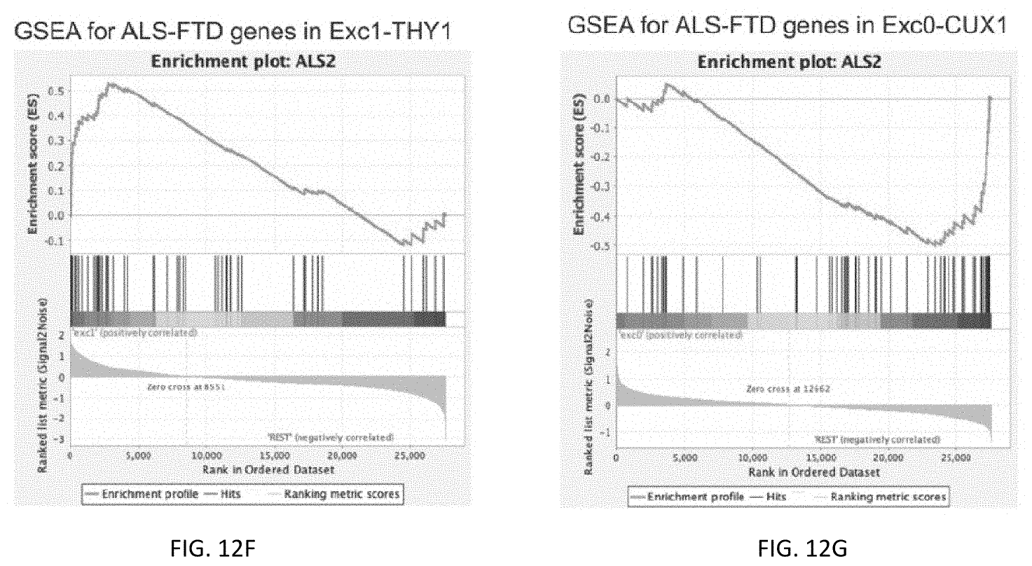

It was first asked whether analysis of expression patterns of ALS genetic risk factors (FIG. 12A) in the single nucleus dataset could provide insights into why certain cell types, including CSMNs, are more sensitive to degeneration. Initially a “module score” was computed for the expression of this set of risk genes in the different cell types defined above. To this end, a standardized z-score was generated for the expression of each risk gene, which was summed up as a total module score for the risk gene set and this score was normalized with transcript abundance from a randomly selected, comparable set of genes²⁰. Here, a positive score indicates higher expression of this risk gene set in a specific cell type compared to the average expression of the module across the collection of cell types in consideration. Also parallel module scores were computed for gene lists compiled from latest GWAS for neurological disorders that also affect the cortex: AD21,22 and MS23 75 (FIG. 6A). Interestingly, a clear enrichment for expression of ALS risk factors was not observed in any single broadly defined cell type (FIG. 6B). However, enriched expression of AD and MS genetic risk factors was found in microglia in the dataset, as predicted by previous studies²¹⁻²³ (FIGS. 6C-6D).

It was then considered whether combining the gene expression of all cortical excitatory neurons into a single profile might have prevented the identification of the enrichment of ALS risk gene expression in individual excitatory neuronal sub-types. To identify these excitatory neuronal subtypes, 32,810 likely excitatory neuron nuclei were examined by unbiased clustering and seven groups (Exc0-6) were identified that expressed known markers of different cortical layers equally distributed in the patient/control cohort (FIGS. 12B-12E). Analysis of the ALS genetic risk factors in these cells showed a positive score in THY1-expressing neurons, subgroup Exc1 and no other excitatory sub-type (Normalized Enrichment Score=1.834) (FIG. 6E, FIG. 12F). No excitatory neuronal sub-type specific enrichment for AD and MS risk gene modules was observed (FIGS. 6F-6G). THY1 is specifically enriched in human cortical layer 5¹³ and widely used as an expression marker for CSMNs^{13,24} Interestingly, neurons expressing upper layer marker CUX1 (Exc0) presented a lower-than-expected expression of these genes (NES=−1.730) (FIG. 12G). These findings were notable given the selective degeneration of CSMNs in ALS and findings from human samples²⁵ and mouse models²⁶ that suggest that superficial excitatory neuronal types have a lower propensity for pathologically accumulating TDP-43 relative to their deep layer counterparts.

### Distinct Alterations in Superficial and Deep-Layer Neurons

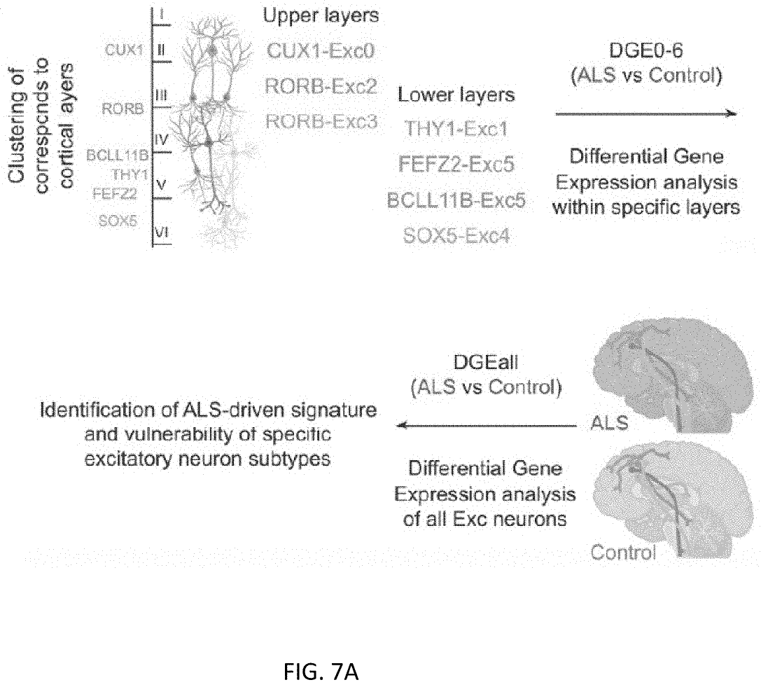

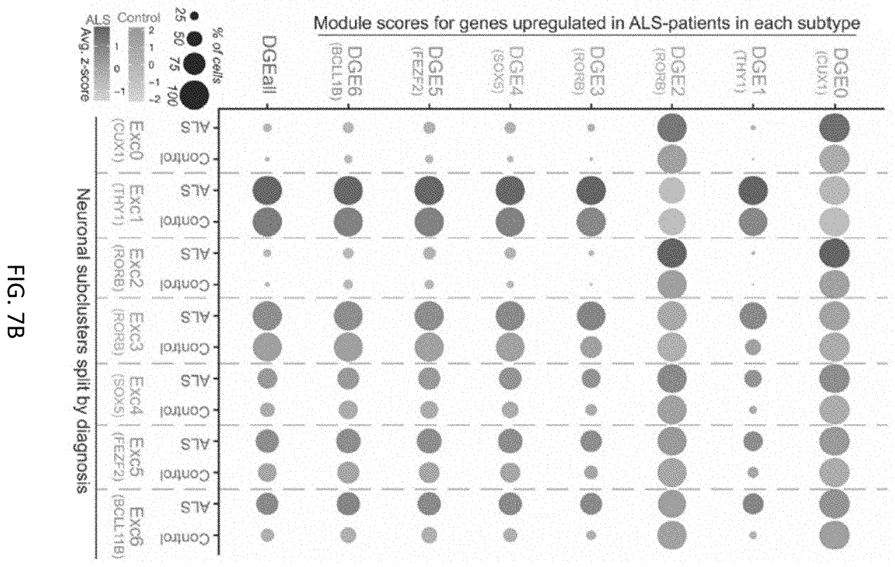

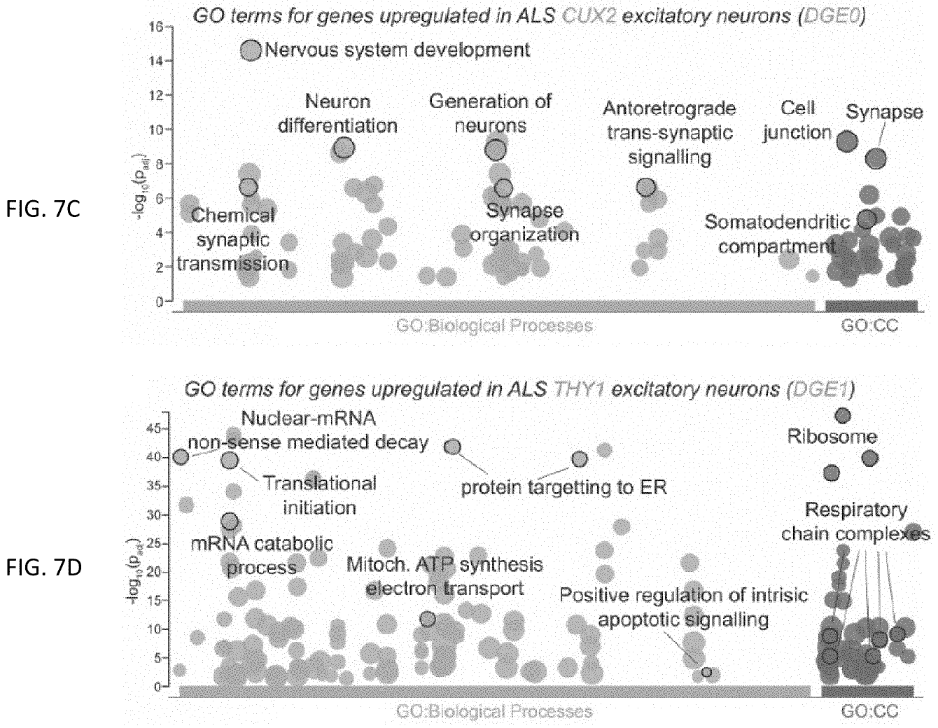

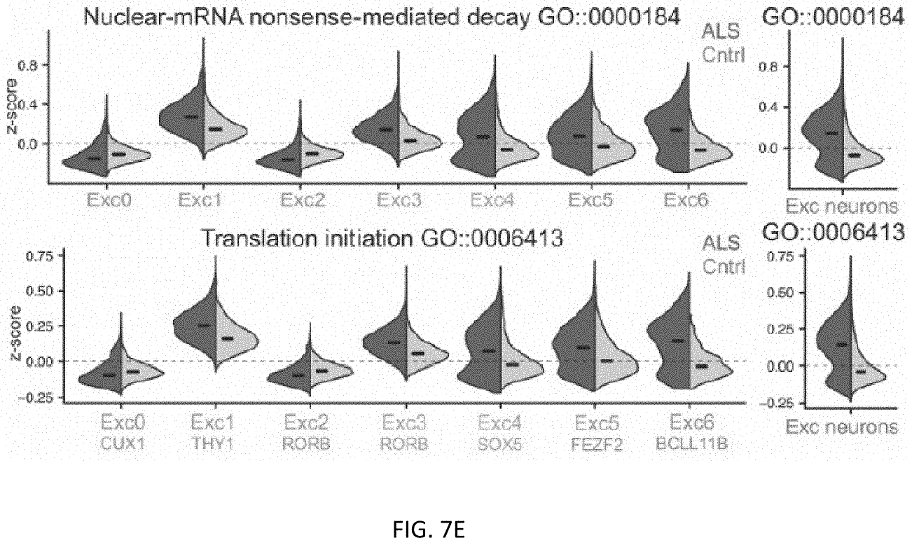

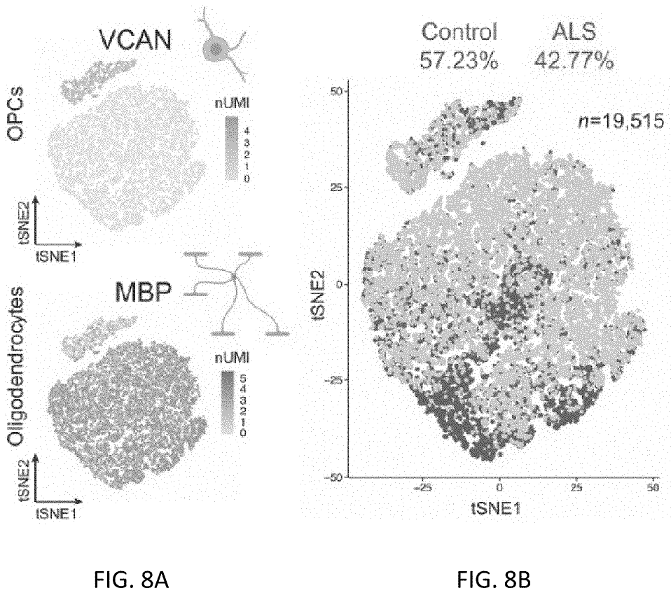

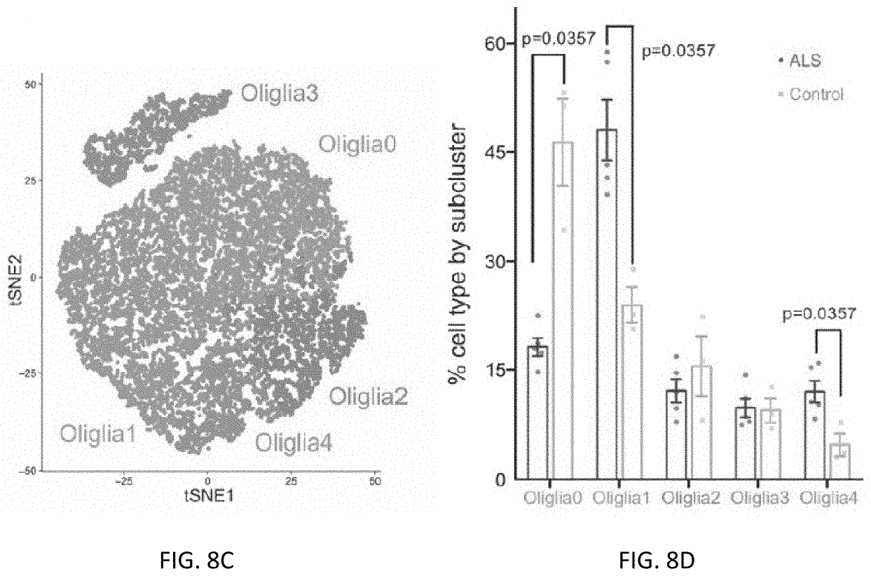

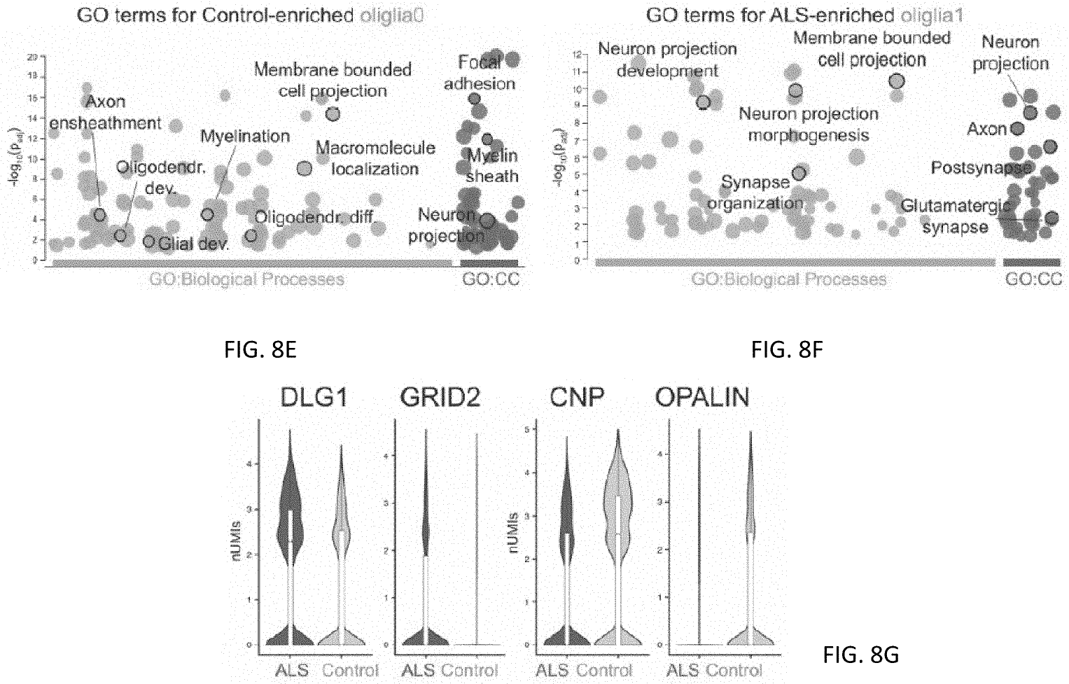

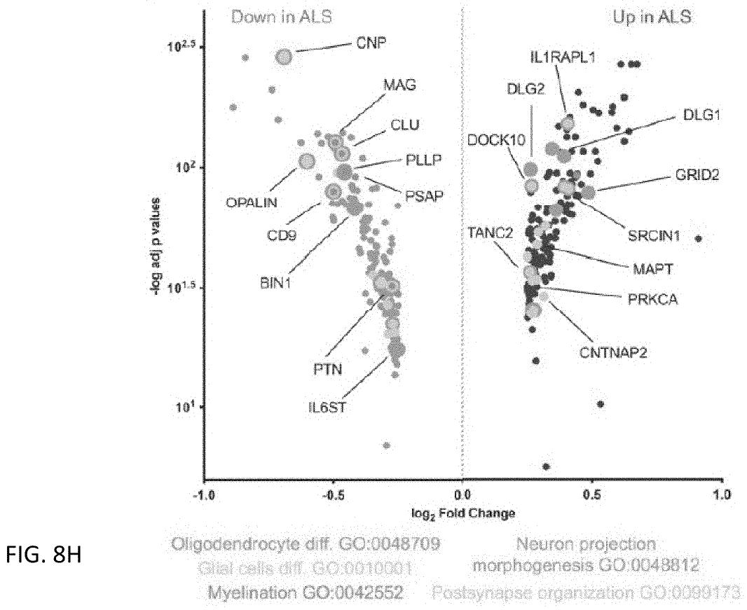

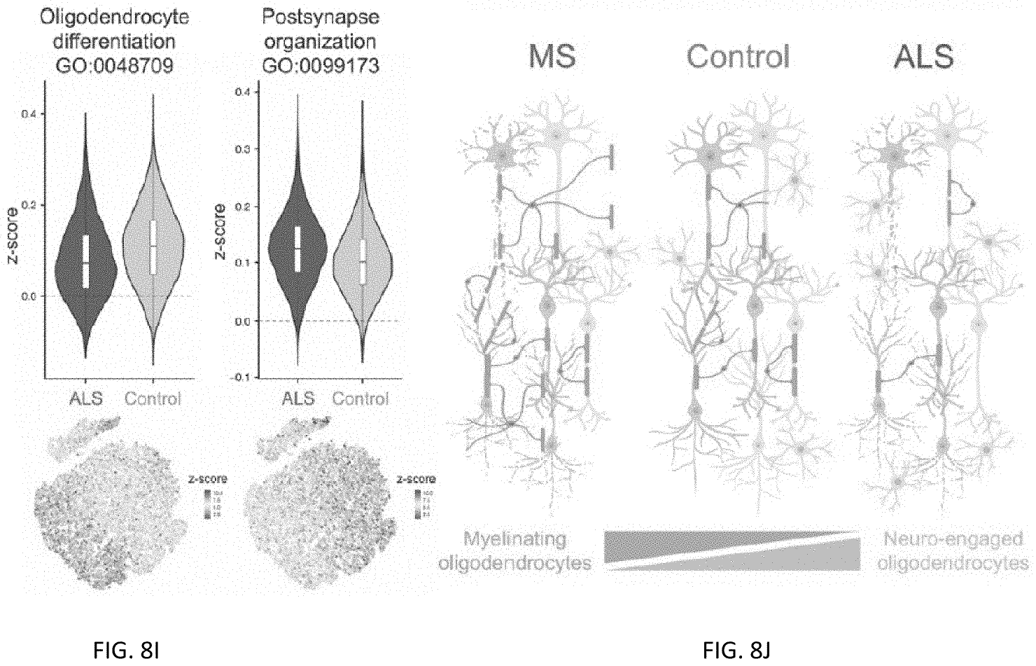

It was next examined how the enriched expression of ALS-FTD genes relates to changes that occur in excitatory neurons in response to ALS. Differential gene expression (DGE) analysis was conducted between neurons from patients and controls, across all excitatory neurons and within each excitatory subtype (FIG. 7A). To compare these signatures, genes significantly upregulated in patients globally (DGEall) and within each subgroup (DGE0-6) were selected, module-scores for each set were calculated and whether certain neuronal subtypes might have similar responses to ALS was investigated. This analysis showed a correlation between scores in groups expressing markers of lower layers (Exc1,4,5,6) and the global transcriptomic changes identified in patients (FIG. 7B), suggesting that pathological changes in the lower cortical layers are driving the observed alterations. For instance, groups expressing deep-layer CSMNs markers (THY1-Exc1, FEZF2-Exc5) shared many upregulated genes with each other and with the more global excitatory signature. Strikingly, genes upregulated in upper layers of the cortex (CUX1-Exc0), a region relatively spared of TDP-43 pathology, largely lacked these similarities (FIG. 8A).

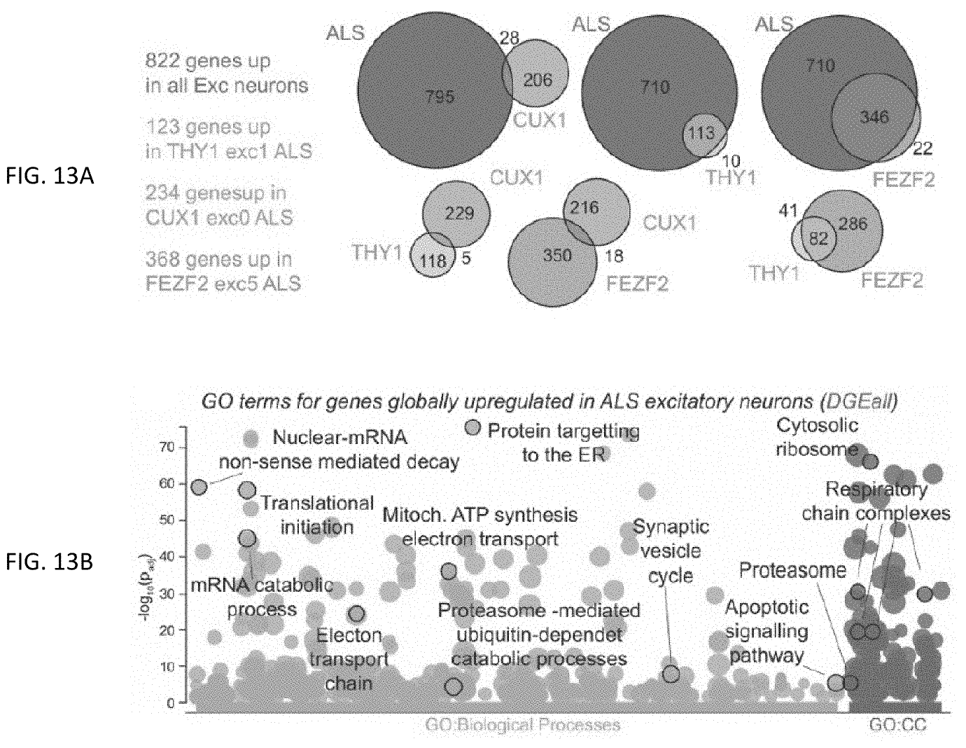

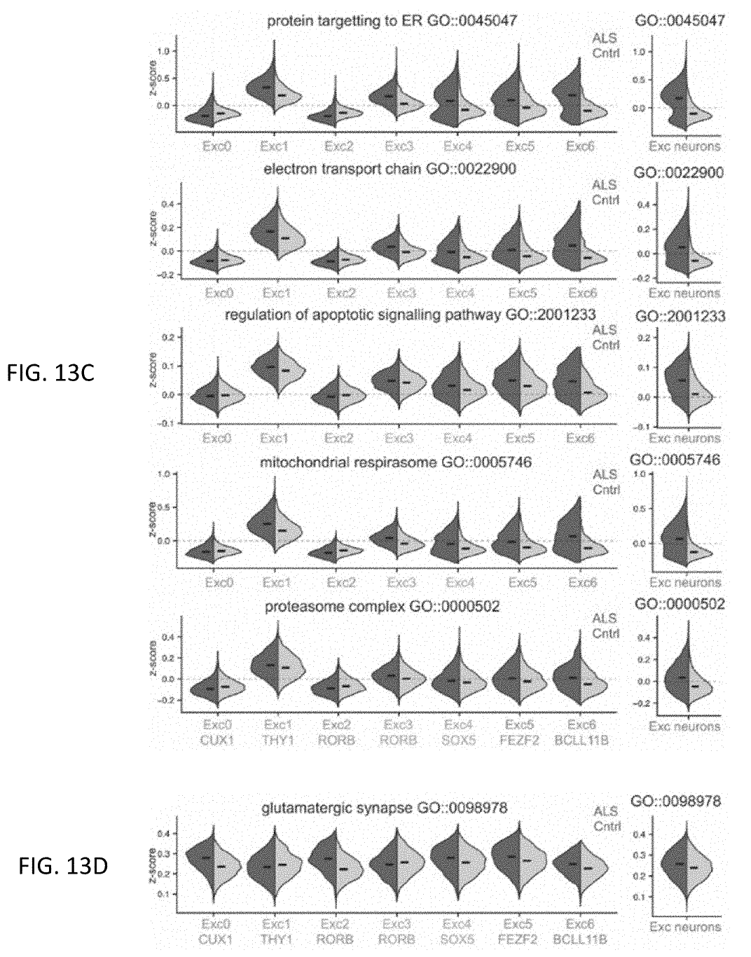

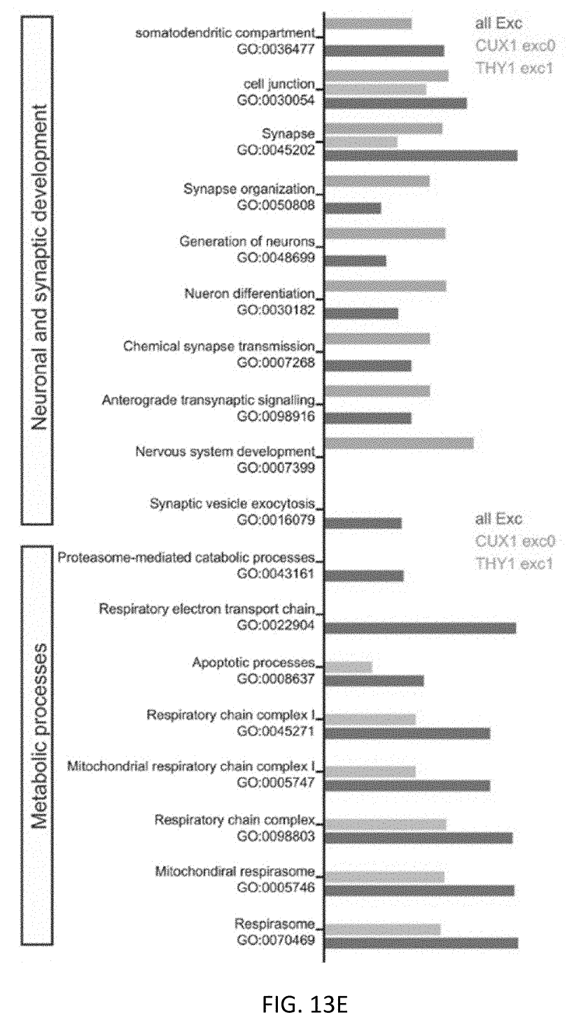

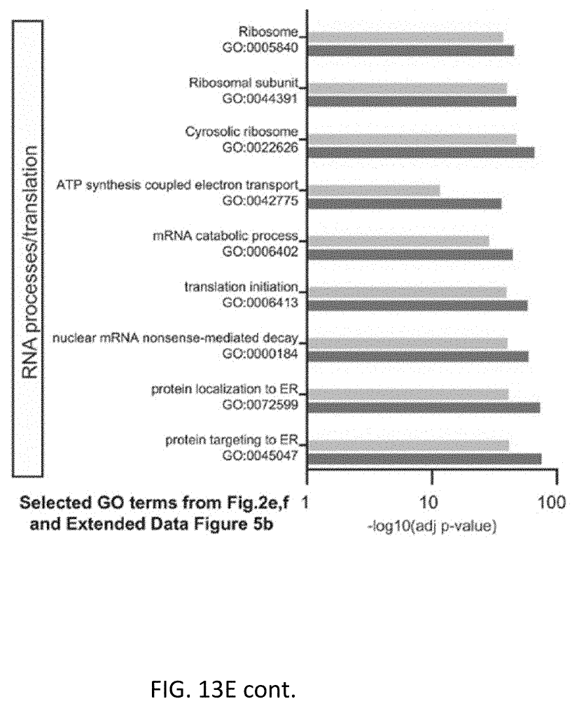

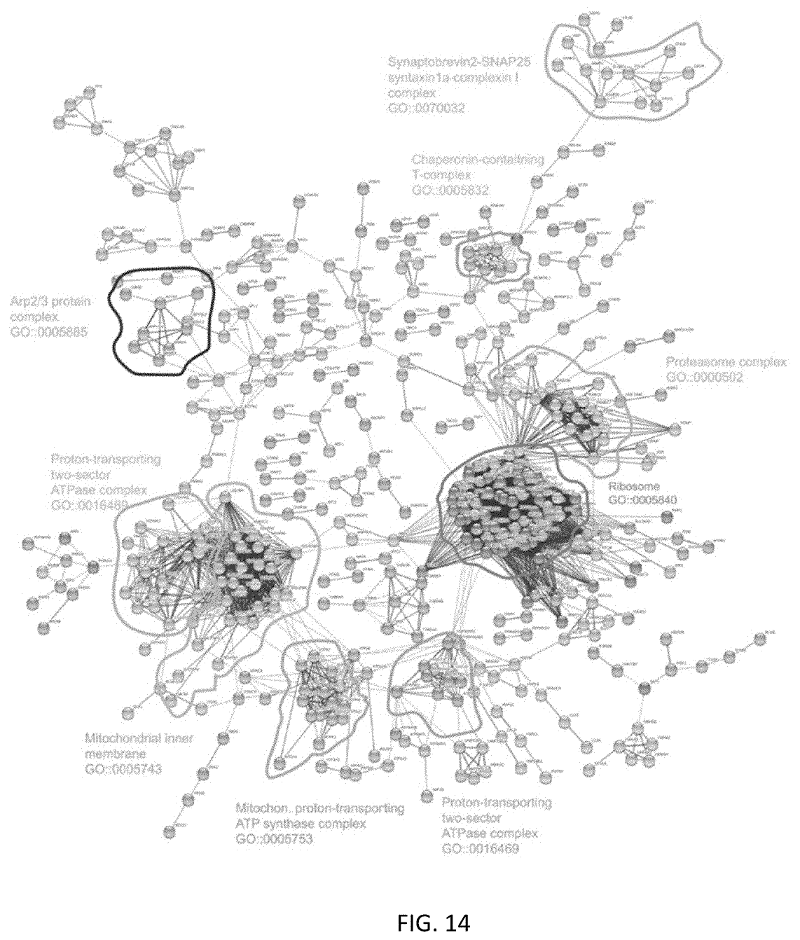

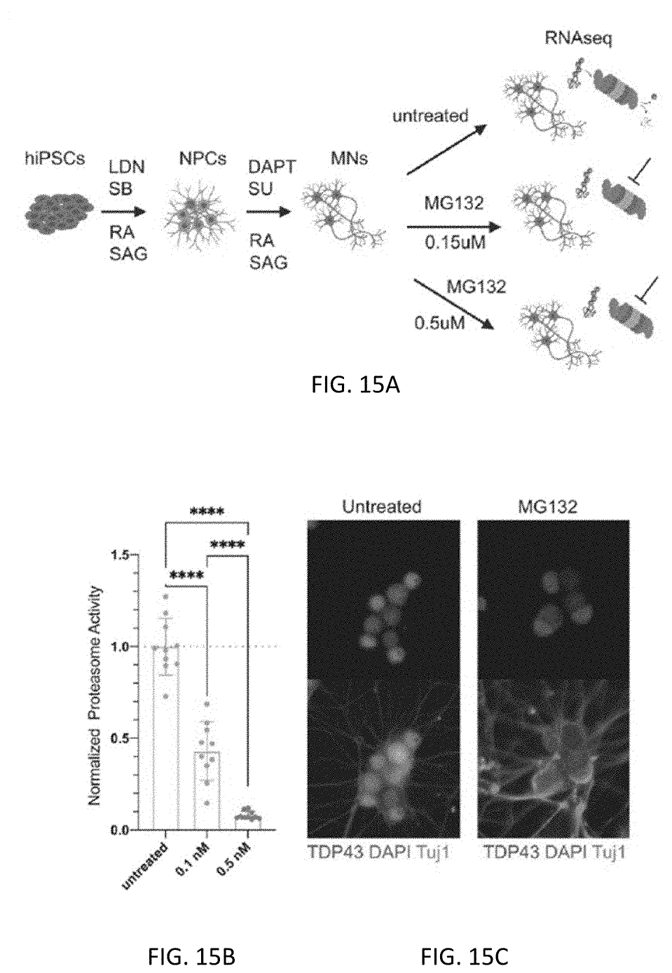

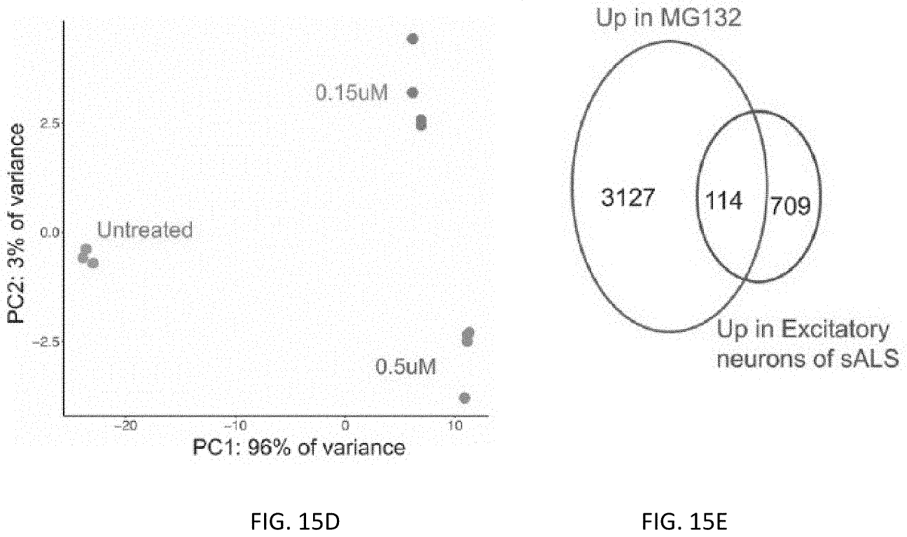

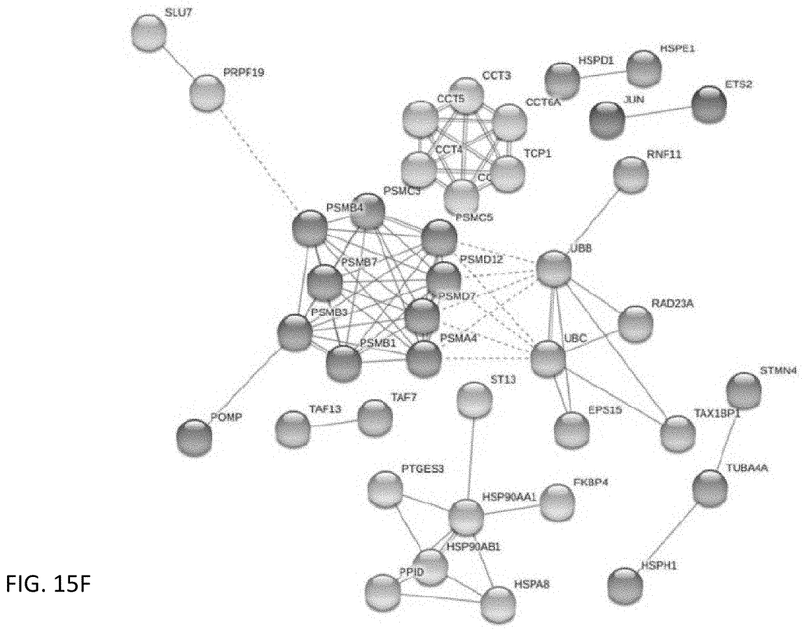

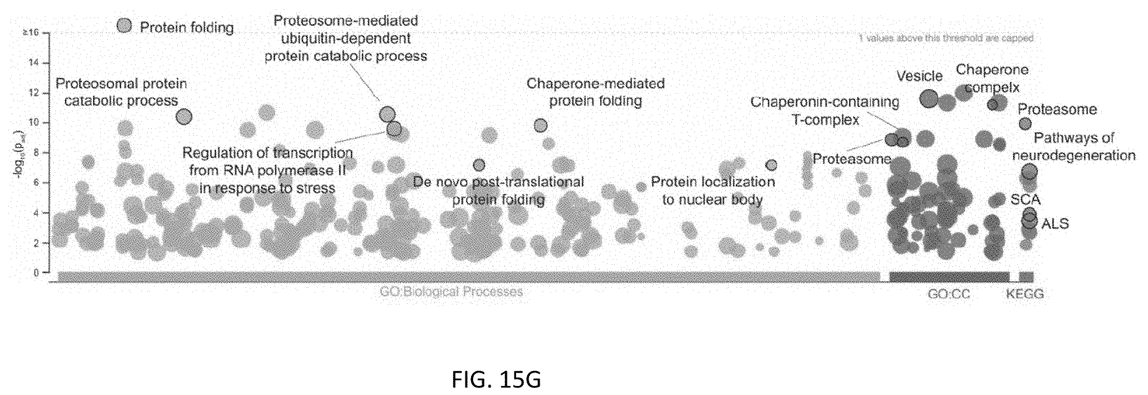

Subsequent Gene Ontology (GO) analysis showed that DEGs in CUX1-cells were associated with synaptic biology (FIG. 7C). In contrast, DEGs identified in THY1-cells were connected to cellular stresses previously associated with ALS^{1,2} (FIG. 7D) and many were shared with transcriptional changes identified in patients' excitatory cells as a whole (FIG. 8B). Combining differentially expressed genes with protein-protein interaction data suggested coordinated alterations in the expression of genes that function in ribosomal, mitochondrial, protein folding, and protein degradation pathways including the proteasome and the lysosome (FIG. 7E, FIG. 13C, FIG. 14). Interestingly, these pathways were specifically upregulated in neurons of deeper cortical layers rather than upper layer (FIG. 13E). It was next asked if aspects of these changes could be modeled in vitro using neurons derived from human Pluripotent Stem Cells (hPSC) (FIG. 15A). To recapitulate proteostatic stress MG132, a proteasome inhibitor, was applied to neurons²⁷ which was sufficient to induce nuclear loss of TDP-43, early hallmark of ALS (FIGS. 15B-15C). Subsequent RNA-sequencing of these neurons showed widespread transcriptomic changes after treatment, with many upregulated genes shared between stressed hPSC-neurons and neurons from sALS patients, especially proteasome subunits and heat-shock response-associated chaperonins (FIGS. 15D-15F). GO analysis of 114 shared alterations confirmed the upregulation of proteasome processes and chaperone complexes and suggests a connection to neurodegeneration in ALS (FIG. 15G). These findings show that proteasome inhibition can orchestrate alterations like those observed in deep layer neurons from ALS patients, underscoring those alterations in neuronal gene expression in ALS may in part be due to inhibition of proteostatic processes.

Oligodendroglial Respond to Neuronal Stress with a Neuronally-Engaged State

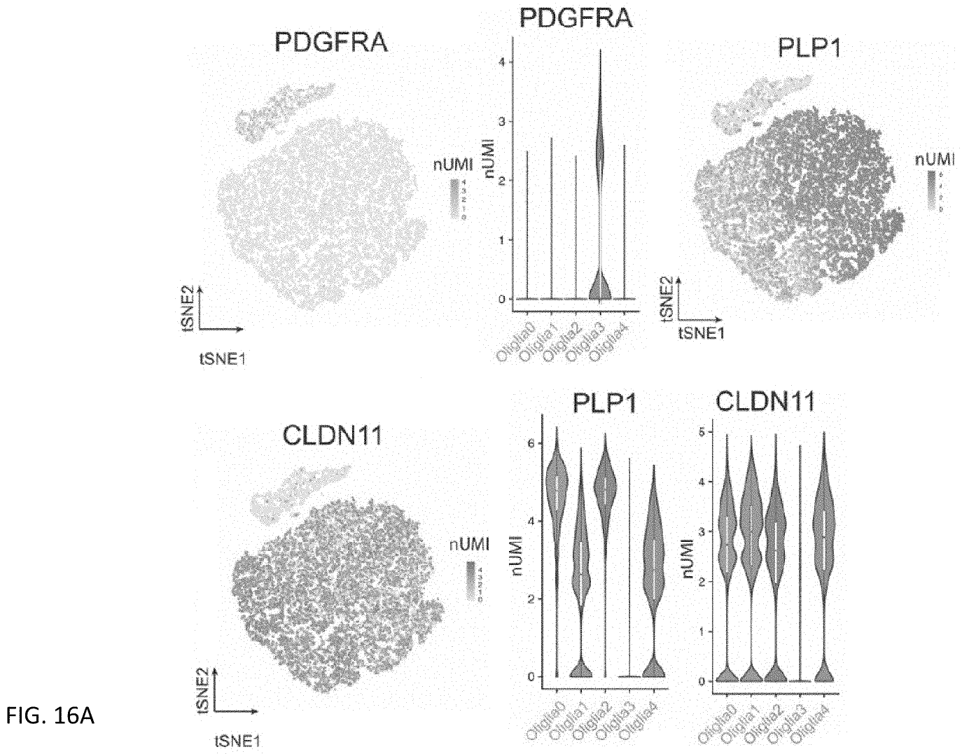

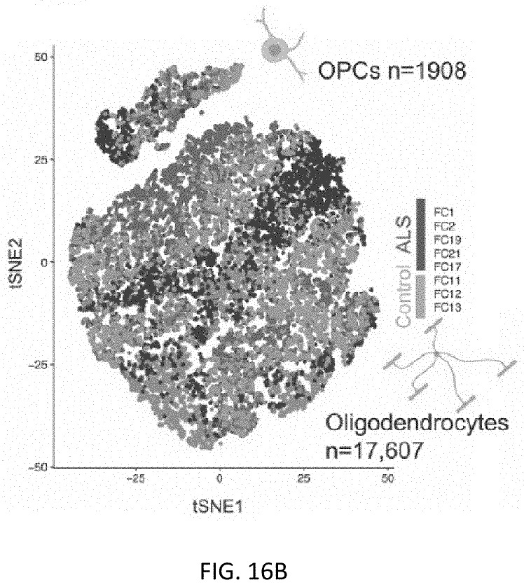

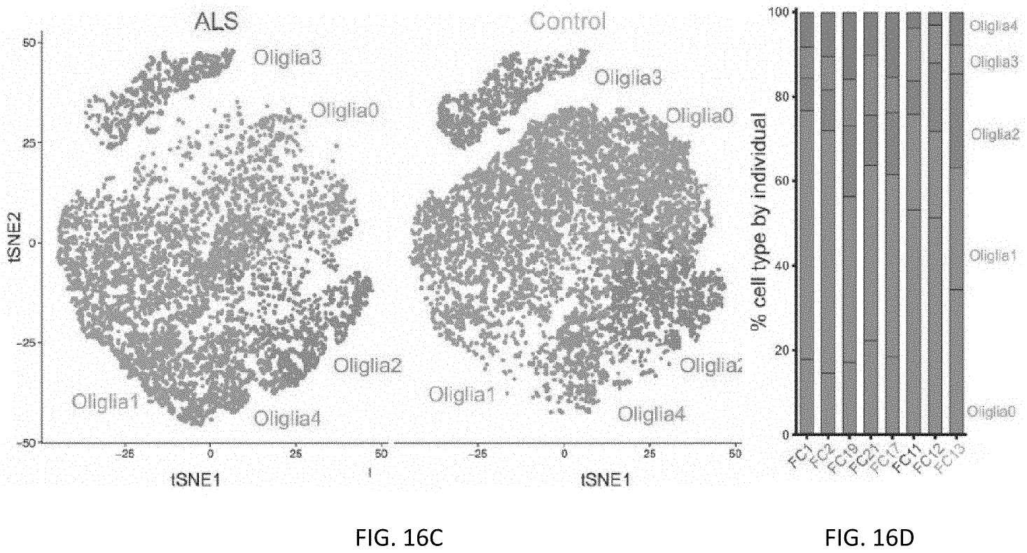

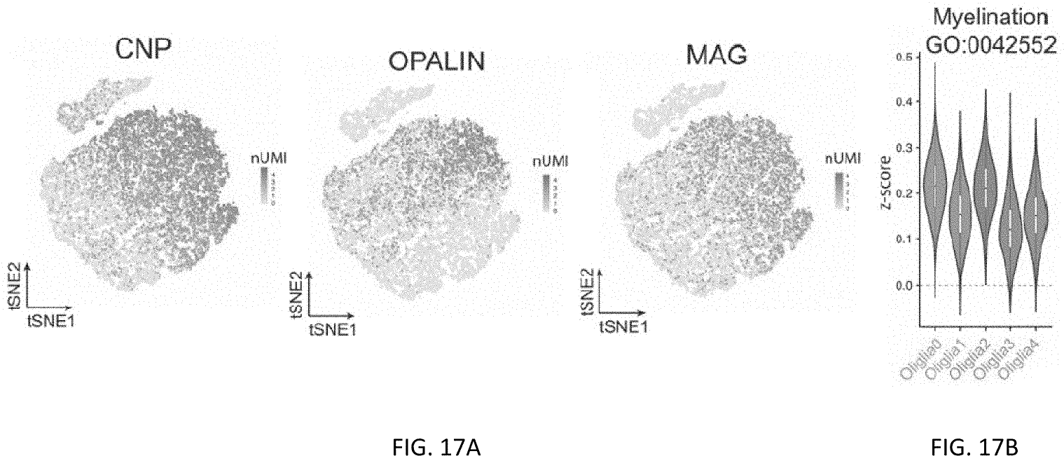

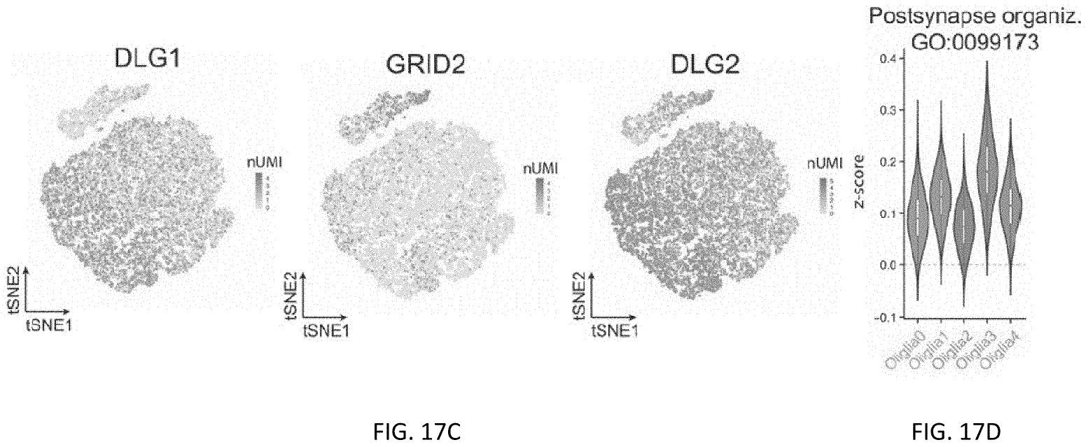

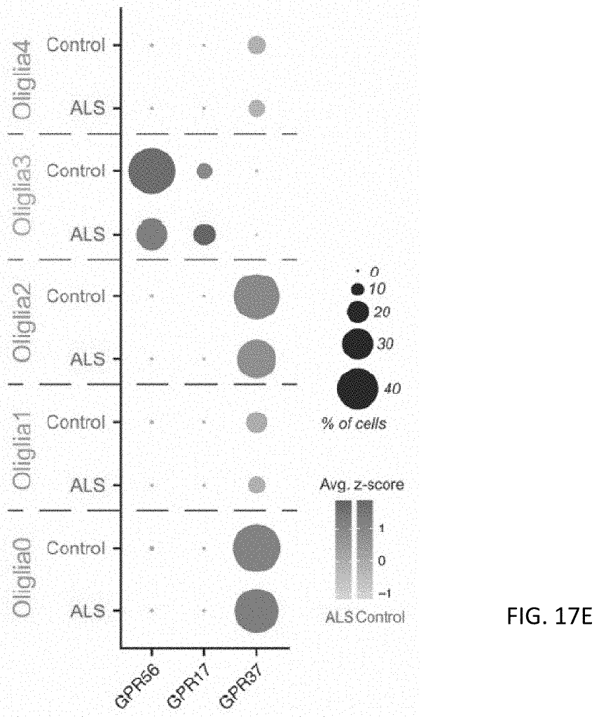

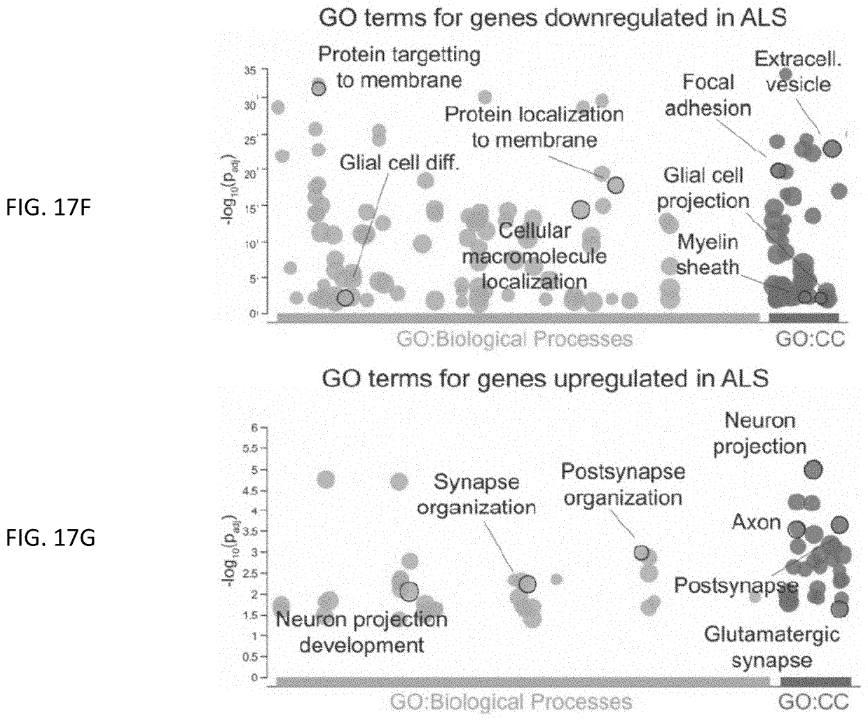

CSMNs are long-projection neurons that reach into the spinal cord and are dependent on robust axonal integrity²⁸, also changes in white matter and myelination have been associated with ALS patients¹¹. Nuclei from cells involved in myelination were therefore analyzed. The 19,151 nuclei from oligodendroglia were clustered in five groups: one of OPCs—Oliglia3, and four of oligodendrocytes—Oliglia0,1,2,4 (FIGS. 8A-8C, FIG. 16A). A significant depletion of ALS-nuclei in Oliglia0 was noted, whereas Oliglial and Oliglia4 were enriched in patients (FIG. 8D, FIGS. 16B-16D). GO analysis for genes enriched in each group compared to others, revealed that Control-enriched Oliglia0 was characterized by terms connected to oligodendrocyte development and myelination and expressed higher levels of myelinating genes, e.g. CNP, OPALIN, MAG (FIG. 8E, FIGS. 17A-17B). Conversely, ALS-enriched Oliglial show terms for neurite morphogenesis, synaptic organization and higher expression of postsynaptic genes DLG1, DLG2, GRID2 (FIG. 8F, FIGS. 17C-17D).

Global differential gene expression analysis supports a shift from a myelinating to a neuronally engaged state with upregulation of genes involved in synapse modulation and decrease of master regulators of myelination, as confirmed by GO analysis (FIGS. 8G-81, FIGS. 17F-171). Loss of myelination is exemplified by the expression of G-protein coupled receptors (GPRCs) that mark developmental milestones: GPR56, expressed in OPCs²⁹, and GPR37, expressed in myelinating cells³⁰, were lowly expressed in ALS-enriched subgroups and globally downregulated (FIG. 17E). Impaired myelination is consistent with previous studies identifying demyelination in sALS patients¹¹.

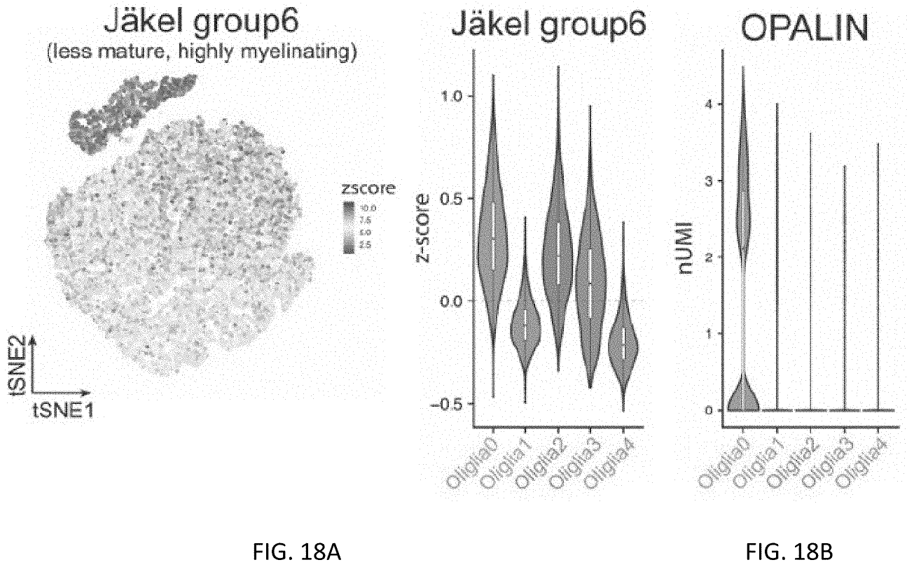

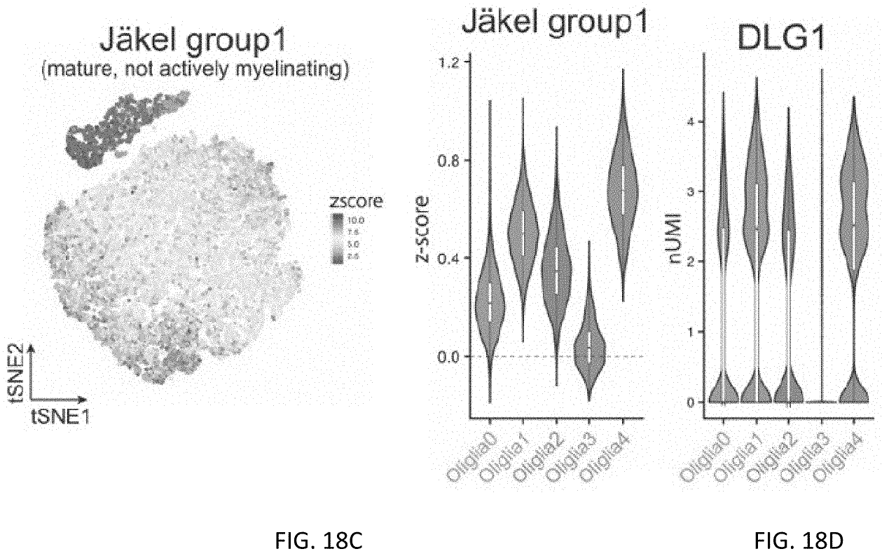

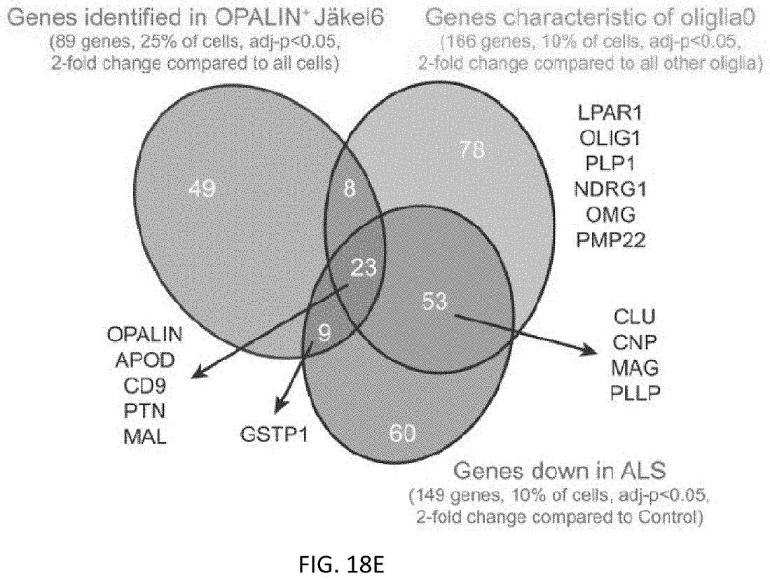

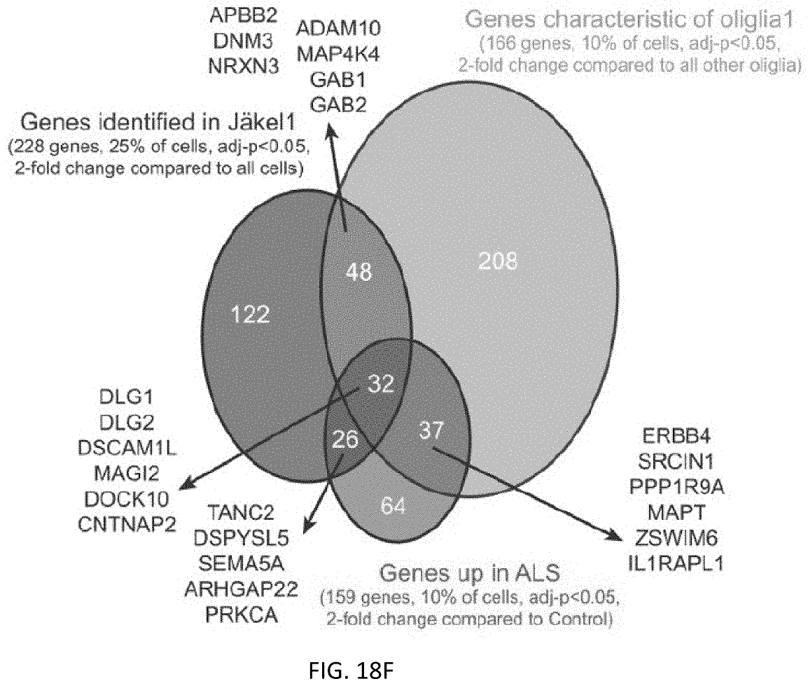

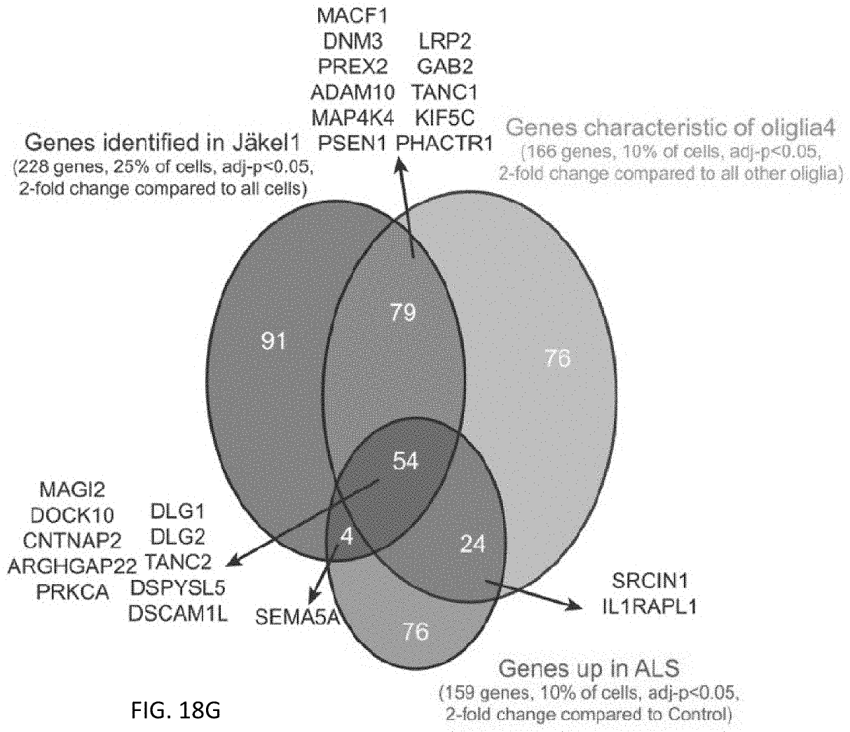

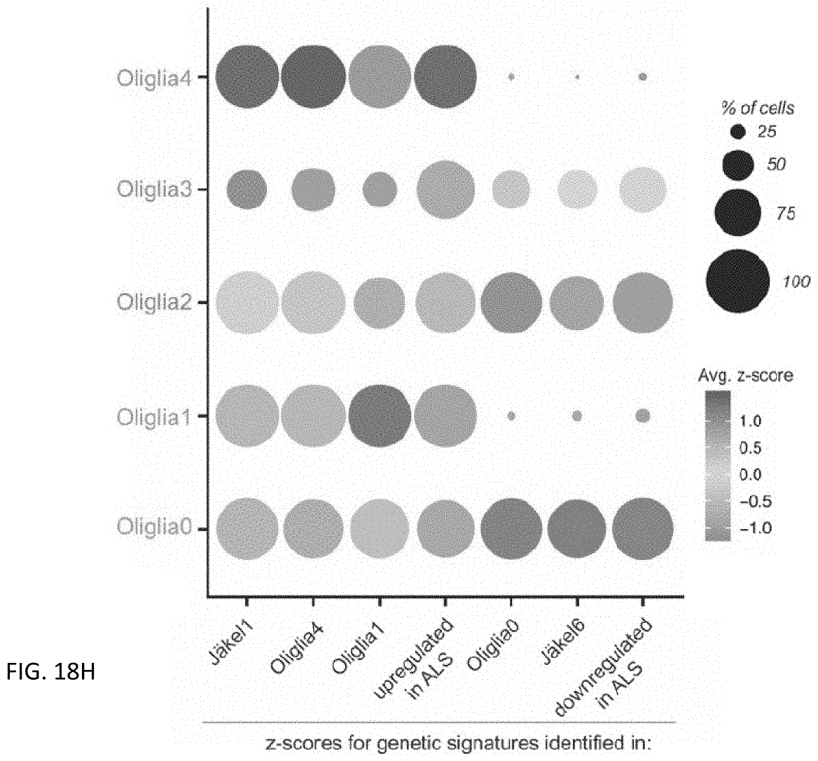

To explore the relevance of these changes, the study was compared with published reports that identified shifts in oligodendrocytes¹⁴. The correlation of gene modules from Jäkel et al.¹⁴ was investigated in this study, revealing that Control-enriched Oliglia0 most closely resembled highly myelinating, OPALIN⁺ cells from Jäkel (FIGS. 18A-18B), while ALS-enriched Oliglial and Oliglia4 aligned to not-actively myelinating Jäkel1 (FIGS. 18C-18D), with a high degree of shared genes (FIGS. 18E-18H). The data so far shows how activation of stress pathways in deep layer neurons is accompanied by a shift in oligodendrocytes from active myelination to oligo-to-neuron contact. This shift, that in MS is associated with replacement of myelin at lesions, has an opposite response in ALS, where we observed a more “neuro-supportive” state (FIG. 8J).

### Microglial Activation is Characterized by an ALS-Specific Endo-Lysosomal Response

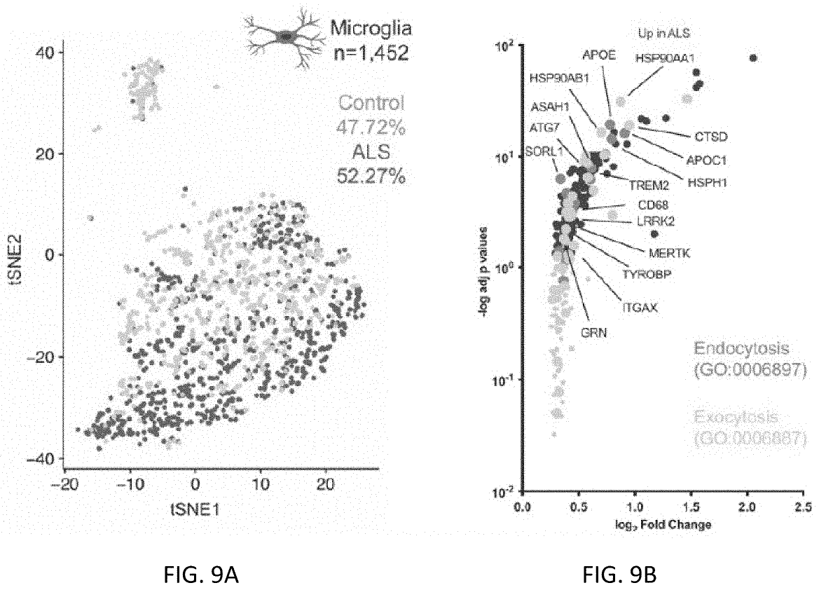

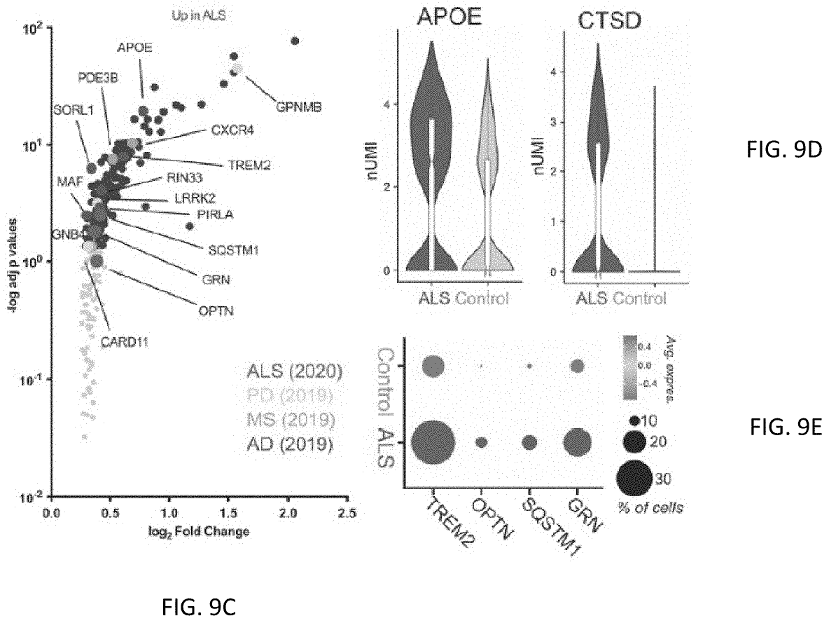

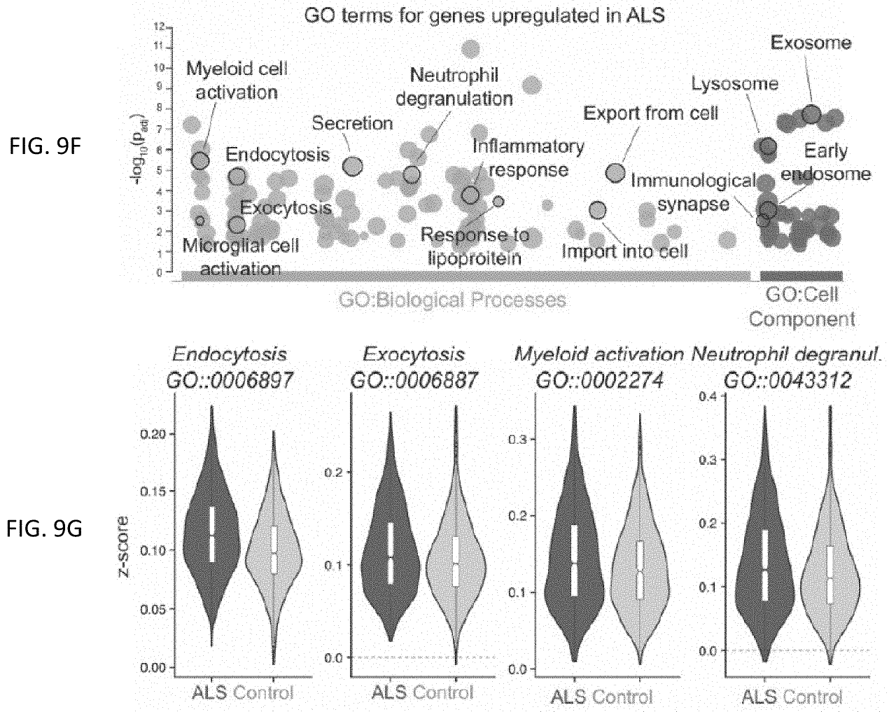

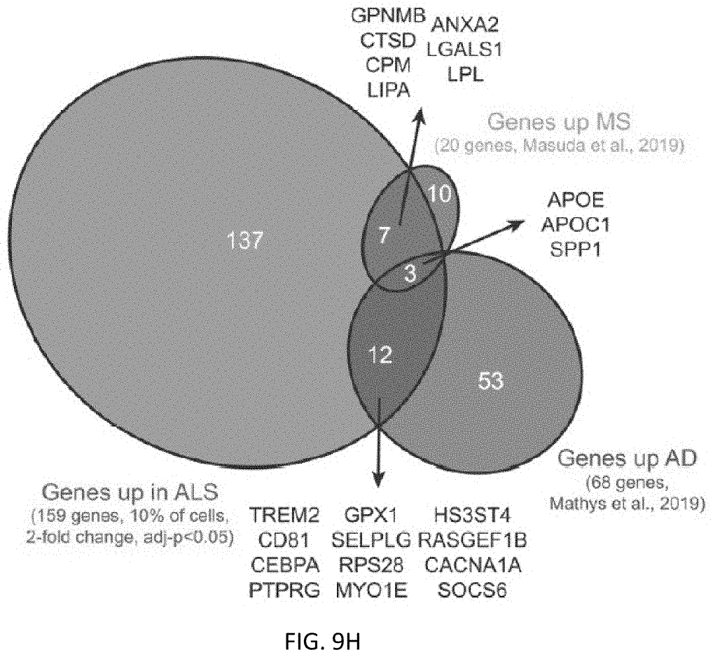

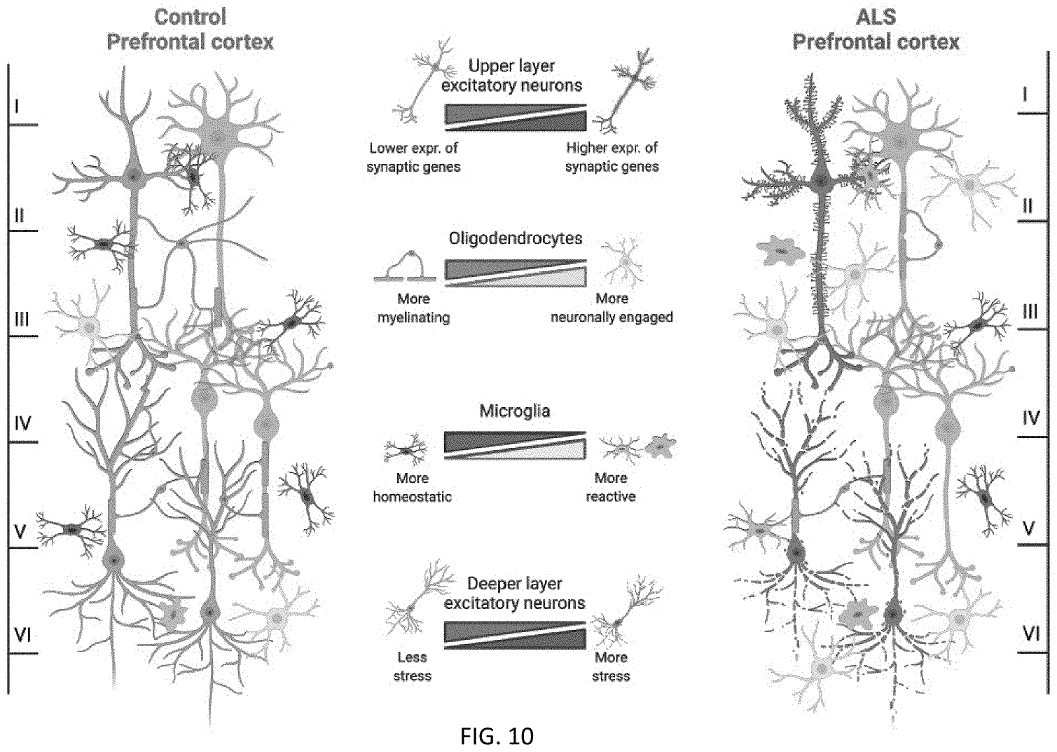

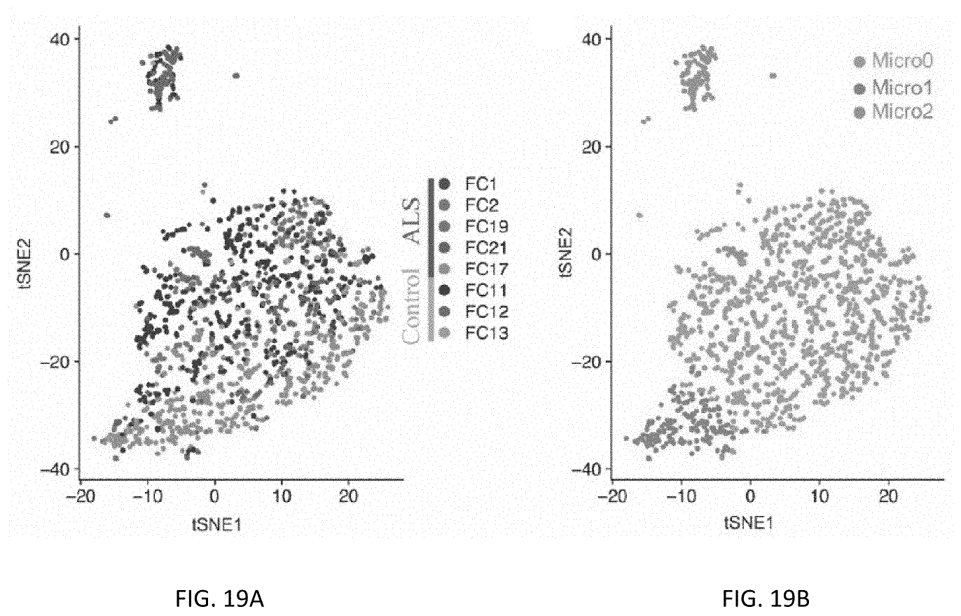

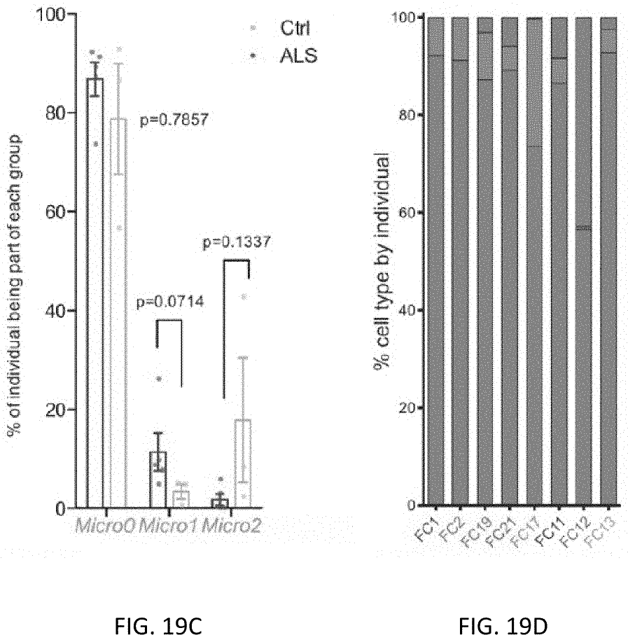

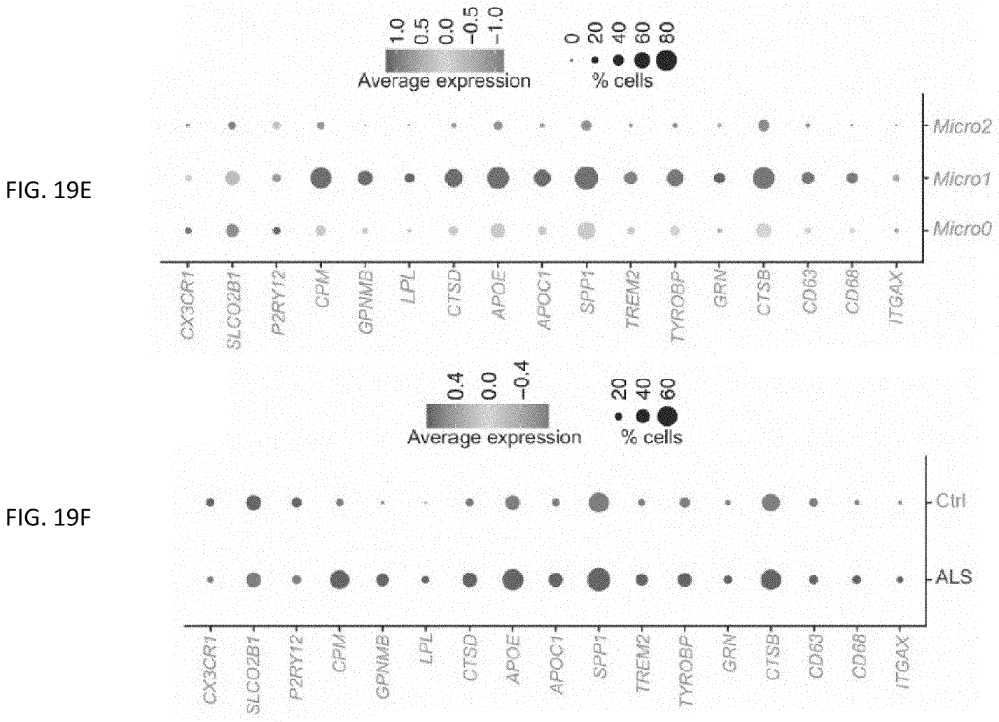

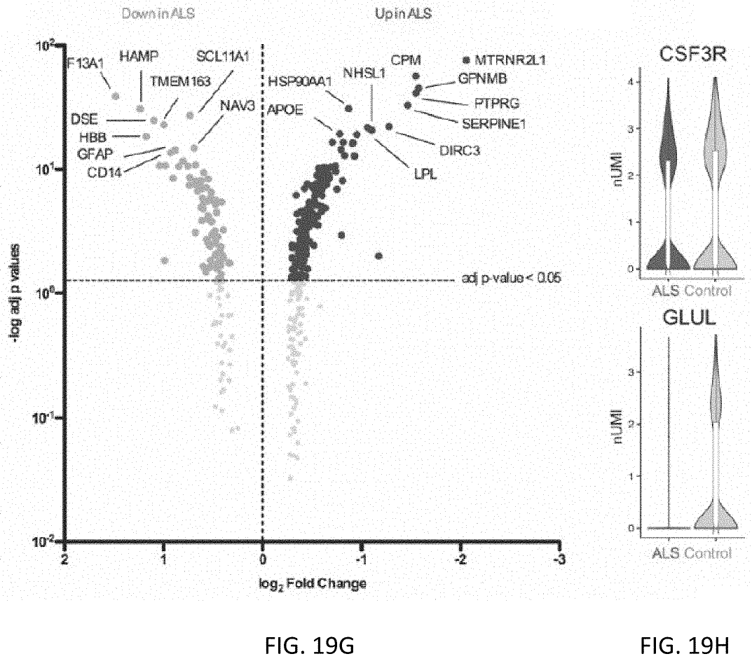

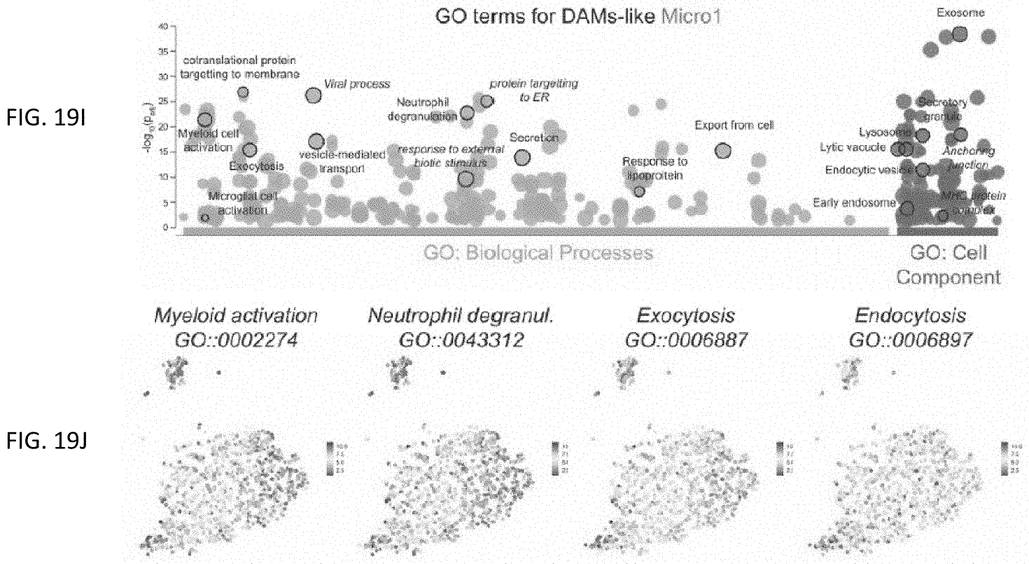

Mouse models³¹, patient samples⁶ and ALS-related genes function in myeloid cells³²⁻³⁴ have demonstrated the importance of microglia as modifiers of disease, so changes were interrogated in this cell type. In the 1,452 nuclei examined from microglia (FIG. 9A, FIG. 19A), 159 genes were identified as being upregulated in patients and, remarkably, with many being associated with endocytosis and exocytosis (e.g. TREM2, ASAH1, ATG7, SORL1, CD68). (FIG. 9B). Several of these genes were also associated with microglial activation (CTSD) and other neurodegenerative disorders (APOE) (FIGS. 9C-9D). Interestingly, several genes genetically associated with fALS were upregulated: OPTN, SQSTM1/p62, GRN (FIG. 9E). GO analysis for upregulated genes indicated activation of endo-lysosomal pathways, secretion and immune cells degranulation which have been previously proposed to occur in myeloid cells in ALS^{33,34} (FIGS. 9F-9G). Further subclustering identified three groups: homeostatic Micro0, “Disease Associated Microglia”-like Micro1, and cycling Micro2 (FIGS. 19B-19D). Notably, genes that characterized Micro1 were also upregulated in sALS (FIGS. 19E-19F), with downregulation of homeostatic genes and upregulation of reactive pathways (FIGS. 19G-19J).

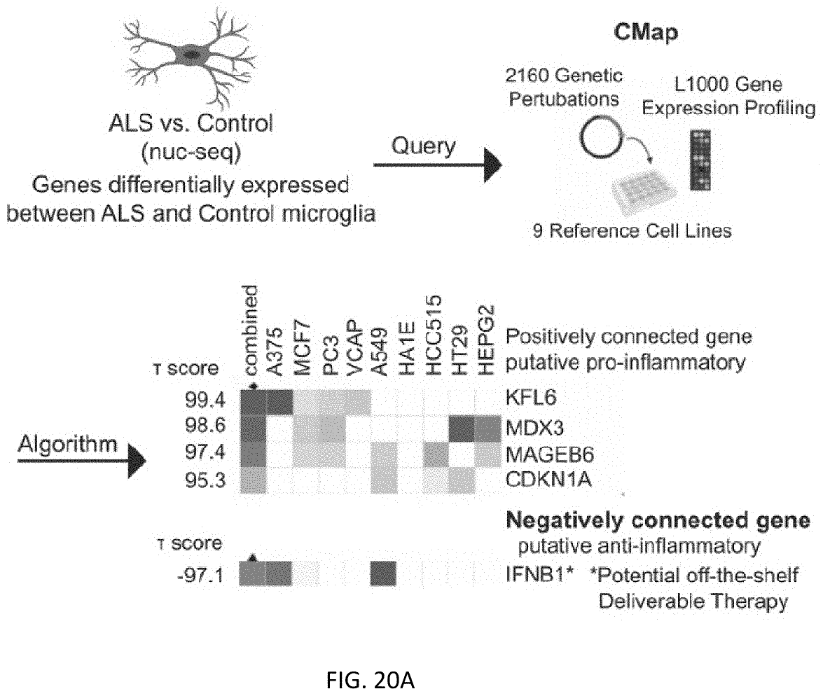

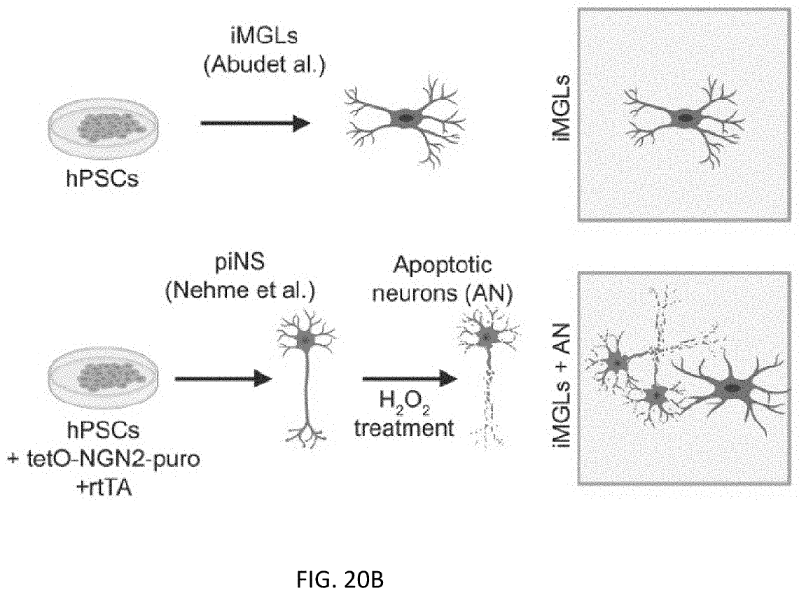

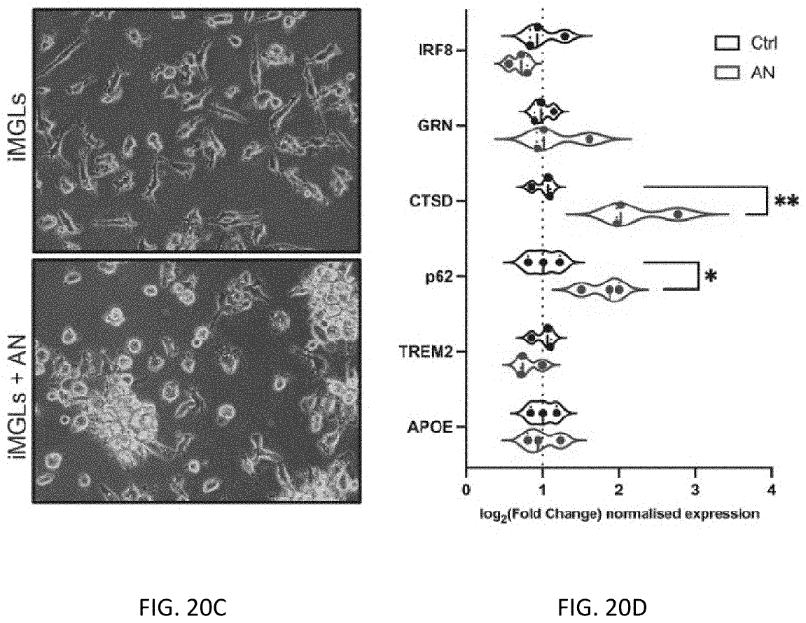

To identify modulators of this signature, the Connectivity Map (CMap) pipeline³⁵ was used, which contains gene expression data of 9 human cell lines treated with thousands of perturbations and allows association between a given transcriptomic signature and a specific perturbation. This analysis revealed that genes dysregulated in sALS microglia positively correlated with regulators of cell cycle and senescence, KLF6 and CDKN1A/p21, suggesting an exhaustion of microglial proliferation might be occurring in ALS. On the other hand, a negative correlation with a type I-interferon-associated response (IFNB1) was found, which is targeted in treatments for other neurological diseases to reduce inflammation³⁵ (FIG. 20A). Given the strong signature of homeostatic stress identified in deep layer neurons, it was considered whether changes seen in microglia might be caused by interactions with degenerating neurons. To test this idea, microglia-like cells (iMGLs)³⁶ and neurons (piNs)³⁷ were separately differentiated from hPSCs, triggered neuronal apoptosis and then introduced apoptotic neurons to iMGLs in culture (FIGS. 20B-20C). Quantitative assessment of representative transcripts by RT-qPCR confirmed that apoptotic neurons lead to the significant upregulation of genes involved in the endo-lysosomal trafficking pathways identified in microglia from ALS patients (FIG. 20D) suggesting that microglial changes are, at least in part, a response to degenerating neurons in sALS.

It was next asked whether the microglial changes that we found were a general response to neuronal disease or restricted to ALS. By comparing the results with published snRNA-seq studies on human microglia in AD¹⁵ and MS³⁸, it was identified that dysregulation of lipid metabolism (APOE, APOC1, SPP1) was a common feature, and that many genes associated with DAMs were shared between ALS and MS (GPNMB, CTSD, CPM, LPL) and ALS and AD (e.g. TREM2) (FIG. 9H). Genes specifically upregulated in ALS were related to vesicle trafficking, myeloid cell degranulation and the lysosome (e.g., SQSTM1, GRN, ASAH1, LRRK2, LGALS3). This evidence suggests the induction of a shared reactive state of microglia in neurodegenerative diseases through the TREM2/APOE axis. Yet in ALS neuronal death more specifically activates changes in transcripts connected to dysfunctional endo-lysosomal pathways.

### Discussion

A key question in the study of neurological disease is why certain neuronal types are more or less susceptible to degeneration in a particular condition. In this study, the enrichment for expression of ALS risk genes was identified in a class of CSMNs, which suggests clear mechanisms for their sensitivity to degeneration in ALS³⁹. First, the findings suggest that the higher expression of these risk factors renders CSMNs potentially more sensitive to gain-of-function mutant variants in ALS-associated genes than other neuronal sub-types. Secondly, it implies that these neurons may have a constitutively heightened need for expression of certain risk factor genes, which may be burned by rare heterozygous loss of function mutations or altered in expression by regulatory variants. Strikingly, this enrichment was not recapitulated for risk factors connected to AD and MS in the CSMNs data, it was instead replicated to be more enriched for expression in microglia.

Additionally, a broadly shared transcriptomic signature of induction of homeostatic stress pathways was identified in specific classes of deep layer excitatory neurons. These alterations in translation, proteostasis and mitochondrial function have previously been implicated in mouse models of ALS^{1,2}. This study indicates aligned changes occurring in deep-layer neuronal cell classes and highlights their cell-type specificity of these alterations. Importantly, human neuronal models were used to test whether a subset of these changes in gene expression were likely to be direct result of proteasome inhibition and this was found to be the case.

Emerging studies have shown that glial cells are important disease modulators in ALS. For instance, defects in oligodendrocyte maturation and myelination are present in SOD1-G93A mice and removing toxic SOD1 from this lineage improves survival¹¹. In this study, it was demonstrated that changes in expression of transcripts involved in oligodendrocyte differentiation, myelination and synapse organization occur in ALS and may therefore contribute to neuronal degeneration or alternatively may be a coordinated response to the disease. Additionally, the gene expression changes in this lineage in ALS appear to be in polar opposition to those described in MS¹⁴. Moreover, perturbations in key myelin-regulators were revealed, such as OPALIN, CNP, and MAG, across multiple oligodendrocyte clusters but in these cells only, as opposed to AD where myelination-related changes were present across multiple cell types¹⁵.

The role that synaptic apparatus and myelin assume in modulating neuronal excitability raises the question as to how regulation of synaptic signaling by oligodendrocytes might benefit neuronal survival. These changes are especially interesting if coupled with the finding concerning the upregulation of synaptic transcripts here identified in CUX1⁺ upper layer excitatory neurons and the documented loss of postsynaptic density molecules in CSMNs in ALS⁴⁰ and might relate to the changes in physiology observed in patients⁴¹. These observations suggest a response of the Cortico-Spinal motor circuit that attempts to compensate for the loss of neuronal inputs to the spinal cord and suggests that shifting oligodendroglial states may complement efforts aimed to alter excitatory inputs into CSMNs⁴¹.

Finally, distinct transcriptional perturbations were found in ALS-associated microglia, particularly in endo-lysosomal pathways. ALS-associated gene C9orf72 has been implicated in endosomal trafficking and secretion in myeloid cells³³′³⁴ and the upregulation of lysosomal constituents, e.g. CTSD, was identified in this study and confirmed by others in patients⁴². Coupled with the upregulation of fALS/FTD-associated genes SQSTM1/p62, OPTN, TREM2 and GRN, this suggests a mechanistic convergence on vesicle trafficking and pro-inflammatory pathways that may initiate and/or exacerbate the homeostatic-to-DAM transition in ALS. This observation underlines that the clear enrichment of ALS-related genes identified in CSMNs might not be the only genetic driver of the disease and could be coupled with processes engaging disease related genes in different cells, i.e. microglia. Changes in senescence and interferon-responsive genes were also delineated, as confirmed by others in C9orf72-ALS⁴³. Overall, differentially expressed transcripts in microglia had partial overlap with those in microglia surrounding amyloid plaques in AD^{15,16} and microglia associated with demyelinating lesions in MS³⁸, suggesting that partially shared but not altogether identical pathways are engaged in these neurodegenerative diseases, which clearly warrants further study.

In summary, it was shown that CSMNs harbour significantly higher expression of a collection of genetic risk factors for ALS/FTD that are also expressed in other deep-layer neuronal cell types but are depleted in their expression in excitatory neurons with more superficial identities. It was hypothesized that this intrinsically higher expression of disease-associated genes in putative CSMNs might be at the bottom of a “first over the line” mechanism leading to initial degeneration of this cell-type, followed by other “less-vulnerable” deep-layer neurons. Overall, the data suggests that these alterations in CSMNs and other deep layer cortical neurons may trigger a cascade of responses: superficial neurons upregulate synaptic genes potentially to supplement for lost inputs to the cord; oligodendroglia shift from a myelinating to a neuronally-engaged state; microglia activate a pro-inflammatory state in response to neuronal degeneration. Future investigations should consider how the individual alterations to distinct cell-types are ordered in disease processes and now that they are further elaborated, their relative importance in disease progression.

### Methods

### Human Donor Tissue

Post-mortem human cortical samples from ALS patients and age-matched controls were obtained at Massachusetts General Hospital using a Partners IRB approved protocol and stored at −80° C.

### Isolation of Nuclei

RNA quality of brain samples was assessed by running bulk nuclear RNA on an Agilent TapeStation for RIN scores. Extraction of nuclei from frozen samples was performed as previously described⁴⁴. Briefly, tissue was dissected and minced with a razor blade on ice and then placed in 4 ml ice-cold extraction buffer (Wash buffer (82 mM Na2SO4, 30 mM K2SO4, 5 mM MgCl2, 10 mM glucose, and 10 mM HEPES, pH adjusted to 7.4 with NaOH) containing 1% Triton X-100 and 5% Kollidon VA64). Tissue was homogenized with repeated pipetting, followed by passing the homogenized suspension twice through a 26½ gauge needle on a 3 ml syringe (pre-chilled), once through a 20 μm mesh filter, and once through a 5 μm filter using vacuum. The nuclei were then diluted in 50 ml ice-cold wash buffer, split across four 50 ml tubes, and centrifuged at 500×g for 10 minutes at 4° C. The supernatant was discarded, the nuclei pellet was resuspended in 1 ml cold wash buffer.

### 10× Loading and Library Preparation

Nuclei were DAPI-stained with Hoechst, loaded onto a hemacytometer, and counted using brightfield and fluorescence microscopy. The solution was diluted to −176 nuclei/ul before proceeding with Drop-seq as described in ref.15¹⁷. cDNA amplification was performed using around 6000 beads per reaction with 16 PCR cycles. The integrity of both the cDNA and fragmented libraries were assessed for quality control on the Agilent Bioanalyzer as in ref⁴⁵. Libraries were sequenced on a Nova-seq S2, with a 60 bp genomic read. Reads were aligned to the human genome assembly (hg19). Digital Gene Expression files were generated with the Zamboni Drop-seq analysis pipeline, designed by the McCarroll group⁴⁴.

### Filtering of Expression Matrices and Clustering of Single Nuclei

A single matrix for all samples was built by filtering any barcode with less than 400 genes and resulting in a matrix of 27,600 genes across 119,510 barcodes. This combined UMI matrix was used for downstream analysis using Seurat (v3.0.2)¹⁸. A Seurat object was created from this matrix by setting up a first filter of min.cells=20 per genes. After that, barcodes were further filtered by number of genes detected nFeature_RNA>600 and nFeature_RNA<6000. Distribution of genes and UMIs were used as parameters for filtering barcodes. The matrix was then processed via the Seurat pipeline: log-normalized by a factor of 10,000, followed by regressing out UMI counts (nCount_RNA), scaled for gene expression.

After quality filtering, 79,830 barcoes and 27,600 genes were used to compute SNN graphs and t-SNE projections using the first 10 statistically significant Principal Components. SNN-graphed t-SNE projection was used to determine minimum number of clusters obtain at resolution=0.2 (FindClusters). Broad cellular identities were assigned to groups on the basis of differentially expressed genes as calculated by Wilcoxon rank sum test in FindAllMarkers(min.pct=0.25, log fc.threshold=0.25). One subcluster with specifically high ratio of UMIs/genes was filtered out resulting in 79,169 barcodes grouped in 7 major cell types of the CNS: excitatory neurons, oligodendrocytes, inhibitory neurons, astrocytes, endothelial cells, microglia, oligodendrocyte progenitor cells (OPCs). Markers for specific cell types were identified in previously published human scRNAseq studies¹⁹.

Analysis of cellular subtypes were conducted by subsetting each group. Isolated barcodes were re-normalised and scaled and relevant PCs were used for re-clustering as a separate analysis. This newly scaled matrix was used for Differential Gene Expression analysis with parameters FindAllMarkers(min.pct=0.10, log fc.threshold=0.25) and subclustering for identification of subgroups. Gene scores for different cellular subclusters were computed in each re-normalized, re-scaled sub-matrix using the AddModule function in Seurat v3.0.2.

### Gene Ontology, Interactome and Gene Set Enrichment Analyses

For GO terms analysis, we selected statistically significant up-regulated or down-regulated genes identified in each subcluster as described before (adj p-values<0.05, LFC=2). These lists were fed in the gProfiler pipeline⁴⁶ with settings: use only annotated genes, g:SCS threshold of 0.05, GO cellular components and GO biological processes (26 May 2020-9 Dec. 2020), only statistically significant pathways are highlighted. For oligodendrocytes cells (FIG. 18) statistically significant up-regulated genes identified in each subcluster as described before (adj p-values<0.05, LFC=2) were used for synaptic specific Gene Ontology analysis using SynGO⁴⁷ (12 Jun. 2020). Interactome map was built using STRING⁴⁸ protein-protein interaction networks, all statistically significant upregulated genes were used, 810 were identified as interacting partners using “experiments” as interaction sources and a high confidence threshold (0.700), only interacting partners are shown in FIG. 16. Gene Set Enrichment Analysis was performed using GSEA software designed by UC San Diego and the Broad Institute (v4.0.3)⁴⁹. Briefly, gene expression matrices were generated in which for each subcluster each individual was a metacell, lists for disease-associated risk genes were compiled using available datasets (PubMed—ALSFTD) or recently published GWAS for AD^{21,22} and MS²³.

### Generation of Microglia-Like Cells

Microglial-like cells were differentiated as described in Abud et al.³⁶. Briefly, hPSCs were cultured in E8 medium (Stemcell technologies) on Matrigel (Corning), dissociated with Accutase (Stemcell technologies), centrifuged at 300×g for 5 minutes, resuspended in E8 medium with 10 μM Y-27632 ROCK Inhibitor, 2M cells are transferred to a low-attachment T25 flask in 4 ml of medium and left in suspension for 24 hours. The first 10 days of differentiation are carried out in iHPC medium: IMDM (50%, Stemcell technologies), F12 (50%, Stemcell technologies), ITSG-X 2% v/v (ThermoFisher), L-ascorbic acid 2-Phosphate (64 ug/ml, Sigma), monothioglycerol (400 mM, Sigma), PVA (10 mg/ml; Sigma), Glutamax (1×, Stemcell technologies), chemically-defined lipid concentrate (1×, Stemcell technologies), non-essential amino acids (NEAA, Stemcell technologies). After 24h (day0), cells are collected and differentiation is started in iHPC medium supplemented with FGF2 (Peprotech, 50 ng/ml), BMP4 (Peprotech, 50 ng/ml), Activin-A (Peprotech, 12.5 ng/ml), Y-27632 ROCK Inhibitor (1 μM) and LiCl (2 mM) and transferred in hypoxic incubator (20% 02, 5% CO₂, 37° C.). On day 2, medium is changed to iHPC medium plus FGF2 (Peprotech, 50 ng/ml) and VEGF (Peprotech, 50 ng/ml) and returned to hypoxic conditions. On day4, cells are resuspended in iHPC medium supplemented with FGF2 (Peprotech, 50 ng/ml), VEGF (Peprotech, 50 ng/ml), TPO (Peprotech, 50 ng/ml), SCF (Peprotech, 10 ng/ml), IL-6 (Peprotech, 50 ng/ml), and IL-3 (Peprotech, 10 ng/ml) and placed into a normoxic incubator (20% 02, 5% CO₂, 37° C.). Expansion of haematopoietic progenitors is continued by supplementing the flasks with 1 ml of iHPC medium with small molecules every two days. On day 10, cells are collected and filtered through a 40 μm filter. The single cell suspension is counted and plated at 500,00 cells/well of a 6 well plate coated with Matrigel (Corning) in Microglia differentiation medium: DMEM/F12 (Stemcell technologies), ITS-G 2% v/v (Thermo Fisher Scientific), B27 (2% v/v, Stemcell technologies), N2 (0.5% v/v, Stemcell technologies), monothioglycerol (200 mM, Sigma), Glutamax (1×, Stemcell technologies), NEAA (1×, Stemcell technologies), supplemented with M-CSF (25 ng/ml, Peprotech), IL-34 (100 ng/ml, Peprotech), and TGFb-1 (50 ng/ml, Peprotech). Induced Microglia-like cells (iMGLs) are kept in this medium for 20 days with change three times a week. On day 30, cells are collected and plated on poly-D-lysine/laminin coated dishes in Microglia differentiation medium supplemented with CD200 (100 ng/ml, Novoprotein) and CX3CL1 (100 ng/ml, PeproTech), M-CSF (25 ng/ml, PeproTech), IL-34 (100 ng/ml, PeproTech), and TGFb-1 (50 ng/ml, PeproTech) until day 40.

### Feeding of Apoptotic Neurons to Microglia-Like Cells

For feeding assays, neurons were generated from human iPSCs using an NGN2 overexpression system as described previously³⁷. Day30 hiPSC-neurons “piNs” were treated with 2 μM H₂O₂ for 24 hours to induce apoptosis. Apoptotic neurons were gently collected from the plate and the medium containing the apoptotic bodies was transferred into wells containing day40 iMGLs. After 24 hours, iMGLs subjected to apoptotic neurons and controls were collected for RNA extraction.

### RNA Extraction and RT-qPCR Analysis

RNA was extracted with the miRNeasy Mini Kit (Qiagen, 217004). cDNA was produced with iScript kit (BioRad) using 50 ng of RNA. RT-qPCR reactions were performed in triplicates using 20 ng of cDNA with SYBR Green (BioRad) and were run on a CFX96 Touch™ PCR Machine for 39 cycles at: 95° C. for 15s, 60° C. for 30s, 55° C. for 30s. List of primers can be found in Appendix.

Generation of hiPSC-Derived Neurons for Bulk RNA Sequencing

Human embryonic stem cells were cultured in mTESR (Stemcell technologies) on matrigel (Corning). Neurons were generated from HuES-3-Hb9:GFP based on the motor neuron differentiation protocol previously described²⁷. Upon completion of the differentiation protocol, cells were sorted via flow-cytometry based on GFP signal intensity to yield GFP-positive neurons that were plated on PDL/laminin-coated plates (Sigma, Life technologies). Neurons were maintained in Neurobasal medium (Life Technologies) supplemented with N2 (Stemcell technologies), B27 (Life technologies), glutamax (Life technologies), non-essential amino acids (Life technologies), and neurotrophic factors (BDNF, GDNF, CNTF), and were grown for 28 days before the application of the proteasome inhibitors MG132 for 24 hrs.

RNA was extracted using RNeasy Plus kit (Qiagen), libraries were prepared using the Illumina TruSeq RNA kit v2 according to the manufacturer's directions, and sequenced at the Broad Institute core with samples randomly assigned between two flow chambers. The total population RNA-seq FASTQ data was aligned against ENSEMBL human reference genome (build GRCh37/hg19) using STAR (v.2.4.0). Cufflinks (v.2.2.1) was used to derive normalized gene expression in fragments per kilo base per million (FPKM). The read counts were obtained from the aligned BAM-files in R using Rsubread. Differential gene expression was analyzed from the read counts in DESeq2 using a Wald's test for the treatment dosage and controlling for the sequencing flow cell.

### Western Blot Analysis

For WB analyses, cells were lysed in RIPA buffer with protease inhibitors (Roche). After protein quantification by BCA assay (ThermoFisher), ten micrograms of proteins were preheated in Laemmli's buffer (BioRad), loaded in 4-20% mini-PROTEAN® TGX™ precast protein gels (BioRad) and gels were transferred to a PDVF membrane. Membranes were blocked in Odyssey Blocking Buffer (Li-Cor) and incubated overnight at 4° C. with primary antibodies. After washing with TBS-T, membranes were incubated with IRDye® secondary antibodies (Li-Cor) for one hour and imaged with Odyssey® CLx imaging system (Li-Cor). List of primary antibodies can be found in Appendix.

### Proteasome Activity Assay

Neurons were sorted in 96-wells plates and, after two weeks of maturation, treated for 24 hours. Cells were washed with 1×PBS, exposed to ProteasomeGlo® (Promega, G8660) and incubated for 30 minutes at RT. Fluorescence was measured using a Cytation™3 reader (BioTek).

### REFERENCES

1. Taylor, J. P., Brown, R. H., Jr., & Cleveland, D. W. Decoding ALS: from genes to mechanism. Nature 539, 197-206, doi:10.1038/nature20413 (2016).
2. Brown, R. H. & Al-Chalabi, A. Amyotrophic Lateral Sclerosis. N Engl J Med 377, 162-172, doi:10.1056/NEJMra1603471 (2017).
3. Wainger, B. J. & Lagier-Tourenne, C. Taking on the Elephant in the Tissue Culture Room: iPSC Modeling for Sporadic ALS. Cell Stem Cell 23, 466-467, doi: 10.1016/j.stem.2018.09.015 (2018).
4. Prudencio, M. et al. Distinct brain transcriptome profiles in C9orf72-associated and sporadic ALS. Nat Neurosci 18, 1175-1182, doi:10.1038/nn.4065 (2015).
5. Mordes, D. A. et al. Dipeptide repeat proteins activate a heat shock response found in C9ORF72-ALS/FTLD patients. Acta Neuropathol Commun 6, 55, doi:10.1186/s40478-018-0555-8 (2018).
6. D'Erchia, A. M. et al. Massive transcriptome sequencing of human spinal cord tissues provides new insights into motor neuron degeneration in ALS. Sci Rep 7, 10046, doi:10.1038/s41598-017-10488-7 (2017).
7. Tam, 0. H. et al. Postmortem Cortex Samples Identify Distinct Molecular Subtypes of ALS: Retrotransposon Activation, Oxidative Stress, and Activated Glia. Cell Rep 29, 1164-1177 e1165, doi:10.1016/j.celrep.2019.09.066 (2019).
8. Neumann, M. et al. Ubiquitinated TDP-43 in frontotemporal lobar degeneration and amyotrophic lateral sclerosis. Science 314, 130-133, doi:10.1126/science.1134108 (2006).
9. Suzuki, N. et al. The mouse C9ORF72 ortholog is enriched in neurons known to degenerate in ALS and FTD. Nat Neurosci 16, 1725-1727, doi:10.1038/nn.3566 (2013).
10. Ransohoff, R. M. How neuroinflammation contributes to neurodegeneration. Science 353, 777-783, doi: 10.1126/science.aag2590 (2016).
11. Kang, S. H. et al. Degeneration and impaired regeneration of gray matter oligodendrocytes in amyotrophic lateral sclerosis. Nat Neurosci 16, 571-579, doi:10.1038/nn.3357 (2013).
12. Boillee, S. et al. Onset and progression in inherited ALS determined by motor neurons and microglia. Science 312, 1389-1392, doi:10.1126/science.1123511 (2006).Abstract/FREE Full TextGoogle Scholar
13. Schirmer, L. et al. Neuronal vulnerability and multilineage diversity in multiple sclerosis. Nature 573, 75-82, doi:10.1038/s41586-019-1404-z (2019).
14. Jakel, S. et al. Altered human oligodendrocyte heterogeneity in multiple sclerosis. Nature 566, 543-547, doi:10.1038/s41586-019-0903-2 (2019).
15. Mathys, H. et al. Single-cell transcriptomic analysis of Alzheimer's disease. Nature 570, 332-337, doi:10.1038/s41586-019-1195-2 (2019).
16. Zhou, Y. et al. Human and mouse single-nucleus transcriptomics reveal TREM2-dependent and TREM2-independent cellular responses in Alzheimer's disease. Nat Med 26, 131-142, doi:10.1038/s41591-019-0695-9 (2020).
17. Macosko, E. Z. et al. Highly Parallel Genome-wide Expression Profiling of Individual Cells Using Nanoliter Droplets. Cell 161, 1202-1214, doi:10.1016/j.cell.2015.05.002 (2015).
18. Stuart, T. et al. Comprehensive Integration of Single-Cell Data. Cell 177, 1888-1902 e1821, doi:10.1016/j.cell.2019.05.031 (2019).
19. Lake, B. B. et al. Integrative single-cell analysis of transcriptional and epigenetic states in the human adult brain. Nat Biotechnol 36, 70-80, doi:10.1038/nbt.4038 (2018).
20. Tirosh, I. et al. Dissecting the multicellular ecosystem of metastatic melanoma by single-cell RNA-seq. Science 352, 189-196, doi:10.1126/science.aad0501 (2016).
21. Kunkle, B. W. et al. Genetic meta-analysis of diagnosed Alzheimer's disease identifies new risk loci and implicates Abeta, tau, immunity and lipid processing. Nat Genet 51, 414-430, doi: 10.1038/s41588-019-0358-2 (2019).
22. Jansen, I. E. et al. Genome-wide meta-analysis identifies new loci and functional pathways influencing Alzheimer's disease risk. Nat Genet 51, 404-413, doi:10.1038/s41588-018-0311-9 (2019).
23. International Multiple Sclerosis Genetics, C. Multiple sclerosis genomic map implicates peripheral immune cells and microglia in susceptibility. Science 365, doi:10.1126/science.aav7188 (2019).
24. Ozdinler, P. H. et al. Corticospinal motor neurons and related subcerebral projection neurons undergo early and specific neurodegeneration in hSOD1G(9)(3)A transgenic ALS mice. J Neurosci 31, 4166-4177, doi:10.1523/JNEUROSCI.4184-10.2011 (2011).
25. Nana, A. L. et al. Neurons selectively targeted in frontotemporal dementia reveal early stage TDP-43 pathobiology. Acta Neuropathol 137, 27-46, doi:10.1007/s00401-018-1942-8 (2019).
26. Porta, S. et al. Patient-derived frontotemporal lobar degeneration brain extracts induce formation and spreading of TDP-43 pathology in vivo. Nat Commun 9, 4220, doi:10.1038/s41467-018-06548-9 (2018).
27. Klim, J. R. et al. ALS-implicated protein TDP-43 sustains levels of STMN2, a mediator of motor neuron growth and repair. Nat Neurosci 22, 167-179, doi:10.1038/s41593-018-0300-4 (2019).
28. Tomassy, G. S. et al. Distinct profiles of myelin distribution along single axons of pyramidal neurons in the neocortex. Science 344, 319-324, doi:10.1126/science.1249766 (2014).
29. Giera, S. et al. The adhesion G protein-coupled receptor GPR56 is a cell-autonomous regulator of oligodendrocyte development. Nat Commun 6, 6121, doi:10.1038/ncomms7121 (2015).
30. Yang, H. J., Vainshtein, A., Maik-Rachline, G. & Peles, E. G protein-coupled receptor 37 is a negative regulator of oligodendrocyte differentiation and myelination. Nat Commun 7, 10884, doi:10.1038/ncomms10884 (2016).
31. Keren-Shaul, H. et al. A Unique Microglia Type Associated with Restricting Development of Alzheimer's Disease. Cell 169, 1276-1290 e1217, doi:10.1016/j.cell.2017.05.018 (2017).
32. de Boer, A. S. et al. Genetic validation of a therapeutic target in a mouse model of ALS. Sci Transl Med 6, 248ra104, doi:10.1126/scitranslmed.3009351 (2014).
33. Zhang, Y. et al. The C9orf72-interacting protein Smcr8 is a negative regulator of autoimmunity and lysosomal exocytosis. Genes Dev 32, 929-943, doi:10.1101/gad.313932.118 (2018).
34. Burberry, A. et al. C9orf72 suppresses systemic and neural inflammation induced by gut bacteria. Nature 582, 89-94, doi:10.1038/s41586-020-2288-7 (2020).
35. Subramanian, A. et al. A Next Generation Connectivity Map: L1000 Platform and the First 1,000,000 Profiles. Cell 171, 1437-1452 e1417, doi:10.1016/j.cell.2017.10.049 (2017).
36. Abud, E. M. et al. iPSC-Derived Human Microglia-like Cells to Study Neurological Diseases. Neuron 94, 278-293 e279, doi:10.1016/j.neuron.2017.03.042 (2017).
37. Nehme, R. et al. Combining NGN2 Programming with Developmental Patterning Generates Human Excitatory Neurons with NMDAR-Mediated Synaptic Transmission. Cell Rep 23, 2509-2523, doi:10.1016/j.celrep.2018.04.066 (2018).
38. Masuda, T. et al. Spatial and temporal heterogeneity of mouse and human microglia at single-cell resolution. Nature 566, 388-392, doi:10.1038/s41586-019-0924-x (2019).
39. Santillo, A. F. & Englund, E. Greater loss of von Economo neurons than loss of layer II and III neurons in behavioral variant frontotemporal dementia. Am J Neurodegener Dis 3, 64-71 (2014).
40. Genc, B. et al. Apical dendrite degeneration, a novel cellular pathology for Betz cells in ALS. Sci Rep 7, 41765, doi:10.1038/srep41765 (2017).
41. Wainger, B. J. et al. Effect of Ezogabine on Cortical and Spinal Motor Neuron Excitability in Amyotrophic Lateral Sclerosis: A Randomized Clinical Trial. JAMA Neurol 78, 186-196, doi:10.1001/jamaneurol.2020.4300 (2021).
42. O'Rourke, J. G. et al. C9orf72 is required for proper macrophage and microglial function in mice. Science 351, 1324-1329, doi:10.1126/science.aaf1064 (2016).
43. McCauley, M. E. et al. C9orf72 in myeloid cells suppresses STING-induced inflammation. Nature 585, 96-101, doi:10.1038/s41586-020-2625-x (2020).
44. Krienen, F. M. et al. Innovations present in the primate interneuron repertoire. Nature 586, 262-269, doi:10.1038/s41586-020-2781-z (2020).
45. Saunders, A. et al. Molecular Diversity and Specializations among the Cells of the Adult Mouse Brain. Cell 174, 1015-1030 e1016, doi:10.1016/j.cell.2018.07.028 (2018).
46. Raudvere, U. et al. g:Profiler: a web server for functional enrichment analysis and conversions of gene lists (2019 update). Nucleic Acids Res 47, W191-W198, doi:10.1093/nar/gkz369 (2019).
47. Koopmans, F. et al. SynGO: An Evidence-Based, Expert-Curated Knowledge Base for the Synapse. Neuron 103, 217-234 e214, doi:10.1016/j.neuron.2019.05.002 (2019).
48. Szklarczyk, D. et al. STRING vii: protein-protein association networks with increased coverage, supporting functional discovery in genome-wide experimental datasets. Nucleic Acids Res 47, D607-D613, doi:10.1093/nar/gky1131 (2019).
49. Subramanian, A. et al. Gene set enrichment analysis: a knowledge-based approach for interpreting genome-wide expression profiles. Proc Natl Acad Sci USA 102, 15545-15550, doi:10.1073/pnas.0506580102 (2005).

What is claimed is:

1. A method of treating a neurodegenerative disease or disorder comprising administering to a subject an agent, wherein the agent modulates neuronal regeneration.

2. The method of claim 1, wherein the agent modulates uptake of toxic proteins from intercellular environment.

3. The method of claim 1, wherein the agent increases uptake of toxic proteins from intercellular environment.

4. The method of claim 1, wherein the agent increases expression of SORL1.

5. The method of claim 1, wherein the agent increases expression of SORL1 in microglia and/or neurons.

6. The method of claim 1, wherein the neurodegenerative disease or disorder is amyotrophic lateral sclerosis.

7. A method of treating a neurodegenerative disease or disorder comprising administering to a subject an agent, wherein the agent modulates proteasome inhibition toxicity.

8. The method of claim 7, wherein the agent protects neurons from proteasome inhibition.

9. The method of claim 7, wherein the agent decreases expression of PSMD12.

10. The method of claim 7, wherein the agent decreases expression of PSMD12 in neurons.

11. The method of claim 7, wherein the neurodegenerative disease or disorder is amyotrophic lateral sclerosis.

12. A pharmaceutical composition comprising an agent and a pharmaceutically acceptable carrier, diluent, or excipient,
wherein the agent increases expression of SORL1 in microglia and/or neurons, or
wherein the agent decreases expression of PSMD12 in neurons.

13. The pharmaceutical composition of claim 12, wherein the agent increases expression of SORL1 in microglia and/or neurons.

14. The pharmaceutical composition of claim 13, wherein the composition modulates uptake of toxic proteins from an intercellular environment.

15. The pharmaceutical composition of claim 12, wherein the agent decreases expression of PSMD12 in neurons.

16. The pharmaceutical composition of claim 15, wherein the composition protects neurons from proteasome inhibition.

17. The pharmaceutical composition of claim 12, further comprising an agent for treating a neurodegenerative disease or disorder.

18. A method of screening one or more test agents to identify candidate agents for treating a neurodegenerative disease or condition in a subject, comprising
providing a neuronal cell having decreased expression of SORL1;
contacting the cell with one or more test agents;
determining if the contacted cell has an increased expression level of SORL1; and
identifying the test agent as a candidate agent if the contacted cell has an increased expression level of SORL1.

19. The method of claim 17, wherein the expression of SORL1 is measured using an ELISA assay.

20. The method of claim 17, wherein the neurodegenerative disease or condition is amyotrophic lateral sclerosis.
